(19) United States
(12) Patent Application Publication
(10) Pub. No.: US 2026/0008784 A1
(43) Pub. Date: Jan. 8, 2026

(54) COMPOUNDS, PHARMACEUTICAL COMPOSITIONS CONTAINING THEM AND THEIR MEDICAL USE FOR THE TREATMENT OR PREVENTION OF VASCULAR DISEASES

(71) Applicant: VASTHERA CO., LTD., Seoul (KR)

(72) Inventors: Sang Won KANG, Seoul (KR); Kyung Joo LEE, Seoul (KR); Dong Hoon KANG, Goyang-si (KR); Hiteshkumar Bhanubhai JALANI, Ahmedabad (IN); Doo Jae LEE, Seoul (KR); Sung Hoon JUNG, Seoul (KR)

(21) Appl. No.: 18/880,232

(22) PCT Filed: Jun. 28, 2023
(86) PCT No.: PCT/KR2023/009076

§ 371 Date: Dec. 30, 2024

(30) Foreign Application Priority Data
Jun. 29, 2022 (KR) 10-2022-0080028

(51) Int. Cl. C07D 513/08; A61K 31/554; A61P 9/12; C07D 513/18

(52) U.S. Cl. CPC C07D 513/08; A61K 31/554; A61P 9/12; C07D 513/18

(57) ABSTRACT

The present disclosure provides compounds capable of exhibiting effects similar to those of 2-Cys-peroxyredoxin (2-Cys-Prx) in the body with excellent pharmacological effects and reduced side effects such as reduced cytotoxicity, and pharmaceutical uses thereof. The compounds of the present disclosure and their pharmaceutically acceptable salts are useful for the treatment or prevention of vascular diseases, particularly ischemic coronary artery disease, arteriosclerosis, vascular restenosis, or pulmonary arterial hypertension. The compounds of the present invention and their pharmaceutically acceptable salts are particularly useful for the treatment or prevention of pulmonary arterial hypertension. The invention also provides methods of preparing the compounds of the present disclosure.

### TECHNICAL FIELD

The present disclosure relates to compounds exhibiting 2-Cys-peroxiredoxin (2-Cys-Prx) peroxidase mimetic activity and pharmaceutically acceptable salts thereof. The present disclosure also relates to pharmaceutical compositions for preventing or treating vascular diseases. That is, the present disclosure also relates to medical uses of the compounds disclosed herein and pharmaceutically acceptable salts thereof. The present disclosure also relates to preparing methods of compounds exhibiting 2-Cys-Prx activity mimetic effects and pharmaceutically acceptable salts thereof.

### BACKGROUND ART

Arterial vascular diseases refer to the state in which fatty substances (plaques) containing cholesterol, phospholipid, calcium, etc. accumulate in the intima of blood vessels, thereby resulting in inflammation, loss of elasticity and narrowing of arteries, resulting in impaired blood supply or increased pressure, and finally resulting in rupture or detachment of blood vessels. In particular, the blockage or occlusion of arterial vessels reduces blood supply and results in a lack of nutrients and oxygen, which is a major cause of vascular diseases. The vascular diseases generally include cardiovascular diseases such as arteriosclerosis, heart failure, hypertensive heart disease, arrhythmias, myocardial infarction, and angina pectoris, and cerebrovascular diseases such as stroke and peripheral vascular diseases.

Methods for overcoming such vascular occlusion include arterial transplantation by a surgical method, and percutaneous angioplasty, which is a method of expanding blood vessels using a balloon stent. Restenosis refers to a case in which the stenosis of the vessel diameter is greater than 50% on follow-up angiography after angioplasty. Although the incidence of restenosis has decreased as the stent materials are improved, restenosis still occurs at a rate of about 30% in patients who have undergone angioplasty (balloon dilatation and stent insertion). Although the mechanism of restenosis has not yet been precisely identified, it is known that growth factors and cytokines are locally secreted due to the damage of vascular endothelial cells during the procedure, and induce proliferation and migration of vascular smooth muscle, which thus narrows the arterial lumen and leads to restenosis. Therefore, hyperplasia of smooth muscle cells has recently been raised as a major clinical problem that limits the efficiency of angioplasty. Therefore, an improved stent that releases a drug inhibiting the proliferation of vascular smooth muscle cells was developed and used clinically. However, drugs currently used for this purpose prevent hyperplasia of the intimal layer through a cytotoxic mechanism of killing vascular smooth muscle cells, and thus has a limitation in that the toxicity leads to the death of not only smooth muscle cells but also endothelial cells. Hence, there is an urgent need to develop a drug with therapeutic potential capable of inhibiting the growth of vascular smooth muscle cells while se-lectively promoting recovery of the damaged endothelial cell layer.

On the other hand, the pulmonary artery is a blood vessel that carries blood from the right ventricle of the heart to the lungs to refresh with oxygen. Pulmonary arterial hypertension is defined as a mean pulmonary artery pressure at rest of 25 mmHg or higher and a mean pulmonary artery pressure of 30 mmHg during exercise. Pulmonary arterial hypertension is divided into “idiopathic pulmonary arterial hypertension”, in which no specific cause has been identified, heritable, and “associated pulmonary arterial hypertension”, which is associated with other diseases. The latter is associated with collagen vascular diseases (systemic sclerosis, lupus erythematosus, etc.), portal hypertension, HIV infection, congenital heart disease, or diseases caused by drugs or toxins such as appetite suppressants or cocaine. Pulmonary arterial hypertension associated with these specific diseases is not significantly different from idiopathic pulmonary arterial hypertension in terms of natural progression, histopathological findings, and response to treatment.

Currently, there is a method using a general vasodilator for the treatment of pulmonary arterial hypertension. Most vasodilators are calcium channel blockers. However, calcium channel blockers often do not show a significant effect on pulmonary arterial hypertension, and there are many side effects. Therefore, there is a need to discover effective therapeutic agents for pulmonary arterial hypertension.

On the other hand, “PrxII (also referred to as peroxiredoxin II or Prx2)” is one of the 2-Cys-Prx peroxidases that reduce intracellular hydrogen peroxide (H₂O₂). PrxII eliminates hydrogen peroxide produced by platelet-derived growth factor (PDGF) in vascular smooth muscle cells, inhibits site-specific phosphorylation of PDGFRβ and PCL (phospholipase C)γ1, and thereby inhibits signaling amplification. Through this mechanism, PrxII inhibits the proliferation and migration of smooth muscle cells and reverses the intimal thickening in damaged blood vessels (M. H. Choi et al., Nature 2005 May 19; 435(7040):347-53). However, it has been reported that PrxII protects VEGFR2 from oxidative inactivation and activates VEGF-induced signaling in vascular endothelial cells (D. H. Kang et al., Mol Cell. 2011 Nov. 18; 44(4):545-58).

PCT Publication WO2013-077709 confirmed that natural compounds having an epidithiodioxopiperazine structure can exhibit the 2-Cys-Prx-like activity in cells. By mimicking the 2-Cys-Prx activity, the natural compounds inhibit PDGF-induced proliferation and migration in vascular smooth muscle cells and promote VEGF-induced proliferation and migration in vascular endothelial cells. Furthermore, it demonstrated that the natural compounds inhibit intimal thickening caused by excessive proliferation of vascular smooth muscle cells and promote recovery of vascular endothelial layer in an experimental animal model and ultimately it implicates such compounds be useful for preventing or treating vascular diseases.

In addition, PCT Publication WO2018-008984 confirmed that a drug capable of mimicking the intracellular activity of 2-Cys-Prx peroxidase may be useful for the treatment or prevention of pulmonary arterial hypertension.

However, in the case of the compounds disclosed in WO2013-077709 and WO2018-008984, the aforementioned toxicity problem has not been completely resolved. Therefore, there is a steady need for an active agent that exhibits the advanced pharmacological effect and improved adverse cytotoxicity.

### DISCLOSURE OF INVENTION

### Technical Problem

The goal of the present disclosure is to provide compounds that exhibit similar effects to 2-Cys-peroxiredoxin (2-Cys-Prx) in the body and have excellent pharmacological effects and reduced side effects such as reduced cytotoxicity, and medicinal uses thereof.

Another goal to be solved by the present disclosure is to provide pharmaceutical composition(s) for treating or preventing vascular disease(s), which comprises as an active ingredient a compound having excellent pharmacological effects and reduced side effects such as reduced toxicity. That is, the goal to be solved by the present disclosure is to provide a method for treating or preventing vascular disease(s), comprising administering a therapeutically effective amount of the compound or salt thereof of the present disclosure to a subject in need of treatment or prevention of vascular disease(s).

The other problem to be solved by the present invention is to provide a method for preparing the specific compound according to the present disclosure.

### Solution to Problem

In order to solve the above problem, one embodiment of the present invention provides a compound represented by the following Chemical Formula 1 or 2, or a pharmaceutically acceptable salt thereof.

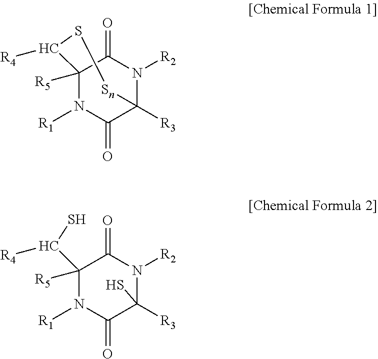

In the Chemical Formula 1 and 2,
n is an integer of from 1 to 3,
R₁ and R₂ are each independently C₁₋₃alkyl (preferably methyl or ethyl), C₁₋₃alkoxy-C₁₋₃alkyl, —(CH₂)₁₋₃—C(R′)(R″)OH, —(CH₂)₁₋₃—N(R′)(R″), —(CH₂)O₃-alkenyl, —(CH₂)₀₋₃-alkynyl, —(CH₂)₀₋₃—C(R′)(R″)CO₂H, —(CH₂)₀₋₅-heterocycloalkyl, —(CH₂)₀₋₅-cycloalkyl, —(CH₂)₀₋₅-aryl (preferably —CH₂-phenyl), or —(CH₂)₀₋₅-heteroaryl (preferably —CH₂— pyridyl, —CH₂-quinolinyl, —CH₂-pyrazolyl, —CH₂-thiophen-2-yl, —CH₂-benzo[d]thiazol-2-yl, —CH₂-pyrimidyl, or —CH₂-1H-imidazol-4-yl), wherein the alkyl, heterocycloalkyl, cycloalkyl, aryl and heteroaryl are unsubstituted or optionally substituted with one or more substituents selected from the group consisting of C₁₋₃alkyl, —CF₃, C₁₋₃alkoxy, —OCF₃, halogen (preferably F), CN, amino, —N(R′)(R″), —OH, —COOH, —COO—C₁₋₃alkyl, and =O, wherein R′ and R″ are each independently H or C₁₋₃alkyl,
R₃ is C₁₋₃alkyl (preferably methyl), —(CH₂)₀₋₃-aryl, or —(CH₂)₀₋₃-heteroaryl, wherein the aryl or heteroaryl is unsubstituted or optionally substituted with one or more substituents selected from the group consisting of C₁₋₃alkyl, —CF₃, C₁₋₃alkoxy, —OCF₃, halogen, —CN, amino, —OH, and —COOH; or
R₂ and R₃ are linked together and fused with piperazinedione present in Chemical Formula 1 to form one of the following structures:

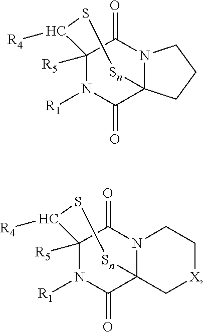

wherein,
X is S, SO₂, CH₂, O or NR₆, wherein R₆ is H or C₁₋₃alkyl,
R₄ is H or C₁₋₃alkyl, and
R₅ is H, C₁₋₃alkyl, —(CH₂)₁₋₂-aryl, or —(CH₂)₁₋₂-heteroaryl.

Another embodiment of the present invention provides a compound represented by the above Chemical Formula 1 or 2, or a pharmaceutically acceptable salt thereof, wherein
n is an integer of from 1 to 3,
R₁ and R₂ are each independently C₁₋₃alkyl, —(CH₂)₁₋₂-heterocycloalkyl, —(CH₂)₁₋₂-aryl, or —(CH₂)₁₋₂-heteroaryl, wherein the alkyl, heterocycloalkyl, aryl, and heteroaryl are unsubstituted or optionally substituted with one or more substituents selected from the group consisting of C₁₋₃alkyl, —CF₃, C₁₋₃alkoxy, CN, halogen, —OH, —COOH, and —COO—C₁₋₃alkyl,
R₃ is C₁₋₃alkyl, —CH₂-aryl, or —CH₂-heteroaryl, wherein the aryl or heteroaryl is unsubstituted or optionally substituted with one or more substituents selected from the group consisting of methyl, methoxy, halogen, —CN, amino, —OH, and —COOH; or
R₂ and R₃ are linked together and fused with piperazinedione present in Chemical Formula 1 to form one of the following structures:

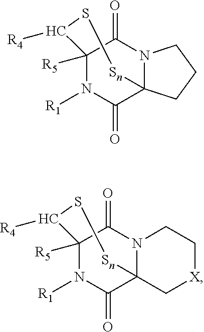

wherein,
X is S, SO₂, CH₂, O or NR₆, wherein R₆ is H or C₁₋₃alkyl,
R₄ is H or C₁₋₃alkyl, and
R₅ is H, C₁₋₃alkyl, or —(CH₂)₁₋₂-aryl.

Bridged Epidithiodioxopiperazine-based compounds according to the present invention are a new structure that does not exist in natural products unlike ETP (EpidiThiodioxoPiperazine) derivatives which are often found in natural products. As a result of confirmation by the present inventors, the compounds of the present invention having such a structure have very high chemical and biological stability, and have better pharmacological activity and reduced toxicity compared to the existing ETP derivative compounds disclosed in WO2013-077709.

In the compounds of the present invention, one methylene is additionally introduced (i.e., bridged ETP) compared to the existing ETP derivatives disclosed in WO2013-077709, thereby imparting considerable stability to the disulfide structure. In other words, when comparing the dihedral angle of disulfide bonds, existing ETP derivatives have a high reactivity due to considerable stress on the ring of around 10°, whereas the compounds of the present invention have around 50-60° as shown in the results of X-ray analysis, which means that the stress on the ring is relatively low. Relief of this ring stress is expected to play a role in lowering toxicity by lowering the indiscriminate reactivity of the compound, and at the same time, it is expected to increase redox reactivity with thioredoxin (Trx) protein due to increased flexibility. However, the present invention is not limited to these theoretical mechanisms.

In addition, the compounds of the present invention have structural features that fa-cilitate interaction with the thiol group of the cysteine residue of the thioredoxin C-X-X-C motif, which is an electron donor for hydrogen peroxide reduction, and thereby are expected to exhibit more specific and high activity. For example, in the case of conventional ETP derivatives disclosed in WO2013-077709, pharmacological activity is poor with bulky substituents, but in the compounds of the present invention, it shows good activity even with large structural substituents such as aryl and heteroaryl. However, the present invention is not limited to such theoretical speculation.

In the case of a trisulfide compound having three sulfurs or a tetrasulfide compound having four sulfurs in the present disclosure, the connection between sulfurs in the body may be released and then connected to a disulfide compound. In this case, trisulfide compounds with three sulfurs or tetrasulfide compounds with four sulfurs are expected to act as prodrugs for disulfide compounds with two sulfurs. However, the present invention is not limited to such theoretical speculation.

If a substituent is described as “optionally substituted”, the substituent may be (1) unsubstituted or (2) substituted with one or more of the defined substituents. If the sub-stitutable position is unsubstituted, the default substituent is hydrogen.

As used herein, the term “alkyl” means a saturated straight chain or branched non-cyclic hydrocarbon, unless the context clearly dictates otherwise, having from 1 to 10 carbon atoms. “Lower alkyl” means alkyl having from 1 to 4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl, while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. In a preferred embodiment of the present invention, alkyl is methyl.

As used herein, the term “alkenyl” means a saturated straight or branched non-cyclic hydrocarbon containing 2 to 10 carbon atoms and at least one carbon-carbon double bond. Representative straight-chain and branched (C₂-C₁₀) alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-disenyl, -2-disenyl, and -3-disenyl. In one embodiment of the present invention, alkenyl is vinyl or allyl.

As used herein, the term “alkynyl” means a straight chain or branched non-cyclic hydrocarbon, unless the context clearly dictates otherwise, having 2 to 10 carbon atoms and containing at least one carbon-carbon triple bond. Representative linear or branched (C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-noninyl, -2-noninyl, -8-noninyl, -1-decynyl, -2-decynyl, and -9-decynyl. In one embodiment of the present invention, alkynyl is -ethynyl or -propynyl.

As used herein, if the term “C₁₋₆”, “C1-6”, “C₁-C₆”, or “C1-C6” is used, it means the number of carbon atoms is from 1 to 6. For example, C₁₋₆alkyl means an alkyl which carbon number is any integer of from 1 to 6.

As used herein, the term “C₁₋₆alkoxy” means —O-(alkyl), where alkyl is as defined above. For example, methoxy, ethoxy, n-propoxy, 1-methylethoxy (isopropoxy), butoxy, 1-methylpropoxy (sec-butoxy), 2-methylpropoxy (isobutoxy), 1,1-dimethylethoxy (tert-butoxy), pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy may be exemplified.

As used herein, the terms “halogen” and “halo” mean fluorine, chlorine, bromine or iodine.

As used herein, the term “cycloalkyl” means a monocyclic or polycyclic saturated ring having carbon and hydrogen atoms and having no carbon-carbon multiple bonds, and is C₃₋₇cycloalkyl having 3 to 7 carbon atoms unless the context clearly dictates otherwise. Examples of monocyclic rings include, but are not limited to, (C₃-C₇)cycloalkyl groups, including cyclopropyl, cyclobutyl, cyclopentyl, cyclohexyl, and cycloheptyl. Examples of polycyclic rings include, but are not limited to, fused bicyclic rings such as octahydropentalene and decahydronaphthalene; spiro rings such as spiro[3.3]heptane, spiro[3.4]octane, spiro[3.5]nonane, spiro[4.4]nonane, spiro[4.5]decane, and spiro[5.5]undecane; and bridged bicycle rings such as bicyclo[2.1.1]hexane, bicyclo[2.2.1]heptane, and bicyclo[2.2.2]octane. A cycloalkyl group can be unsubstituted or optionally substituted. In an embodiment of the present invention, cycloalkyl is monocyclic ring.

The term “heterocycle” or “heterocycloalkyl” means a 4- to 7-membered monocyclic, or 7- to 12-membered bicyclic saturated ring which contains from 1 to 4 heteroatoms independently selected from nitrogen, oxygen and sulfur, and wherein the nitrogen and sulfur heteroatoms can be optionally oxidized, and the nitrogen heteroatom can be optionally quaternized. “Heterocycloalkyl” means “hetero(C₃₋₇)cycloalkyl” unless the context clearly dictates otherwise. Representative heterocycles include oxirane, oxetane, tetrahydrofuran, tetrahydropyran, 1,4-dioxane, aziridine, azetidine, pyrrolidine, piperidine, piperazine, pyrrolidinone, hydantoine, valerolactam, thiirane, thietane, tetrahydrothiophene, tetrahydrothiopyran, morpholine, tetrahydropyridine, tetrahydropyrimidine, and the like. Heterocycles include a bicyclic ring in which part of the heterocycle is fused to a benzene or cyclopenta-1,3-diene ring. The heterocycle can be attached via any heteroatom or carbon atom. In addition, heterocycles include fused bicyclic rings, spiro rings and bridged bicyclic rings in which one or more carbon atoms of the aforementioned polycyclic rings are replaced with nitrogen, oxygen or sulfur atoms. For example, when the heteroatom is nitrogen, these include, but are limited to, fused heterobicyclic rings such as octahydrocyclopenta[c]pyrrole, octahydropyrrolo[3,4-c]pyrrole, decahydroisoquinoline, and decahydro-2,6-naphthyridine; spiro rings such as 2-azaspiro[3.3]heptane, 2,6-diazaspiro[3.3]heptane, 2-azaspiro[3.4]octane, 2,6-diazaspiro[3.4]octane, 2-azaspiro[3.5]nonane, 2,7-diazaspiro[3.5]nonane, 2-azaspiro[4.4]nonane, 2,7-diazaspiro[4.4]nonane, 8-azaspiro[4.5]decane, 2,8-diazaspiro[4.5]decane, 3-azaspiro[5.5]undecane, and 3,9-diazaspiro[5.5]undecane; and bridged heterobicyclic rings such as 2-azabicyclo[2.1.1]hexane, 2-azabicyclo[2.2.1]heptane, 2,5-diazabicyclo[2.2.1]heptane, 2-azabicyclo[2.2.2]octane, and 2,5-diazabicyclo[2.2.2]octane. In an embodiment of the present invention, heterocycle is piperidyl, morpholinyl, or piperazinyl.

As used herein, the term “aryl” means a carbocyclic aromatic group containing from 5 to 10 ring atoms. Representative examples include, but are not limited to, phenyl, tolyl, xylyl, naphthyl, tetrahydronaphthyl, anthracenyl, fluorenyl, indenyl, and azulenyl. A carbocyclic aromatic group can be unsubstituted or optionally substituted. In an embodiment of the present invention, aryl is phenyl or naphthyl.

As used herein, the term “heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls include furan, 4H-pyran, pyrrole, imidazole, pyrazole, triazole, tetrazole, pyridine, pyrimidine, pyridazine, pyrazine, triazine, thiophene, ozaxole, isoxazole, thiazole, isothiazole, oxadiazole, benzofuran, ben-zothiophene, quinoline, indole, benzoxazole, benzimidazole, benzothiazole, cinnoline, phthalazine, quinazoline, 1H-azepine, etc. In an embodiment of the present invention, heteroaryl is pyridine, quinoline, pyrazole, thiophene, benzo[d]thiazole, pyrimidine, imidazole, thiazole, isoxazole, indole, quinazoline, or benzimidazole.

A preferred embodiment of the present invention provides a compound represented by the above Chemical Formula 1 or 2, or a pharmaceutically acceptable salt thereof, wherein
n is 1,
R₁ and R₂ are each independently C₁₋₃alkyl, —CH₂-piperidyl, —CH₂-morpholinyl, —CH₂— piperazinyl, —CH₂-phenyl, —CH₂-naphthyl, —CH₂-pyridyl, —CH₂-quinolinyl, —CH₂— pyrazolyl, —CH₂-thiophen-2-yl, —CH₂-benzo[d]thiazol-2-yl, —CH₂-pyrimidyl, —CH₂-1H-imidazol-4-yl, —CH₂-1H-imidazol-2-yl, —CH₂-thiazol-4-yl, —CH₂-thiazol-5-yl, —CH₂— isoxazolyl, —CH₂-indol-2-yl, —CH₂-indol-3-yl, —CH₂-benzimidazol-5-yl, —CH₂-quinolin-4-yl, —CH₂-quinazol-2-yl, or —CH₂-quinazol-4-yl, wherein the the piperidyl, morpholinyl, piperazinyl, phenyl, naphthyl, pyridyl, quinolinyl, pyrazolyl, thiophene, benzo[d]thiazole, pyrimidyl, imidazole, thiazole, isoxazolyl, indole, benzimidazole, quinoline and quinazole are unsubstituted or optionally substituted with one or more substituents selected from the group consisting of C₁₋₃alkyl, —CF₃, C₁₋₃alkoxy, CN, halogen, and —COO—C₁₋₃alkyl,
R₃ is C₁₋₃alkyl or —CH,-aryl, or
R₂ and R₃ are linked together and fused with piperazinedione present in Chemical Formula 1 to form one of the following structures:

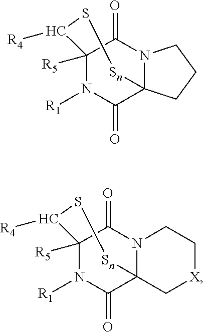

wherein,
X is O or NR₆, wherein R₆ is methyl,
R₄ is H, and
R₅ is H.

Among the compounds of the Chemical Formula 1,
1,6,8-trimethyl-2,3-dithia-6,8-diazabicyclo[3.2.2]nonan-7,9-dione (Compound 1),
6-benzyl-1,8-dimethyl-2,3-dithia-6,8-diazabicyclo[3.2.2]nonan-7,9-dione (Compound 2),
1,8-dimethyl-6-(3,4,5-trimethoxybenzyl)-2,3-dithia-6,8-diazabicyclo[3.2.2]nonan-7,9-dione (Compound 3),
6-(3,5-difluorobenzyl)-1,8-dimethyl-2,3-dithia-6,8-diazabicyclo[3.2.2]nonan-7,9-dione (Compound 4),
1,8-dimethyl-6-(quinolin-2-ylmethyl)-2,3-dithia-6,8-diazabicyclo[3.2.2]nonan-7,9-dion e (Compound 5),
1,8-dimethyl-6-(pyridin-2-ylmethyl)-2,3-dithia-6,8-diazabicyclo[3.2.2]nonan-7,9-dione (Compound 6),
11-benzyltetrahydro-5H,7H-4,9a-(epiminomethano)pyrrolo[2,1-c][1,2,4]dithiazepin-5, 10-dione (Compound 7),
12-benzyltetrahydro-5H,10H-4,10a-(epiminomethano)[1,4]oxazino[3,4-c][1,2,4]dithia zepin-5,11-dione (Compound 8),
12-(pyridin-4-ylmethyl)tetrahydro-5H,10H-4,10a-(epiminomethano)[1,4]oxazino[3,4-c][1,2,4]dithiazepin-5,11-dione (Compound 9),
12-ethyltetrahydro-5H,10H-4,10a-(epiminomethano)[1,4]oxazino[3,4-c][1,2,4]dithiaze pin-5,11-dione (Compound 10),
11-(pyridin-4-ylmethyl)tetrahydro-5H,7H-4,9a-(epiminomethano)pyrrolo[2,1-c][1,2,4]dithiazepin-5,10-dione (Compound 11),
1,8-dimethyl-6-((6-methylpyridin-2-yl)methyl)-2,3-dithia-6,8-diazabicyclo[3.2.2]nona n-7,9-dione (Compound 12),
1,8-dimethyl-6-((1-methyl-1H-pyrazol-4-yl)methyl)-2,3-dithia-6,8-diazabicyclo[3.2.2]nonan-7,9-dione (Compound 13),
1,8-dimethyl-6-(thiophen-2-ylmethyl)-2,3-dithia-6,8-diazabicyclo[3.2.2]nonan-7,9-dio ne (Compound 14),
6-(benzo[d]thiazol-2-ylmethyl)-1,8-dimethyl-2,3-dithia-6,8-diazabicyclo[3.2.2]nonan-7,9-dione (Compound 15),
1,6-dimethyl-8-(pyrimidin-2-ylmethyl)-2,3-dithia-6,8-diazabicyclo[3.2.2]nonan-7,9-di one (Compound 16),
1,6-dimethyl-8-((1-methyl-1H-imidazol-4-yl)methyl)-2,3-dithia-6,8-diazabicyclo[3.2.2]nonan-7,9-dione (Compound 17), methyl
4-((1,6-dimethyl-7,9-dioxo-2,3-dithia-6,8-diazabicyclo[3.2.2]nonan-8-yl)methyl)benzo ate (Compound 18),
1-benzyl-6,8-dimethyl-2,3-dithia-6,8-diazabicyclo[3.2.2]nonan-7,9-dione (Compound 19), 6,8-diethyl-1-methyl-2,3-dithia-6,8-diazabicyclo[3.2.2]nonan-7,9-dione (Compound 20),
12-benzyl-9-methylhexahydro-5H-4,10a-(epiminomethano)pyrazino[2,1-c][1,2,4]dithi azepin-5,11-dione (Compound 21),
12-benzyl-9-methylhexahydro-5H-4,10a-(epiminomethano)pyrazino[2,1-c][1,2,4]dithi azepin-5,11-dione (Compound 22),
12-benzyl-9-methylhexahydro-5H-4,10a-(epiminomethano)pyrazino[2,1-c][1,2,4]dithi azepin-5,11-dione (Compound 23),
6-benzyl-1,4,8-trimethyl-2,3-dithia-6,8-diazabicyclo[3.2.2]nonan-7,9-dione (Compound 24), and
6-benzyl-1,4,8-trimethyl-2,3-dithia-6,8-diazabicyclo[3.2.2]nonan-7,9-dione (Compound 25) are particularly preferable in terms of various aspects such as 2-Cys-Prx mimetic activity and safety.

The compound of Chemical Formula 1 or 2 of the present invention may be used in the form of a pharmaceutically acceptable salt.

As used herein, the term “pharmaceutically acceptable salt” refers to any salt that retains the desired biological and/or physiological activity of the compounds and exhibits minimal undesirable toxicological effects. In the present invention, any type of salt may be used without limitation as long as the diketopiperazine ring containing intramolecular disulfide bridge is maintained. As the salt, an acid addition salt formed by a pharmaceutically acceptable free acid is useful. Acid addition salts are prepared by conventional methods, for example, by dissolving the compound in an excess aqueous solution and precipitating the salt using a water-miscible organic solvent, such as methanol, ethanol, acetone or acetonitrile. Equimolar amounts of the compound and an acid or alcohol (e.g., glycol monomethyl ether) in water can be heated, and then the mixture can be evaporated to dryness, or the precipitated salt can be filtered by suction. At this time, inorganic acids and organic acids may be used as the free acid. Hy-drochloric acid, hydrobromic acid, phosphoric acid, nitric acid, sulfuric acid, tartaric acid, etc. may be used as the inorganic acid, and methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hy-droiodic acid, etc. may be used as the organic acid. However, the acid used in the present disclosure is not limited thereto.

In addition, a pharmaceutically acceptable metal salt may be prepared using a base. Alkali metal or alkaline earth metal salts are obtained, for example, by dissolving the compound in an excess alkali metal hydroxide or alkaline earth metal hydroxide solution, filtering the undissolved compound salt, and then evaporating and drying the filtrate. At this time, as the metal salt, it is particularly suitable for preparing sodium, potassium or calcium salts, but is not limited thereto. In addition, the corresponding silver salt can be obtained by reacting an alkali metal or alkaline earth metal salt with a suitable silver salt (e.g., silver nitrate).

Pharmaceutically acceptable salts of the compounds according to the present invention include all salts of acidic or basic groups which may be present, unless otherwise indicated. For example, pharmaceutically acceptable salts may include sodium, calcium, and potassium salts of a hydroxyl group, and other pharmaceutically acceptable salts of an amino group include hydrobromide, sulfate, hydrogen sulfate, phosphate, hydrogen phosphate, hydrogen phosphate, acetate, succinate, citrate, tartrate, lactate, mandelate, methanesulfonate (mesylate) and p-toluenesulfonate (tosylate) salts, etc. These can be manufactured through a salt preparation method known in the art.

As used herein, the phrase “compound(s) of this/the invention” includes any compound(s) of Chemical Formula 1, as well as clathrates, hydrates, solvates, or polymorphs thereof. And, even if the term “compound(s) of the invention” does not mention its pharmaceutically acceptable sat, the term includes salts thereof. In one embodiment, the compounds of this disclosure include stereo-chemically pure compounds, e.g., those substantially free (e.g., greater than 85% ee, greater than 90% ee, greater than 95% ee, greater than 97% ee, or greater than 99% ee) of other stereoisomers. That is, if the compounds of Chemical Formula 1 according to the present disclosure or salts thereof are tautomeric isomers and/or stereoisomers (e.g., geometrical isomers and conformational isomers), such isolated isomers and their mixtures also are included in the scope of this disclosure. If the compounds of the present disclosure or salts thereof have an asymmetric carbon in their structures, their active optical isomers and their racemic mixtures also are included in the scope of this disclosure.

As used herein, the term “polymorph” refers to solid crystalline forms of a compound of this disclosure or complex thereof. Different polymorphs of the same compound can exhibit different physical, chemical and/or spectroscopic properties. Different physical properties include, but are not limited to stability (e.g., to heat or light), compressibility and density (important in formulation and product production), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a ki-netically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.

As used herein, the term “purified” means that when isolated, the isolate is greater than 90% pure, in one embodiment greater than 95% pure, in another embodiment greater than 99% pure and in another embodiment greater than 99.9% pure.

“2-Cys-Prx” is a thiol-specific antioxidant enzyme that serves to protect cells through peroxidase activity that reduces hydrogen peroxide, peroxynitrite and other hy-droperoxides in cells. Its functional unit is a homodimer, which has a unique intramolecular redox active disulfide center that plays an important role in the activity of the enzyme. In the reduced state, each cysteine in the dimer exists as a thiol group. On the other hand, when reacting with a peroxide, the peroxidatic cysteine of 2-Cys-Prx is oxidized to a sulfenic acid intermediate, which is distinguished from the cysteine of other subunits of the dimer that retains the thiol group. The sulfenic acid group and thiol group of the intermediate undergo dehydration condensation to form intramolecular disulfide bridges. For example, in cells, 2-Cys-Trx is oxidized from a reduced form containing two thiol groups to an oxidized form in which the two thiol groups form the intramolecular disulfide bond, thereby reducing intracellular hy-droperoxides. At this time, the oxidized form of 2-Cys-Prx containing intramolecular disulfide bond can be converted to a reduced form, which is an active form with two thiol groups, through coupling with the redox system including thioredoxin (Trx) and thioredoxin reductase (Trx reductase; TR); AhpF (alkyl hydroperoxide reductase); trypanothione reductase, trypanothione and tryparedoxin or lipoamide dehydrogenase, di-hydrolipoyltranssuccinylase (SucB) and AhpD.

The compounds of the present invention mimic 2-Cys-Prx activity in cells as specifically coupled with thioredoxin (Trx) and thioredoxin reductase (Trx reductase; TR) system and exhibit excellent activity to inhibit the proliferation and migration of vascular smooth muscle cells induced by PDGF. In addition, the compounds of the present invention inversely promote proliferation or migration of vascular endothelial cells induced by VEGF.

Accordingly, one embodiment of the present invention provides a pharmaceutical composition for preventing or treating a vascular disease comprising the compound represented by Chemical Formula 1 or 2 or a pharmaceutically acceptable salt thereof as an active ingredient.

Another embodiment of the present invention provides a method of treating or preventing a vascular disease comprising administering a therapeutically effective amount of the compound represented by Chemical Formula 1 or 2 or a pharmaceutically acceptable salt thereof to a subject in need of prevention or treatment of the vascular disease or a subject suspected of the vascular disease.

The other embodiment of the present invention provides use of the compound represented by Chemical Formula 1 or 2 or a pharmaceutically acceptable salt thereof for the manufacture of a medicament for the treatment or prevention of a vascular disease.

In the pharmaceutical composition, method or use, the vascular disease may be hypertension (e.g., pulmonary arterial hypertension), ischemic coronary artery disease (e.g., angina pectoris, myocardial infarction, unstable angina), cerebral artery occlusion (e.g., stroke), atherosclerosis (e.g., coronary artery atherosclerosis, carotid artery atherosclerosis), peripheral arterial occlusive disease (e.g., Burgers' disease), thromboembolism, diabetic foot disease, venous ulcer, deep vein thrombosis, vasospasm, arteritis, and vascular smooth muscle hyperplasia, such as vascular restenosis and/or vascular diseases caused by angioendothelial loss. That is, the pharmaceutical composition of the present disclosure can be used for treating or preventing any of the above vascular diseases. In one embodiment of the present invention, the vascular disease is ischemic coronary artery disease, arteriosclerosis, vascular restenosis, or pulmonary arterial hypertension. In another embodiment of the present invention, the vascular disease is pulmonary arterial hypertension.

In one embodiment of the present invention, the vascular restenosis may be vascular restenosis caused by vascular transplantation, vascular dissection, arteriosclerosis, in-travascular fat accumulation, hypertension, vascular inflammation, or angioplasty. Although the reason for vascular restenosis has not been clarified, it is known that growth factors and/or cytokines are secreted from surrounding cells via mechanism for recovery after vascular damage due to various reasons or vascular endothelial damage caused by a device inserted during angioplasty procedure and these growth factors and/or cytokines cause abnormal migration and proliferation of vascular smooth muscle cells and thereby cause intimal thickening. The blood vessels include, but are not limited to, the aorta, the carotid artery, the coronary artery, the peripheral artery, the renal artery, and the like.

In one embodiment of the present invention, the pulmonary arterial hypertension is a type of hypertension affecting the arteries of the lungs and the right side of the heart, and refers to a case in which the mean pulmonary arterial pressure at rest is 25 mmHg or more and the mean pulmonary artery pressure during exercise is 30 mmHg or more. In one form of pulmonary arterial hypertension, the small arteries and capillaries in the lungs, called pulmonary arteries, may be narrowed, blocked, or damaged, which makes it difficult for blood to flow through the lungs and increasing pressure within the pulmonary arteries. As this puts pressure, it makes it difficult for the lower right chamber (right ventricle) to pump blood through the lungs and eventually causes right ventricle hypertrophy, which can lead to heart failure.

The composition according to the present invention may further include suitable carriers, excipients and diluents commonly used in the manufacture of pharmaceutical compositions. The composition is sterile or free of germs, and may be water, buffer, isotonic agent, etc., and the solution is sterile or no germs. The composition does not cause allergic or other harmful reactions when applied to animals or humans. The composition also may contain other ingredients known to the skilled person in the art.

As used herein, the term “pharmaceutically acceptable carrier” includes any and all solvents, dispersion media, coatings, antibacterial, antifungal and isotonic agents and the like. The use of such media and agents for pharmaceutically active carriers is well known in the art. In addition to the usual media or agents that are incompatible with the active ingredient, their use in therapeutic compositions is contemplated. Sup-plementary active ingredients may also be incorporated into the compositions.

The composition may be prepared in formulations such as liquid, emulsion, suspension or cream, and may be used parenterally. The amount of the composition can be used as a normal amount for the prevention or treatment of vascular diseases, and it is preferable to apply differently depending on the patient's age, sex, condition, absorption of active ingredients in the body, inactivation rate, and concomitant drugs, etc.

In the present invention, the term “prevention” refers to any action that suppresses or delays the onset of vascular disease by administration of the pharmaceutical composition according to the present invention, and the term “treatment” refers to any action that improve or beneficially change symptoms caused by vascular diseases by administration of the pharmaceutical composition.

In the present invention, the term “subject” refers to all animals, including humans, that have or may develop vascular disease, and the vascular disease can be effectively prevented or treated by administering the pharmaceutical composition of the present invention to the subject. In addition, the pharmaceutical composition of the present invention may be administered in combination with a known therapeutic agent for vascular diseases.

A compound of the present invention or a pharmaceutically acceptable salt thereof is administered in a therapeutically effective amount. The term “therapeutically effective amount” means an amount sufficient to treat a disease with a reasonable benefit/risk ratio applicable to medical treatment and not causing side effects, and the effective dose level is determined by the patient's sex, age, body weight, health status, severity of disease, activity of the drug, sensitivity to the drug, method of administration, time of administration, route of administration and excretion rate, duration of treatment, factors including drugs used in combination or concomitantly, and other factors well known in the medical field. It can be easily determined by a person skilled in the art.

For example, the compounds or pharmaceutically acceptable salts thereof according to the present invention can be administered by any suitable route in the form of a pharmaceutical composition adapted to such a route, and in a dose effective for the treatment intended. An effective dosage is typically in the range of about 0.0001 to about 100 mg per kg body weight per day, preferably about 0.001 to about 50 mg/kg/day, in single or divided doses. More preferably, the effective dosage is about 0.005 to about 20 mg/kg/day, in single or divided doses. Depending on age, species and disease or condition being treated, dosage levels below the lower limit of this range may be suitable. In other cases, still larger doses may be used without harmful side effects. Larger doses may also be divided into several smaller doses, for administration throughout the day.

As used herein, the term “administration” means introducing a predetermined substance into a patient by an appropriate method, and the administration route of the composition may be administered through any general route as long as it can reach the target tissue. As the administration method, oral administration, intravenous administration, subcutaneous administration, intraperitoneal administration, etc. may be used, but is not limited thereto. In one preferred embodiment of the invention, the compounds of the invention are administered orally. In another embodiment of the invention, a compound of the invention can be administered locally to a lesion. In particular, the compounds of the present invention can be administered using a drug eluting stent. That is, the compound of the present invention can be applied inside or on the stent and directly administered to the stenosis site. A double balloon catheter, a dispatcher, or a microporous balloon may be used for local drug administration, and in particular, a stent or sustained-release microparticles may be used to deliver the drug for a long period of time.

In addition, one embodiment of the present invention provides a drug delivery device for local administration comprising the pharmaceutical composition according to the present invention for preventing or treating vascular disease. The drug delivery device for local administration may include, but is not limited to, a double balloon catheter, a dispatcher, a microporous balloon, a stent, and the like, and may preferably be a stent.

As used herein, the term “stent” refers to a general device for endoluminal application, for example, application within a blood vessel. In particular, the stent refers to a cylindrical medical material inserted into a narrowed or clogged blood vessel under X-ray fluoroscopy to improve blood flow in the area where blood flow should be smooth but the flow is obstructed. In a preferred embodiment of the present invention, the stent is a sustained-release drug-releasing stent.

As a method of coating the pharmaceutical composition of the present invention on the stent, a conventional coating method known to those skilled in the art, for example, dip-coating and/or polymer coating method, may be applied. The dip-coating method is the simplest coating method, and since only the pharmaceutical composition is coated, it is easy to observe the biological effect of only the drug, but is not limited thereto. Preferably, the stent of the present invention can be prepared by mixing the composition according to the present invention with a polymer material and then coating the stent so that the composition can be slowly released. Polymeric materials that can be used for drug-eluting stents are well known in the art, and examples include polyurethane, polyethylene terephthalate, poly-lactic acid-poly-glycolic acid copolymer (PLGA), polycaprolactone, poly-(hydroxybutyrate/hydroxyvalerate) copolymer, polyvinylpyrrolidone, polytetrafluoroethylene, poly(2-hydroxyethyl methacrylate), poly(etherurethane urea), silicone, polyacrylic acid, polyepoxide, polyester, urethane, parylene, polyphosphazine polymer, fluoropolymer, polyamide, polyolefin, and mixtures thereof, but are not limited thereto.

The stent may be coated with at least one material selected from polysaccharides, heparin, gelatin, collagen, alginate, hyaluronic acid, alginic acid, carrageenan, chondroitin, pectin, chitosan, and derivatives and copolymers thereof, or may be further coated to form a layer comprising any one of them or an antithrombotic agent. Suitably, these materials may be incorporated into a biocompatible topcoat, as described in US 2006/0083772.

One embodiment of the present invention also provides a method of preparing a compound represented by Chemical Formula 1′, comprising a step of forming an intramolecular disulfide bridge from a 3-mercapto-6-methylmercapto piperazinedione compound represented by Chemical Formula 2 by oxidation reaction. As mentioned above, the compound represented by Chemical Formula 1′ has improved intracellular permeability and mimics 2-Cys-Prx activity by being reduced inside cells.

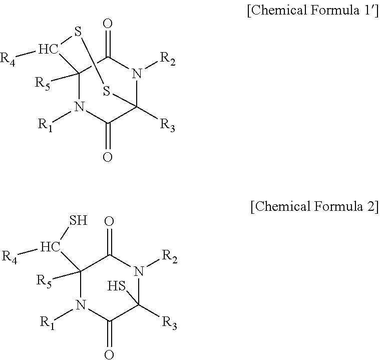

(In the Chemical Formula 1′ and 2, R₁, R₂, R₃, R₄ and R₅ are the same as in Chemical Formula 1 above.)

As mentioned above, unless otherwise stated, if stereoisomers are possible in the compounds represented by the Chemical Formulas of the present invention, these stereoisomers are also included in the scope of the compounds and preparing methods according to the present invention.

For example, the derivative of the present invention in which R₅ is hydrogen in Chemical Formula 1′ can be prepared by oxidizing 3-mercapto-6-methylmercaptopiperazinedione represented by Chemical Formula 2. At this time, the oxidation reaction may be performed using a reaction known in the art without limitation. Preferably iodine (I₂) or DDQ (2,3-dichloro-5,6-dicyano-1,4-benzoquinone) may be used, but is not limited thereto.

In Chemical Formula 1′, the derivatives in which R₅ is not hydrogen can be prepared by treating a compound of Chemical Formula 1′ in which R₅ is hydrogen with an appropriate electrophiles.

Another embodiment of the present invention also provides a method of preparing a 3-mercapto-6-methylmercaptopiperazinedione derivative represented by Chemical Formula 2, comprising reducing an intermediate represented by Chemical Formula 3 having a ring structure containing 3 to 4 sulfur atoms to form two thiol groups of Chemical Formula 2.

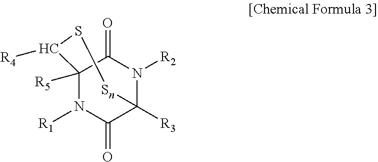

(In the Chemical Formula 3, R₁, R₂, R₃, and R₄ are the same as in Chemical Formula 1 above; R₅ is H; and n is 2 or 3.)

At this time, the reduction reaction may be performed using a reaction known in the art without any limitation. It may be preferably carried out using a hydride-based reducing agent such as sodium borohydride or lithium borohydride. More preferably, it may be performed using sodium borohydride, but is not limited thereto.

The other embodiment of the present invention provides a method for preparing the compound represented by Chemical Formula 3, comprising reacting a 6-(1-hydroxyalkyl)piperazin-2,5-dione derivative represented by Chemical Formula 4 (wherein R₅ =hydrogen) with (a) sulfur (S₈) and (b) lithium bis(trimethylsilyl)amide (LiHMDS) or sodium bis(trimethylsilyl)amide (NaHMDS).

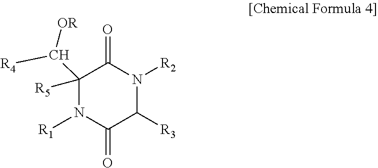

(In the Chemical Formula 4, R₁, R₂, R₃, and R₄ are the same as in Chemical Formula 1 above; R₅ is H; and R is a protecting group.)

In Chemical Formula 4, R is a protecting group, and a hydroxyl protecting group known in the art may be used as the R without limitation. Preferably, silicon-based protecting groups such as TBDMS (t-Bu(Me)₂silyl) and TMS (trimethylsilyl); ether-based protecting groups such as t-Bu; ester-based protecting groups such as acetyl; and a protecting group such as dihydropyran may be used, but is not limited thereto.

In the case of general 3,6-substituted-piperazinedione, when reacted with sulfur under strong alkali conditions, a ring structure in which sulfur atoms are directly bonded to the 3,6-position is formed, whereas in the case of Chemical Formula 4, the hydroxymethyl group is first eliminated under strong alkali conditions, and then an intermediate of highly reactive α,β-unsaturated ketone is generated, and this intermediate immediately reacts with sulfur to form a unique ring structure with a methylene bridge like Chemical Formula 3. At this time, the number of sulfur atoms forming the ring structure is 3-4, which varies depending on the structure of the starting material or reaction conditions (see mechanism below).

[Mechanism of Formation of Chemical Formula 3 from Chemical Formula 4]

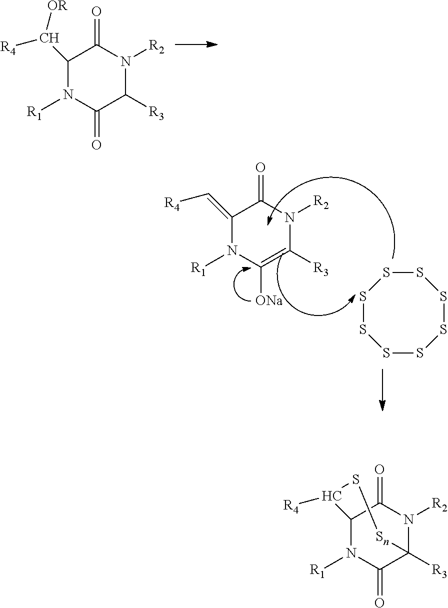

One embodiment of the present invention also provides a method for preparing a compound represented by the following Chemical Formula 1′, comprising
(S1) reacting a compound represented by Chemical Formula 4 with (a) sulfur (S₈) and (b) LiHMDS (lithium bis(trimethylsilyl)amide) or NaHMDS (sodium bis(trimethylsilyl)amide) to obtain a compound represented by Chemical Formula 3;
(S2) reducing the compound of Chemical Formula 3 to obtain a compound represented by Chemical Formula 2; and
(S3) forming an intramolecular disulfide crosslink from the compound represented by Chemical Formula 2.

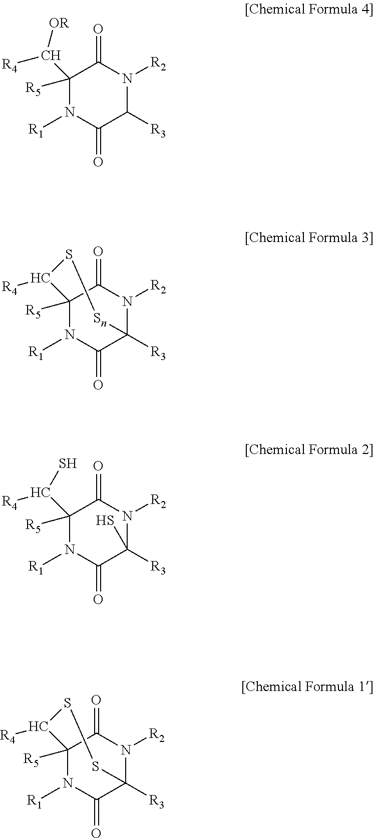

In the Chemical Formula 4, 3, 2 and 1′, R₁, R₂, R₃, and R₄ are the same as in Chemical Formula 1 above; R₅ is H; R is a protecting group; and n is 2 or 3.

As the reaction reagents, reagents mentioned in the above-mentioned methods may be used in this embodiment.

### Advantageous Effects of Invention

The present disclosure provides a piperazinedione compound containing a —CH₂—S—(S)ₙ— bridge and pharmaceutically acceptable salts thereof. The compounds of the present disclosure include intramolecular disulfide crosslinking to improve cell permeability and mimic the function of 2-Cys-Prx by being rapidly reduced to a compound having two thiol groups in cells. In particular, the compounds of the present disclosure inhibit PDGF-induced migration and proliferation of vascular smooth muscle cells by mimicking the function of the PrxII isoform in arterial vascular cells, thereby inhibiting intimal thickening, while the compounds of the present disclosure promote VEGF-induced migration and proliferation of vascular endothelial cells, thereby enhancing re-endothelialization. Therefore, the compound according to the present disclosure and its pharmaceutically acceptable salt can find their use in a pharmaceutical composition for preventing or treating vascular diseases. In particular, the compound or salt thereof of the present disclosure is useful for the treatment or prevention of vascular diseases such as ischemic coronary artery disease, arteriosclerosis, vascular restenosis, and pulmonary arterial hypertension.

### BRIEF DESCRIPTION OF DRAWINGS

FIGS. 1-3 are the results of immunoblot analysis about the effect of the compounds according to the present disclosure on PDGF-induced tyrosine phosphorylation in HASMC and PASMC depleted of PrxII. FIG. 1 is the results of evaluating the con-centration-dependent effect of of Compound 1 on the degree of Tyr 857 phosphorylation of the PDGF receptor-β (PDGFRβ) augmented by PrxII knock-down in human aortic smooth muscle cells (HASMC). FIG. 2 is the results of evaluating the effect of Compound 1, 2, 5, 6, 10, 15, 19, 20, 21, 22, and 23 on the degree of Tyr 857 phosphorylation of PDGF receptor-β augmented by PrxII knock-down in human aortic smooth muscle cells. FIG. 3 is the results of evaluating the effect of Compound 8 on the degree of the PDGF-induced intracellular tyrosine phosphorylation augmented by PrxII knock-down and the degree of phosphorylation of signaling proteins such as PLC-γ1 at downstream of the PDGF receptor-β in human pulmonary artery smooth muscle cells (PASMC).

FIGS. 4-6 are the results of immunoblot analysis of the effect of the compound according to the present disclosure on VEGF-induced tyrosine phosphorylation in HAEC and PAEC depleted of PrxII. FIG. 4 is the results of evaluating the effect of each concentration of Compound 1 on the degree of Tyr 1175 phosphorylation of the VEGF receptor-2 (VEGFR2) augmented by PrxII knock-down in HAECs. FIG. 5 is the results of evaluating the effect of Compound 1, 2, 5, 6, 10, 20, 21, and 23 on the degree of VEGF receptor phosphorylation augmented by PrxII knock-down in HAECs. FIG. 6 is the results of evaluating the effect of Compound 8 on the degree of VEGF-induced intracellular tyrosine phosphorylation augmented by PrxII knock-down and the degree of phosphorylation of signaling proteins such as ERK2 at downstream of the VEGF receptor-2 in PAECs.

FIGS. 7-9 are the results of preclinical efficacy tests using a Sugen/hypoxia rat model of pulmonary arterial hypertension. FIG. 7 shows significant reduction of right ventricular systolic pressure and inhibition of right ventricular hypertrophy compared to vehicle control group. FIG. 8 is a result showing that occluded blood vessels in the vehicle control group were reversed. FIG. 9 is the immunofluorescence staining results showing the proliferation of pulmonary arterial endothelial cells and the inhibition of smooth muscle cell hyperplasia.

### MODE FOR THE INVENTION

Hereinafter, the present invention is described in considerable detail with examples to help those skilled in the art understand the present invention. However, the following examples are offered by way of illustration and are not intended to limit the scope of the invention. It is apparent that various changes may be made without departing from the spirit and scope of the invention or sacrificing all of its material ad-vantages.

Amino acids used as starting materials in the following preparations were L-form, which was easy to obtain. However, it would be okay to proceed with synthesis using D-form or racemic form.

### Preparation example 1: Synthesis of 1,6,8-trimethyl-2,3-dithia-6,8-diazabicyclo[3.2.2]nonane-7,9-dione (Compound 1)

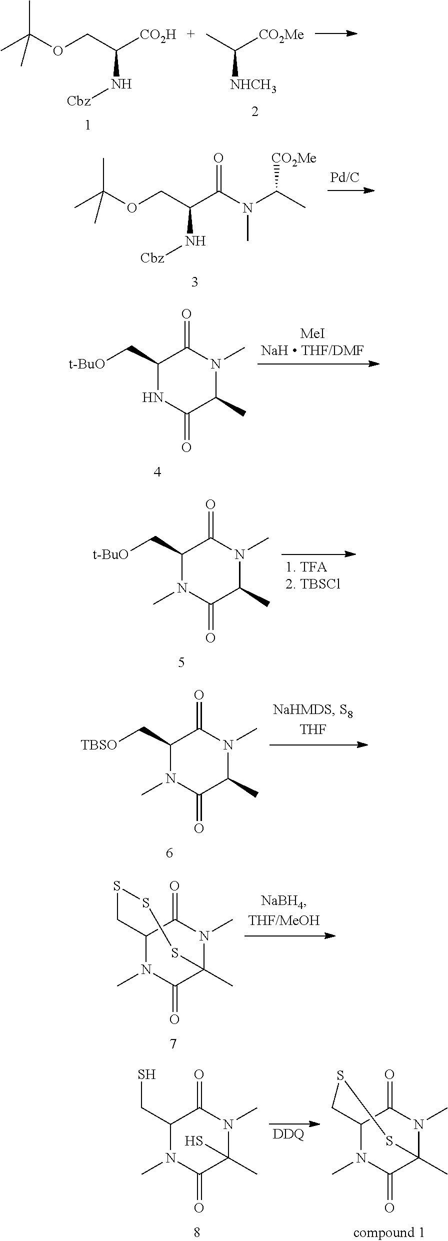

### Step 1. Methyl N—(N-benzyloxycarbonyl)-O-t-butyl-L-seryl)-N-methyl-L-alanine (intermediate 3)

To a solution of intermediate 2 (8.32 g, 54.2 mmol, 1.00 eq) in DMF (100 mL) was added DIPEA (35.0 g, 271 mmol, 47.2 mL, 5.00 eq), intermediate 1 (16.0 g, 54.2 mmol, 1.00 eq) and HATU (30.9 g, 81.3 mmol, 1.50 eq) and the reaction mixture was stirred at 25° C. for 12 h. Water (1000 mL) was added to the reaction mixture and extracted with ethyl acetate (100 mL*2). The combined organic layers were washed with brine (200 mL), dried over Na₂SO₄, filtered, and concentrated under reduced pressure to give a crude residue. This residue was purified by chromatography on silica to give intermediate 3 (20.0 g, 48.2 mmol, 88.9% yield) as yellow oil. MS (ESI) m/z=395.1 [M+1]⁺

### Step 2. (3S,6S)-3-(t-butoxymethyl)-1,6-dimethylpiperazine-2,5-dione (intermediate 4)

To a mixture of intermediate 3 (35.0 g, 88.7 mmol, 1.00 eq) in MeOH (450 mL) was added Pd/C (3.00 g, 10.0% purity). The mixture was stirred at 50° C. under H₂ (50 Psi) for 12 hours. After filtering the mixture, the filtrate was concentrated under reduced pressure to give intermediate 4 (15.0 g, 65.7 mmol, 74.1% yield) as light-yellow oil.

### Step 3. (3S,6S)-3-(t-butoxymethyl)-1,4,6-trimethylpiperazine-2,5-dione (intermediate 5)

To a stirred solution of intermediate 4 (19.0 g, 83.2 mmol, 1.00 eq) in DMF (500 mL) at 0° C. was added NaH (3.99 g, 99.9 mmol, 60% purity, 1.20 eq) and Mel (186 g, 1.31 mmol, 81.4 mL, 15.7 eq). The resulting solution was stirred for 12 hours under a nitrogen atmosphere. Water (2 L) was added to the reaction and the mixture was extracted with CH₂Cl₂ (300 mL*3). The combined organic phases were washed with brine (500 mL*2), dried over anhydrous Na₂SO₄, filtered, and concentrated in vacuo. This product was purified on silica to give intermediate 5 (19.0 g, 78.4 mmol, 94.2% yield) as light-yellow oil.

### Step 4. (3S,6S)-3-(((t-butyldimethylsilyl)oxy)methyl)-1,4,6-trimethylpiperazine-2,5-dione (intermediate 6)

To a mixture of intermediate 5 (19.0 g, 78.4 mmol, 1.00 eq) in CH₂Cl₂ (50.0 mL) was added TFA (77.0 g, 675 mmol, 50.0 mL, 8.61 eq) and stirred at 25° C. for 12 hours. The reaction mixture was concentrated under reduced pressure, then the concentrate was dissolved in DMF (200 mL). To this mixture was added TBSCl (14.6 g, 96.7 mmol, 11.9 mL, 1.20 eq) and imidazole (16.5 g, 242 mmol, 3.00 eq) and stirred at 25° C. for 1 hour. Water (1000 mL) was added to the reaction mixture and the mixture was extracted with CH₂Cl₂ (300 mL*3). The combined organic phases were washed with brine (200 mL*2), dried over anhydrous Na₂SO₄, filtered, and concentrated in vacuo. The residue was purified on silica gel to give intermediate 6 (20.0 g, 66.6 mmol, 82.6% yield) as light-yellow solid.

### Step 5. 1,6,8-Trimethyl-2,3-dithia-6,8-diazabicyclo[3.2.2]nonane-7,9-dione (Compound 1)

Sulfur (853 mg, 26.63 mmol, 8 eq) was added to anhydrous THF (53 mL) at room temperature, then NaHMDS (10 mL, 1M in THF, 10 mmol) was slowly added dropwise, followed by stirring for 10 minutes. Intermediate 6 (1 g, 3.33 mmol, 1 eq) dissolved in THF (12 mL) was added dropwise at room temperature for 10 minutes, and then additional NaHMDS (8.3 mL, 1M in THF, 8.3 mmol) was slowly added dropwise. After the reaction was stirred for 75 minutes, ammonium chloride solution (200 mL) was added to the reaction mixture and extracted with CH₂Cl₂ (200 mL*2). The combined organic phases were washed with brine (100 mL*2), dried over anhydrous Na₂SO₄, filtered, and concentrated in vacuo. Intermediate 7 (trisulfide ring) obtained by purifying the residue on silica gel was used in the next step.

Intermediate 7 (200 mg, 0.64 mmol) was dissolved in THF (10 mL) and MeOH (10 mL) at room temperature then cooled to 0° C. After NaBH₄ (0.12 g, 3.2 mmol, 5 eq) was slowly added dropwise at 0° C., the temperature was raised to room temperature and the mixture was stirred for 30 minutes. The reaction mixture was concentrated under reduced pressure. Water (10 mL) and aqueous NaHCO₃ solution (40 mL) were added to the reaction mixture and stirred at 0° C. The mixture was washed with CH₂Cl₂ (100 mL*3) to remove impurities. The aqueous layer was acidified with 1N aqueous hy-drochloric acid and then extracted with CH₂Cl₂ (200 mL*3). The combined organic phases were washed with brine (100 mL*2), dried over anhydrous Na₂SO₄, filtered, and partially concentrated in vacuo to 100 mL to give intermediate 8 which was used directly in the next step without purification.

In another reaction vessel, 2,3-dichloro-5,6-dicyano-1,4-benzoquinone (DDQ, 120 mg, 0.8 eq) was dissolved in THF (10 mL) and CH₂Cl₂ (100 mL). The solution of intermediate 8 obtained above was slowly added dropwise thereto and reacted at room temperature for 30 minutes. To the reaction mixture was added aqueous NaHCO₃ (200 mL) and the mixture was extracted with CH₂Cl₂ (200 mL*3). The combined organic phases were washed with brine (200 mL*2), dried over anhydrous Na₂SO₄, filtered, and concentrated in vacuo. The residue was purified on silica gel to give 101 mg of compound 1 as pale-yellow solid.

¹H NMR (400 MHz, CDCl₃) δ 4.29 (s, 1H), 3.09 (s, 3H), 3.02 (s, 3H), 2.82 (dd, J=12.8, 7.5 Hz, 2H), 1.86 (s, 3H).

¹³C NMR (101 MHz, CDCl₃) δ 168.79 (s), 166.79 (s), 70.19 (s), 63.49 (s), 33.52 (s), 28.56 (s), 28.13 (s), 21.43 (s).

### Preparation example 2: Synthesis of 6-benzyl-1,8-dimethyl-2,3-dithia-6,8-diazabicyclo[3.2.2]nonane-7,9-dione (Compound 2)

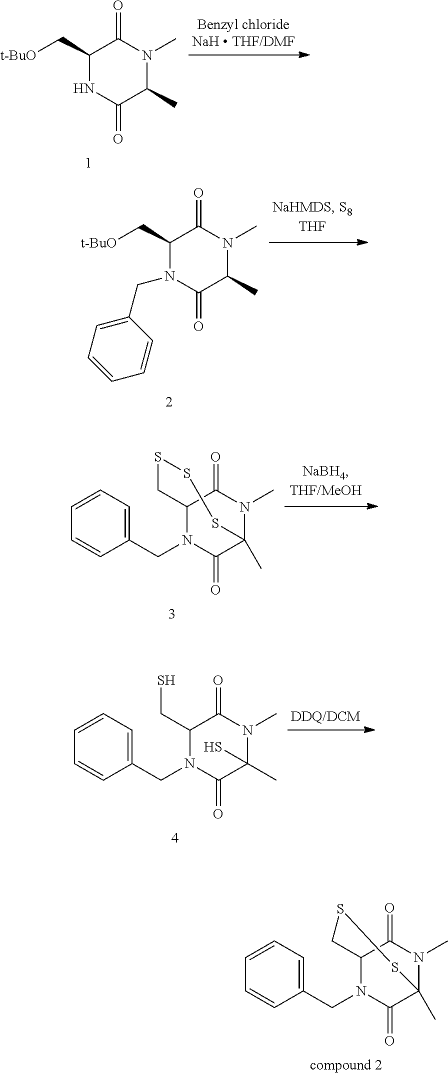

### Step 1. (3S,6S)-1-Benzyl-6-(t-butoxymethyl)-3,4-dimethylpiperazine-2,5-dione (intermediate 2)

It was synthesized from intermediate 1 (the same material as intermediate 4 in preparation example 1) in a similar manner to the method for preparing intermediate 5 of preparation example 1. However, benzyl bromide was used instead of iodomethane.

### Step 2. 6-Benzyl-1,8-dimethyl-2,3-dithia-6,8-diazabicyclo[3.2.2]nonane-7,9-dione (Compound 2)

It was prepared by a similar synthesis method to compound 1 of preparation example 1. That is, 1 g of intermediate 3 was obtained using intermediate 2 (1.9 g, 5.97 mmol), sulfur (1.53 g, 8 eq), and NaHMDS (29.8 mL, 1M in THF, 29.8 mmol). Then, intermediate 4 was obtained by using intermediate 3 (0.8 g, 2.35 mmol) and NaBH4 (0.44 g, 5 eq), and then used in the next reaction without purification. Thus, compound 2 (0.44 g) was obtained using intermediate 4 and DDQ (0.426 g, 0.8 eq).

¹H NMR (400 MHz, CDCl₃) δ 7.37-7.29 (m, 9H), 7.26 (t, J=7.8 Hz, 8H), 5.20 (d, J=14.9 Hz, 1H), 4.75 (d, J=14.7 Hz, 3H), 4.47 (d, J=14.8 Hz, 3H), 4.30 (d, J=4.8 Hz, 3H), 4.01 (d, J=14.8 Hz, 1H), 3.88 (d, J=14.3 Hz, 1H), 3.32 (dd, J=14.4, 6.4 Hz, 1H), 3.09 (s, 8H), 2.98 (s, 2H), 2.59 (dt, J=23.7, 9.9 Hz, 6H), 1.93 (s, 2H), 1.91 (s, 8H)

¹³C NMR (101 MHz, CDCl₃) δ 168.80 (s), 166.95 (s), 135.17 (s), 129.24 (s), 128.69 (s), 128.48 (s), 69.81 (s), 61.02 (s), 49.67 (s), 28.55 (d, J=7.5 Hz), 21.61 (s)

### Preparation example 3: Synthesis of 1,8-dimethyl-6-(3,4,5-trimethoxybenzyl)-2,3-dithia-6,8-diazabicyclo[3.2.2]nonane-7,9-dione (Compound 3)

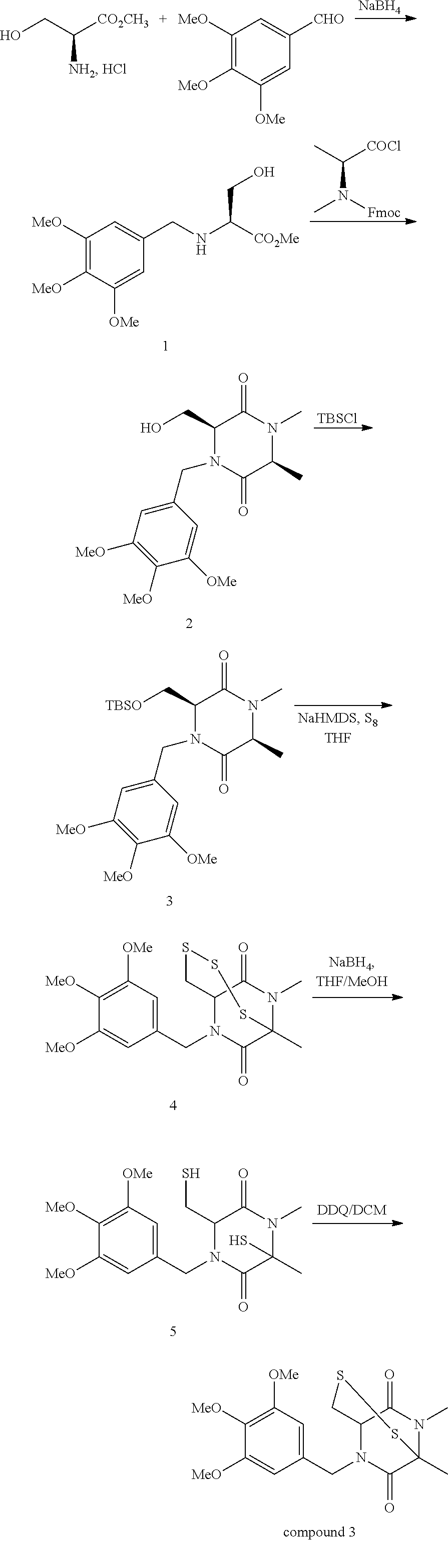

### Step 1. Methyl-(3,4,5-trimethoxybenzyl)-L-serine (intermediate 1)

3,4,5-trimethoxybenzaldehyde (3.25 g, 1 eq) was added a to a solution of serine methyl ester (5 g, 32.1 mmol) in MeOH (50 mL), and cooled to 0° C. After adding Et₃N (4.48 mL, 1 eq) and stirring for 2 hours, NaBH₄ (2.41 g, 2 eq) was added dropwise at 0° C. and stirred for 2 hours. The solvent was removed under reduced pressure after cooling the reactant to room temperature, then quenched with brine. The reaction mixture was extracted with CH₂Cl₂, the organic layer was washed with brine and dried over anhydrous Na₂SO₄. After removing the solvent under reduced pressure, the obtained residue was purified on silica to give intermediate 1 (2.41 g).

### Step 2. (3S,6S)-3-(hydroxymethyl)-1,6-dimethyl-4-(3,4,5-trimethoxybenzyl)piperazine-2,5-di one (intermediate 2)

To intermediate 1 (2.41 g, 8.1 mmol) in CH₂Cl₂ (150 mL) was added NaHCO₃ (1.0 g, 1.5 eq) dissolved in a small amount of water at room temperature. Fmoc-N-Me-alanine chloride (2.77 g, 1 eq) dissolved in CH₂Cl₂ (40 mL) was added to the intermediate 1 solution, and stirred at room temperature for 3 hours. After adding water (100 mL) to the reaction vessel, the organic layer was washed with 1N HCl and brine, and then the organic layer was concentrated under reduced pressure. EtOH (150 mL) and piperidine (0.8 mL, 8.1 mmol, 1 eq) were added to the obtained product, and the mixture was stirred for 4 hours while refluxing. After cooling the reaction mixture to room temperature, the organic solvent was concentrated under reduced pressure, and the residue was purified on silica to obtain intermediate 2 (0.92 g).

### Step 3. (3S,6S)-3-(((t-butyldimethylsilyl)oxy)methyl)-1,6-dimethyl-4-(3,4,5-trimethoxybenzyl)piperazine-2, 5-dione (intermediate 3)

To a stirred solution of intermediate 2 (0.91 g, 2.58 mmol) in anhydrous CH₂Cl₂ (20 mL) was added tert-butyldimethylsilyl chloride (TBSCl, 0.78 g, 2 eq) at 0° C. under nitrogen atmosphere. To this was slowly added a solution of imidazole (0.35 g, 2 eq) in anhydrous CH₂Cl₂ (5 mL). The mixture was stirred at 20° C. for 12 h then quenched with water (20 mL). The organic layer was washed twice with water and brine (100 mL) and dried over anhydrous Na₂SO₄. The organic layer was concentrated, the solvent was completely removed under reduced pressure, and the resulting material was purified on silica to obtain intermediate 3 (1.21 g).

### Step 4. 1,8-Dimethyl-6-(3,4,5-trimethoxybenzyl)-2,3-dithia-6,8-diazabicyclo[3.2.2]nonane-7,9-dione (Compound 3)

It was prepared by a similar synthesis method to compound 1 of preparation example 1. That is, 0.53 g of intermediate 4 was obtained using intermediate 3 (1.2 g, 2.57 mmol), sulfur (0.66 g, 8 eq), and NaHMDS (14.1 mL, 1M in THF, 14.1 mmol). Intermediate 5 was obtained by using intermediate 4 (0.25 g, 0.58 mmol) and NaBH₄ (0.1 g, 5 eq), and then used in the next reaction without purification. Thus, compound 3 (0.125 g) was obtained using intermediate 5 and DDQ (0.094 g, 0.42 mmol, 0.8 eq).

¹H NMR (400 MHz, CDCl₃) δ 6.48 (s, 2H), 4.65 (d, J=14.7 Hz, 1H), 4.43 (d, J=14.7 Hz, 1H), 4.35 (dd, J=5.7, 2.0 Hz, 1H), 3.84 (s, 6H), 3.83 (s, 3H), 3.10 (s, 3H), 2.57 (qd, J=14.0, 3.9 Hz, 2H), 1.91 (s, 3H)

¹³C NMR (101 MHz, CDCl₃) δ 168.85, 166.98, 153.82, 138.07, 130.98, 105.72, 69.85, 61.53, 61.05, 56.40, 50.22, 28.62, 28.39, 21.60

### Preparation example 4: synthesis of 6-(3,5-difluorobenzyl)-1,8-dimethyl-2,3-dithia-6,8-diazabicyclo[3.2.2]nonane-7,9-dion e (Compound 4)

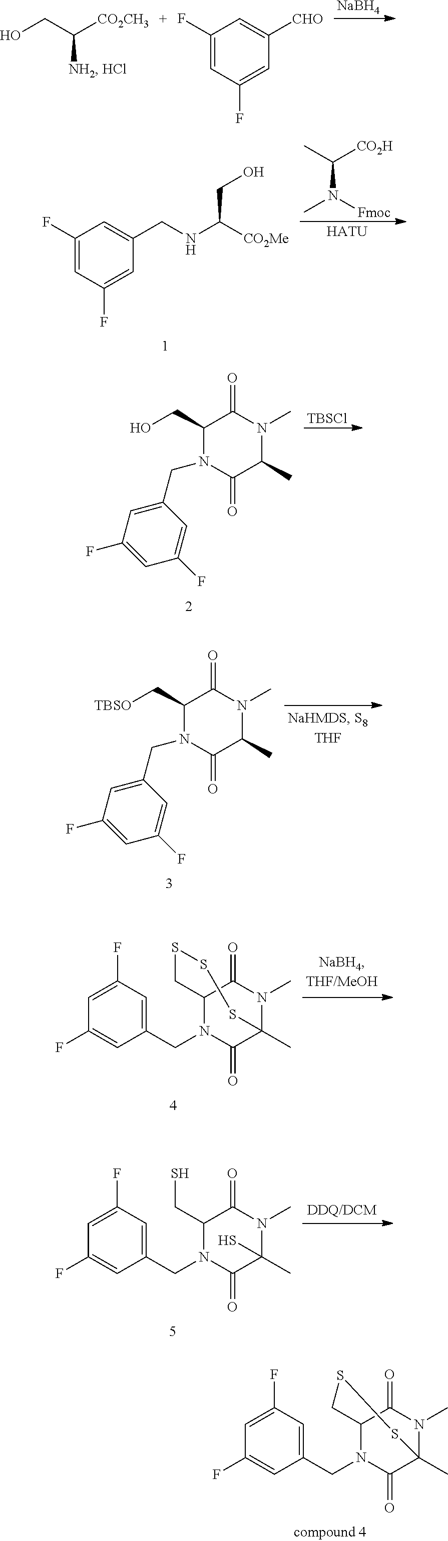

### Step 1. Methyl-(3,5-difluorobenzyl)-L-serine (intermediate 1)

It was prepared by a similar synthesis method to intermediate 1 of preparation example 3. However, 2,4-difluorobenzaldehyde was used instead of 3,4,5-trimethoxybenzaldehyde.

### Step 2. (3S,6S)-1-(3,5-difluorobenzyl)-6-(hydroxymethyl)-3,4-dimethylpiperazine-2,5-dione (intermediate 2)

It was prepared by a similar synthesis method to intermediate 2 of preparation example 3. However, Fmoc-N-Me-alanine was used instead of Fmoc-N-Me-alanine chloride.

### Step 3. (3S,6S)-3-(((t-butyldimethylsilyl)oxy)methyl)-4-(3,5-difluorobenzyl)-1,6-dimethylpipe razine-2,5-dione (intermediate 3)

It was prepared by a similar synthesis method to intermediate 3 of preparation example 3.

### Step 4. 6-(3,5-Difluorobenzyl)-1,8-dimethyl-2,3-dithia-6,8-diazabicyclo[3.2.2]nonane-7,9-dio ne (compound 4)

It was prepared by a similar synthesis method to compound 1 of preparation example 1. That is, 0.26 g of intermediate 4 was obtained using intermediate 3 (0.86 g, 2.08 mmol), sulfur (0.54 g, 8 eq), and NaHMDS (11.5 mL, 1M in THF, 11.5 mmol). Intermediate 5 was obtained by using intermediate 4 (0.26 g, 0.69 mmol) and NaBH₄ (0.13 g, 5 eq) and then used in the next reaction without purification. Thus, compound 4 (0.015 g) was obtained using the intermediate 5 and DDQ (0.13 g, 0.8 eq).

¹H NMR (400 MHz, CDCl₃) δ 7.37 (dd, J=15.0, 8.1 Hz, 1H), 6.86 (dt, J=20.7, 9.2 Hz, 2H), 4.77 (d, J=14.9 Hz, 1H), 4.44 (d, J=14.9 Hz, 1H), 4.37 (d, J=5.7 Hz, 1H), 3.08 (s, 3H), 2.75 (dd, J=14.1, 5.8 Hz, 1H), 2.67 (d, J=14.0 Hz, 1H), 1.88 (s, 3H)

¹³C NMR (101 MHz, CDCl₃) δ 168.62 (s), 167.03 (s), 164.21 (d, J=12.3 Hz), 162.46 (d, J=11.9 Hz), 161.71 (d, J=12.3 Hz), 159.99 (d, J=12.0 Hz), 132.44 (dd, J=9.7, 5.3 Hz), 118.46 (d, J=15.1 Hz), 112.38 (dd, J=21.3, 3.7 Hz), 104.52 (s), 104.27 (s), 104.01 (s), 69.8, 61.6, 42.7 (d, J=2.9 Hz), 28.8, 28.6, 21.5

### Preparation example 5: Synthesis of 1,8-dimethyl-6-(quinolin-2-ylmethyl)-2,3-dithia-6,8-diazabicyclo[3.2.2]nonane-7,9-dio ne (Compound 5)

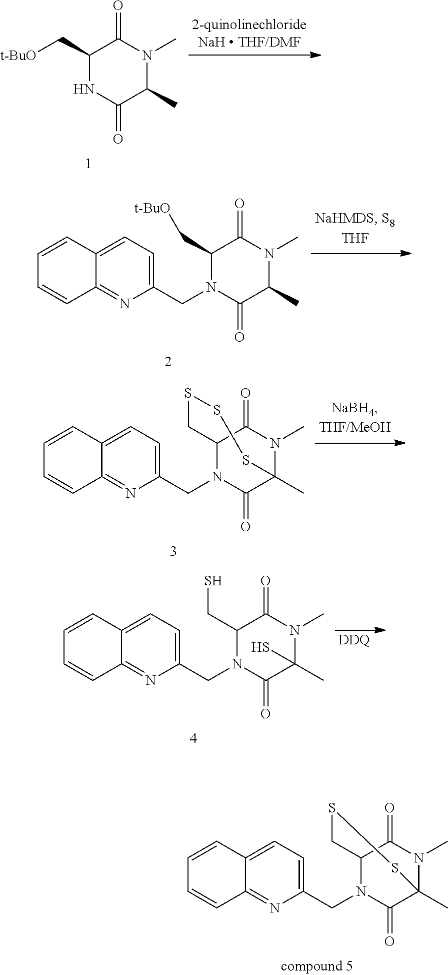

### Step 1. (3S,6S)-3-(t-butoxymethyl)-1,6-dimethyl-4-(quinolin-2-ylmethyl)piperazine-2,5-dione (intermediate 2)

It was synthesized from intermediate 1 (the same material as intermediate 4 in preparation example 1), in a similar manner to the method for preparing intermediate 2 of preparation example 2. However, 2-quinolinemethyl chloride was used instead of iodomethane.

### Step 2. 1,8-Dimethyl-6-(quinolin-2-ylmethyl)-2,3-dithia-6,8-diazabicyclo[3.2.2]nonane-7,9-di one (compound 5)

It was prepared by a similar synthesis method to compound 1 of preparation example 1. That is, intermediate 2 (1.15 g, 3.11 mmol), sulfur (0.79 g, 8 eq), and NaHMDS (15.6 mL, 1M in THF, 15.6 mmol) was used to obtain 0.72 g (59% yield) of intermediate 3. Intermediate 4 was obtained by using intermediate 3 (0.55 g, 1.44 mmol) and NaBH₄ (0.26 g, 5 eq), and then used in the next reaction without purification. Thus, compound 5 (0.25 g) was obtained using intermediate 4 and DDQ (0.255 g, 0.8 eq).

¹H NMR (400 MHz, CDCl₃) δ 8.16 (d, J=8.4 Hz, 1H), 8.02 (dd, J=8.4, 0.7 Hz, 1H), 7.81 (dd, J=8.1, 1.3 Hz, 1H), 7.72 (ddd, J=8.4, 6.9, 1.4 Hz, 1H), 7.55 (ddd, J=8.1, 6.9, 1.2 Hz, 1H), 7.45 (d, J=8.4 Hz, 1H), 5.06 (d, J=14.9 Hz, 1H), 4.71 (d, J=14.9H, 1H), 4.61 (dd, J=6.1, 1.8 Hz, 1H), 3.11 (s, 3H), 2.81 (dd, J=14.0, 1.8 Hz, 1H), 2.71 (dd, J=14.0, 6.1 Hz, 1H), 1.91 (s, 3H)

¹³C NMR (101 MHz, CDCl₃) δ 168.9, 167.1, 155.8, 147.7, 137.6, 130.1, 129.3, 127.8, 127.7, 127.0, 120.7, 69.8, 62.0, 52.2, 28.8, 28.6, 21.6

### Preparation example 6: Synthesis of 1,8-dimethyl-6-(pyridin-2-ylmethyl)-2,3-dithia-6,8-diazabicyclo[3.2.2]nonane-7,9-dion e (Compound 6)

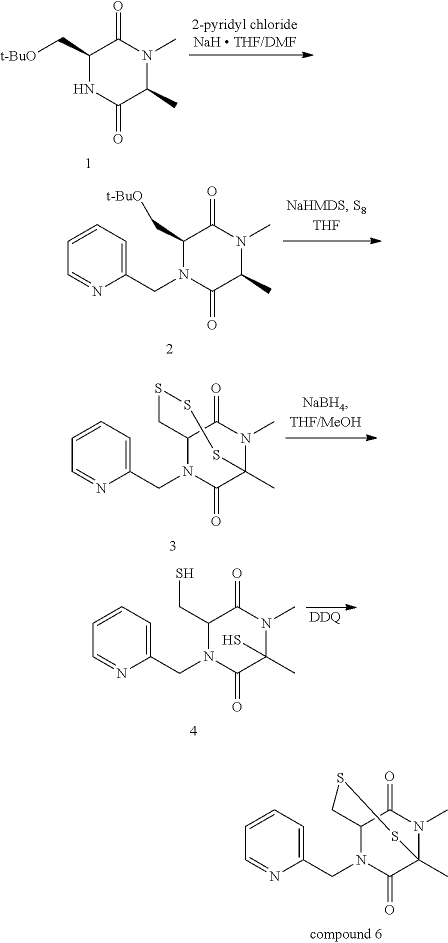

### Step 1. (3S,6S)-3-(t-butoxymethyl)-1,6-dimethyl-4-(pyridin-2-ylmethyl)piperazine-2,5-dione (intermediate 2)

It was synthesized from intermediate 1 (the same material as intermediate 4 in preparation example 1), in a similar manner to the method for preparing intermediate 2 of preparation example 2. However, 2-pyridinemethyl chloride was used instead of benzyl bromide.

### Step 2. 1,8-Dimethyl-6-(pyridin-2-ylmethyl)-2,3-dithia-6,8-diazabicyclo[3.2.2]nonane-7,9-dio ne (Compound 6)

It was prepared by a similar synthesis method to compound 1 of preparation example 1. That is, 0.68 g of intermediate 3 was obtained using intermediate 2 (1.05 g, 3.29 mmol), sulfur (0.84 g, 8 eq), and NaHMDS (16.4 mL, 1M in THF, 16.4 mmol). Intermediate 4 was obtained by using intermediate 3 (0.58 g, 1.70 mmol) and NaBH4 (0.32 g, 5 eq), and then used in the next reaction without purification. Thus, compound 6 (0.23 g) was obtained using intermediate 4 and DDQ (0.34 g, 0.8 eq).

¹H NMR (400 MHz, CDCl₃) δ 8.52 (ddd, J=4.9, 1.7, 0.9 Hz, 1H), 7.67 (td, J=7.7, 1.8 Hz, 1H), 7.33 (d, J=7.8 Hz, 1H), 7.22 (ddd, J=7.5, 4.9, 1.1 Hz, 1H), 4.92 (d, J=14.9 Hz, 1H), 4.56 (dd, J=6.0, 1.9 Hz, 1H), 4.47 (d, J=14.9 Hz, 1H), 3.09 (s, 3H), 2.81 (dd, J=14.0, 1.9 Hz, 1H), 2.72 (dd, J=14.0, 6.0 Hz, 1H), 1.87 (s, 3H)

¹³C NMR (101 MHz, CDCl₃) δ 168.88, 166.95, 155.48, 149.68, 137.33, 123.24, 123.22, 69.81, 61.94, 51.56, 28.70, 28.56, 21.52

### Preparation example 7: Synthesis of 11-benzyltetrahydro-5H,7H-4,9a-(epiminomethano)pyrrolo[2,1-c][1,2,4]dithiazepine-5,10-dione (Compound 7)

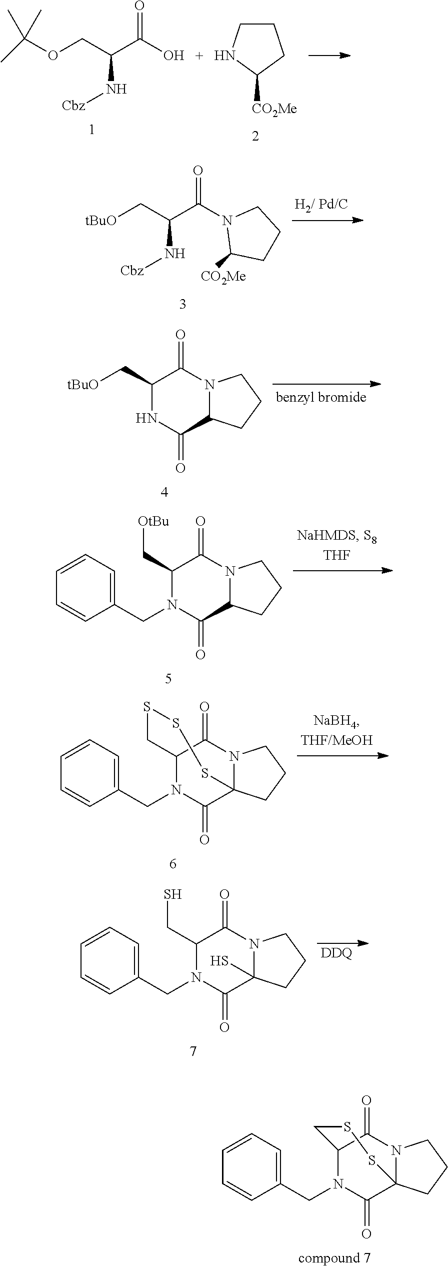

### Step 1. Methyl N-((benzyloxycarbonyl)-O-(t-butyl)-L-seryl-L-proline (intermediate 3)

It was prepared similarly to the synthesis of intermediate 3 in preparation example 1. However, proline methyl ester was used instead of N-methylalanine methyl ester.

### Step 2. (3S,8aR)-3-(t-butoxymethyl)hexahydropyrrolo[1,2-a]pyrazine-1,4-dione (intermediate 4)

To a mixture of intermediate 3 (98.0 g, 1.00 eq) in MeOH (980 mL) was added Pd/C (9.80 g, 10% purity) and the mixture was stirred under H₂ (50 psi) at 70° C. for 12 h. After filtration, the filtrate was concentrated. The residue was purified by column chromatography to give intermediate 4 (35.0 g, 59.8% yield, 99.0% purity) as white solid.

### Step 3. (3S,8aR)-2-benzyl-3-(t-butoxymethyl)hexahydropyrrolo[1,2-a]pyrazine-1,4-dione (intermediate 5)

It was synthesized similarly to the method for preparing intermediate 2 of preparation example 2.

### Step 4. 11-Benzyltetrahydro-5H,7H-4,9a-(epiminomethano)pyrrolo[2,1-c][1,2,4]dithiazepine-5,10-dione (Compound 7)

It was prepared by a similar synthesis method to compound 1 of preparation example 1. That is, 0.43 g of intermediate 6 was obtained using intermediate 5 (1.5 g, 4.54 mmol), sulfur (1.16 g, 8 eq), and NaHMDS (22.7 mL, 1M in THF, 22.7 mmol). Intermediate 7 was obtained by using intermediate 6 (0.41 g, 1.16 mmol) and NaBH4 (0.22 g, 5 eq) and then used in the next reaction without purification. Thus, compound 7 (0.085 g) was obtained using intermediate 7 and DDQ (0.24 g, 0.8 eq).

¹H NMR (400 MHz, CDCl₃) δ 7.40-7.26 (m, 3H), 7.25 (d, J=8.6 Hz, 2H), 4.64 (d, J=14.8 Hz, 1H), 4.51 (d, J=14.8 Hz, 1H), 4.23 (d, J=6.0 Hz, 1H), 3.87-3.79 (m, 1H), 3.73 (dd, J=19.0, 9.8 Hz, 1H), 2.69 (ddd, J=13.8, 11.7, 8.0 Hz, 1H), 2.60 (dd, J=14.1, 6.1 Hz, 1H), 2.52 (d, J=14.1 Hz, 1H), 2.33 (dd, J=14.0, 6.7 Hz, 1H), 2.17 (ddd, J=19.9, 11.7, 4.1 Hz, 2H)

¹³C NMR (101 MHz, CDCl₃) δ 167.98, 165.57, 135.31, 129.25, 128.71, 128.48, 72.95, 62.96, 49.29, 45.98, 35.16, 27.94, 21.19

### Preparation example 8: Synthesis of 12-benzyltetrahydro-5H,10H-4,10a-(epiminomethano)[1,4]oxazino[3,4-c][1,2,4]dithia zepine-5,11-dione (Compound 8)

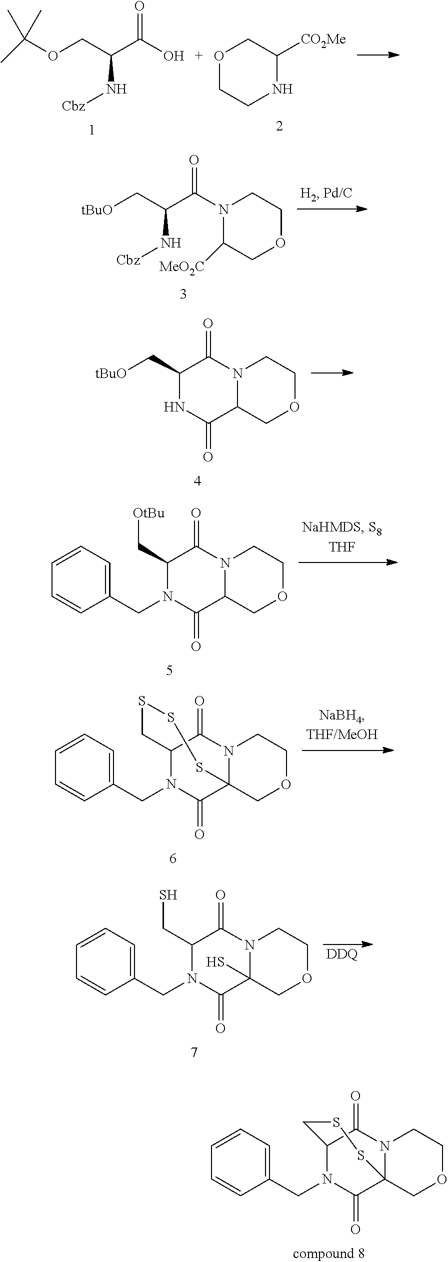

### Step 1. Methyl-4-(N-((benzyloxy)carbonyl)-O-(t-butyl)-L-seryl)morpholine-3-carboxylate (intermediate 3)

It was prepared similarly to the synthesis of intermediate 3 in preparation example 7. However, morpholine-3-carboxylic acid methyl ester was used instead of proline methyl ester.

### Step 2. (7S)-7-(t-butoxymethyl)hexahydropyrazino[2,1-c][1.4]oxazin-6,9-dione (intermediate 4)

It was prepared similarly to the synthesis of intermediate 4 in preparation example 7.

### Step 3. (7S)-8-Benzyl-7-(t-butoxymethyl)hexahydropyrazino[2,1-c][1.4]oxazine-6,9-dione (intermediate 5)

It was prepared similarly to the synthesis of intermediate 5 in preparation example 7. Two isomers (diastereomer) were obtained, with one of two isomers (5.20 g, 22.4% yield, 99.3% purity) being white solid while another isomer (5.10 g, 21.8% yield, 98.6% purity) being yellow oil.

HPLC: 99.3% purity (isomer 1) & 98.6% purity (isomer 2)

H NMR (isomer 1): 400 MHz, CDCl₃ δ 7.19-7.44 (m, 5H), 5.33 (d, J=15.2 Hz, 1H), 4.50 (dd, J=13.6, 2.0 Hz, 1H), 4.25-4.37 (m, 2H), 3.87-4.00 (m, 3H), 3.71-3.84 (m, 2H), 3.61-3.69 (m, 1H), 3.47 (td, J=11.6, 2.4 Hz, 1H), 2.88 (td, J=12.8, 3.6 Hz, 1H), 1.17 ppm (s, 9H).

H NMR (isomer 2): 400 MHz, CDCl₃ δ7.17-7.43 (m, 5H), 5.21 (d, J=15.2 Hz, 1H), 4.48 (dd, J=12.0, 4.2 Hz, 1H), 4.20-4.32 (m, 2H), 3.83-4.08 (m, 3H), 3.71 (dd, J=9.6, 1.6 Hz, 1H), 3.60 (dd, J=9.2, 2.4 Hz, 1H), 3.31-3.55 (m, 2H), 2.87 (td, J=12.8, 4.0 Hz, 1H), 1.11 ppm (s, 9H).

### Step 4. 12-benzyltetrahydro-5H,10H-4,10a-(epiminomethano[1,4]oxazino[3,4-c][1,2,4]dithiaz epine-5, 11-dione (Compound 8)

It was prepared by similar synthesis method to compound 1 of preparation example 1. That is, 1.24 g (58.3% yield) of intermediate 6 was prepared using intermediate 5 (any of the isomers, 2.0 g, 5.77 mmol), sulfur (1.48 g, 8 eq), and NaHMDS (28.9 mL, 1M in THF, 28.9 mmol). Intermediate 7 was obtained by using intermediate 6 (1.2 g, 3.26 mmol) and NaBH4 (0.62 g, 5 eq) and then used in the next reaction without purification. Thus, 0.45 g (41% yield) of compound 8 was obtained by using intermediate 7 and DDQ (0.67 g, 2.93 mmol, 0.8 eq).

¹H NMR (400 MHz, CDCl₃) δ 7.34 (t, J=7.9 Hz, 3H), 7.28-7.21 (m, 2H), 4.71 (d, J=14.8 Hz, 1H), 4.46 (d, J=14.8 Hz, 1H), 4.26 (d, J=3.5 Hz, 1H), 4.17 (d, J=13.7 Hz, 1H), 4.13-4.05 (m, 2H), 3.96 (d, J=13.5 Hz, 1H), 3.59 (td, J=12.3, 2.5 Hz, 1H), 3.21 (td, J=13.1, 4.0 Hz, 1H), 2.70-2.58 (m, 2H)

¹³C NMR (101 MHz, CDCl₃) δ 169.23, 165.90, 134.80, 129.33, 128.74, 128.74, 68.82, 65.81, 65.45, 60.51, 49.19, 38.20, 29.42

LCMS: RT=0.342 min, m/z=337.0 [M+H]⁺

### Preparation example 9: synthesis of 12-(pyridin-4-ylmethyl)tetrahydro-5H,10H-4,10a-(epiminomethano) [1,4]oxazino[3,4-c][1,2,4]dithiazepin-5,11-dione (Compound 9)

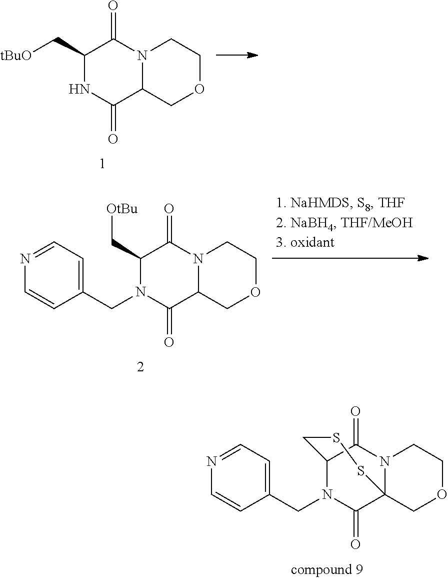 [SMILES: CC(C)(C)OC[C@@H]1NC(=O)C2COCCN2C1=O ; CC(C)(C)OC[C@H]1C(=O)N2CCOCC2C(=O)N1Cc1ccncc1 ; O=C1C2CSSC3(COCCN13)C(=O)N2Cc1ccncc1]

### Step 1. (7S)-7-(tert-butoxymethyl)-8-(pyridin-4-ylmethyl)hexahydropyrazino[2,1-c][1,4]oxazi ne-6,9-dione (intermediate 2)

It was synthesized from intermediate 1 (the same material as intermediate 4 in preparation example 8) in a similar manner to the method for preparing intermediate 5 of preparation example 8. However, 4-pyridylmethylchloride was used instead of benzyl bromide to give brown oil.

### Step 2. 12-(pyridin-4-ylmethyl)tetrahydro-5H,10H-4,10a-(epiminomethano)[1,4]oxazino[3,4-c][1,2,4]Dithiazepine-5,11-dione (Compound 9)

It was prepared by a similar synthesis method to compound 1 of preparation example 1. That is, 0.68 g of the trisulfide intermediate was obtained as a yellow solid from intermediate 2 (0.2 g, 0.58 mmol), sulfur (0.14 g, 8 eq), and NaHMDS (1.14 mL, 2M in THF, 5 eq). After reduction of trisulfide intermediate (0.68 g, 1.84 mmol) with NaBH₄ (0.2 g, 2.9 eq), it was used in the next reaction without purification. Thus, compound 9 (41 mg) was obtained as white solid using the reduced intermediate and iodine (0.47 g, 1.0 eq).

¹H NMR (400 MHz, CDCl₃) δ 2.66-2.73 (m, 2H) 3.17 (td, J=13.20, 4.38 Hz, 1H) 3.55 (td, J=12.26, 3.00 Hz, 1H) 3.89 (d, J=13.63 Hz, 1H) 4.00-4.13 (m, 3H) 4.16-4.37 (m, 2H) 4.75 (d, J=15.63 Hz, 1H) 7.11 (d, J=5.75 Hz, 2H) 8.43-8.68 (m, 2H)

LCMS: RT=0.342 min, m/z=337.9 [M+H]⁺

### Preparation example 10: Synthesis of 12-ethyltetrahydro-5H,10H-4,10a-(epiminomethano)[1,4]oxazino[3,4-c][1,2,4]dithiaze pine-5,11-dione (Compound 10)

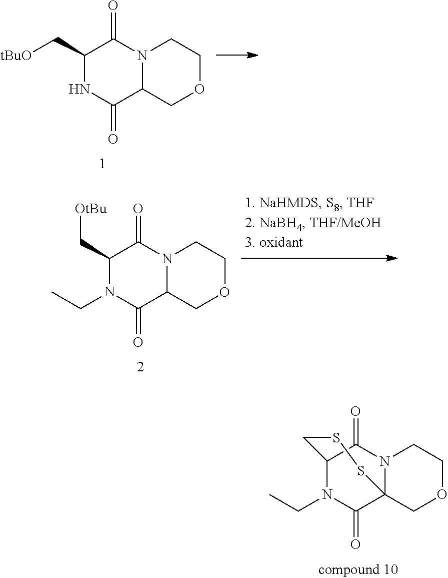 [SMILES: CC(C)(C)OC[C@@H]1NC(=O)C2COCCN2C1=O ; CCN1C(=O)C2COCCN2C(=O)[C@@H]1COC(C)(C)C ; CCN1C(=O)C23COCCN2C(=O)C1CSS3]

### Step 1. (7S)-7-(tert-butoxymethyl)-8-ethylhexahydropyrazino[2,1-c][1,4]oxazine-6,9-dione (intermediate 2)

It was synthesized from intermediate 1 (the same material as intermediate 4 in preparation example 8) in a similar manner to the method for preparing intermediate 5 of preparation example 8. However, ethyl bromide was used instead of benzyl bromide.

### Step 2. 12-ethyltetrahydro-5H,10H-4,10a-(epiminomethano)[1,4]oxazino[3,4-c][1,2,4]dithiaze pine-5, 11-dione (Compound 10)

It was prepared by a similar synthesis method to compound 1 of preparation example 1. That is, 0.35 g of trisulfide intermediate was obtained from intermediate 2 (0.57 g, 2.0 mmol), sulfur (0.51 g, 8 eq), and NaHMDS (5.0 mL, 2M in THF, 5 eq). After reduction of trisulfide intermediate (0.35 g, 1.2 mmol) with NaBH₄ (0.23 g, 5 eq), it was used in the next reaction without purification. Thus, compound 10 (130 mg) was obtained as white solid using the reduced intermediate and DDQ (0.24 g, 0.9 eq).

¹H NMR (400 MHz, DMSO-_{d6}) δ 1.07 (t, 3H), 2.95 (m, 1H), 3.00 (t, 2H), 3.13 (m, 1H), 3.55 (m, 2H), 3.85 (d, 2H), 3.98 (m, 2H), 4.60 (dd, 1H)

### Preparation example 11: synthesis of 11-(pyridin-4-ylmethyl)tetrahydro-5H,7H-4,9a-(epiminomethano)pyrrolo[2,1-c][1,2,4]dithiazepine-5,10-dione (Compound 11)

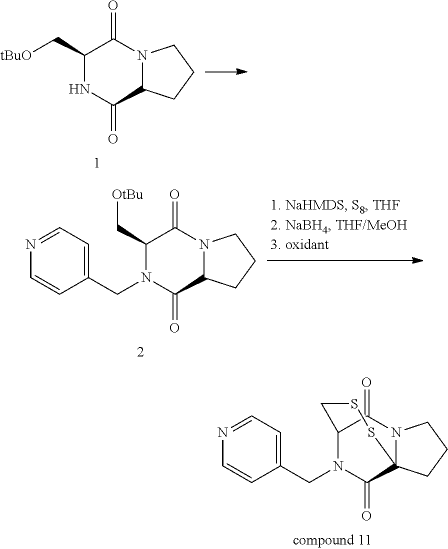

### Step 1. (7S)-7-(tert-butoxymethyl)-8-ethylhexahydropyrazino[2,1-c][1,4]oxazine-6,9-dione (intermediate 2)

It was synthesized from intermediate 1 (the same material as intermediate 4 in preparation example 7) in a similar manner to the method for preparing intermediate 5 of preparation example 7. However, 4-pyridylmethyl chloride was used instead of benzyl bromide.

### Step 2. 11-(pyridin-4-ylmethyl)tetrahydro-5H,7H-4,9a-(epiminomethano)pyrrolo[2,1-c][1,2,4]dithiazepine-5,10-dione (Compound 11)

It was prepared by a similar synthesis method to compound 1 of preparation example 1. That is, using intermediate 2 (0.2 g, 0.60 mmol), sulfur (0.15 g, 8 eq), and NaHMDS (1.2 mL, 2M in THF, 5 eq), 0.6 g of trisulfide intermediate was obtained as a yellow solid. After reduction of trisulfide intermediate (1.0 g, 2.83 mmol) with NaBH4 (0.37 g, 3.5 eq), it was used in the next reaction without purification. Thus, Compound 11 (40 mg) was obtained as a white solid using the reduced intermediate and iodine (0.72 g, 1.0 eq).

¹H NMR (400 MHz, CDCl₃) δ 2.09-2.28 (m, 2H) 2.35 (ddd, J=14.04, 6.60, 1.38 Hz, 1H) 2.56-2.83 (m, 3H) 3.66-3.98 (m, 2H) 4.22 (dd, J=5.44, 2.06 Hz, 1H) 4.41 (d, J=15.64 Hz, 1H) 4.75 (d, J=15.64 Hz, 1H) 7.19 (d, J=5.64 Hz, 2H) 8.61 (br d, J=5.40 Hz, 2H)

LCMS: RT=0.343 min, m/z=321.9 [M+H]⁺

### Preparation example 12: Synthesis of 1,8-dimethyl-6-((6-methylpyridin-2-yl)methyl)-2,3-dithia-6,8-diazabicyclo[3.2.2]nona ne-7,9-dione (Compound 12)

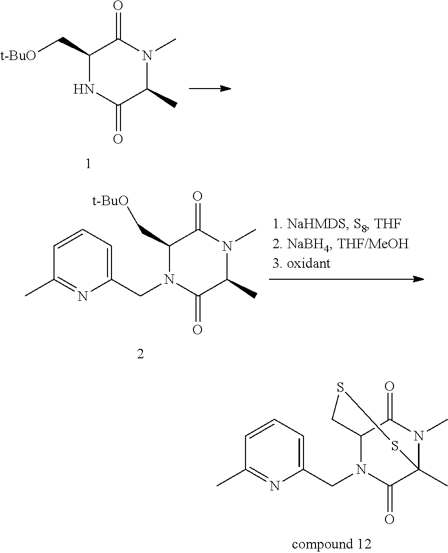

### Step 1. (3S,6S)-3-(tert-butoxymethyl)-1,6-dimethyl-4-((6-methylpyridin-2-yl)methyl)piperazin e-2,5-dione (intermediate 2)

It was synthesized from intermediate 1 (the same material as intermediate 4 in preparation example 1) in a similar manner to the method for preparing intermediate 2 of preparation example 5. However, 6-methyl-2-pyridylmethyl chloride was used instead of quinoline-2-methyl chloride.

### Step 2. 1,8-Dimethyl-6-((6-methylpyridin-2-yl)methyl)-2,3-dithia-6,8-diazabicyclo[3.2.2]nona ne-7,9-dione (Compound 12)

It was prepared by a similar synthesis method to compound 1 of preparation example 1. That is, using intermediate 2 (1.0 g, 3 mmol), sulfur (0.77 g, 8 eq), and NaHMDS (7.5 mL, 2M in THF, 5 eq), 0.47 g of trisulfide intermediate was obtained as a yellow oil. After reduction of trisulfide intermediate (0.47 g, 1.32 mmol) with NaBH₄ (0.15 g, 3.5 eq), it was used in the next reaction without purification. Thus, using the reduced intermediate and iodine (0.67 g, 2.0 eq), 140 mg of compound 12 was obtained as pale-yellow solid.

¹H NMR (400 MHz, CDCl₃) δ 7.56 (t, J=7.60 Hz, 1H), 7.10 (dd, J=16.0, 7.60 Hz, 2H), 4.90 (d, J=14.8 Hz, 1H), 4.58 (dd, J=6.00, 1.60 Hz, 1H), 4.43 (d, J=14.8 Hz, 1H), 3.10 (s, 3H), 2.82-2.90 (m, 1H), 2.68-2.77 (m, 1H), 2.52 (s, 3H), 1.89 ppm (s, 3H).

LCMS: RT=0.376 min, m/z=324.0 [M+H]⁺

### Preparation example 13: Synthesis of 1,8-dimethyl-6-((1-methyl-1H-pyrazol-4-yl)methyl)-2,3-dithia-6,8-diazabicyclo[3.2.2]nonane-7,9-dione (Compound 13)

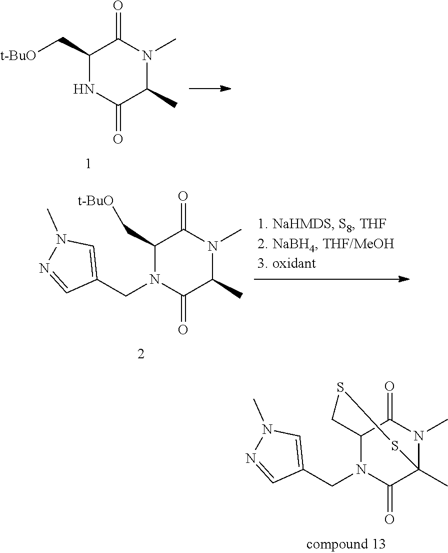 [SMILES: C[C@H]1C(=O)N[C@@H](COC(C)(C)C)C(=O)N1C ; C[C@H]1C(=O)N(Cc2cnn(C)c2)[C@@H](COC(C)(C)C)C(=O)N1C ; CN1C(=O)C2CSSC1(C)C(=O)N2Cc1cnn(C)c1]

### Step 1. (3S,6S)-3-(tert-butoxymethyl)-1,6-dimethyl-4-((1-methyl-1H-pyrazol-4-yl)methyl)pipe razine-2,5-dione (intermediate 2)

It was synthesized from intermediate 1 (the same material as intermediate 4 in preparation example 1) in a similar manner to the method for preparing intermediate 2 of preparation example 5. However, 1-methyl-4-pyrazolemethyl chloride was used instead of quinoline-2-methyl chloride.

### Step 2. 1,8-Dimethyl-6-((1-methyl-1H-pyrazol-4-yl)methyl)-2,3-dithia-6,8-diazabicyclo[3.2.2]nonane-7,9-dione (Compound 13)

It was prepared by a similar synthesis method to compound 1 of preparation example 1. That is, using intermediate 2 (1.0 g, 3.1 mmol), sulfur (0.80 g, 8 eq), and NaHMDS (7.75 mL, 2M in THF, 5 eq), 0.5 g of trisulfide intermediate was obtained as a yellow oil. After reduction of trisulfide intermediate (0.5 g, 1.45 mmol) with NaBH₄ (0.16 g, 3 eq), it was used in the next reaction without purification. Thus, using the reduced intermediate and iodine (1.1 g, 3.0 eq), 201 mg of Compound 13 was obtained as pale white solid.

¹H NMR (400 MHz, CDCl₃) δ 7.35-7.46 (m, 2H), 4.54-4.66 (m, 1H), 4.29-4.44 (m, 2H), 3.90 (d, J=1.60 Hz, 3H), 3.10 (d, J=1.60 Hz, 3H), 2.63-2.76 (m, 2H), 1.89 ppm (d, J=1.60 Hz, 3H).

LCMS: RT=0.409 min, m/z=313.0 [M+H]⁺

### Preparation example 14: Synthesis of 1,8-dimethyl-6-(thiophen-2-ylmethyl)-2,3-dithia-6,8-diazabicyclo[3.2.2]nonane-7,9-di one (Compound 14)

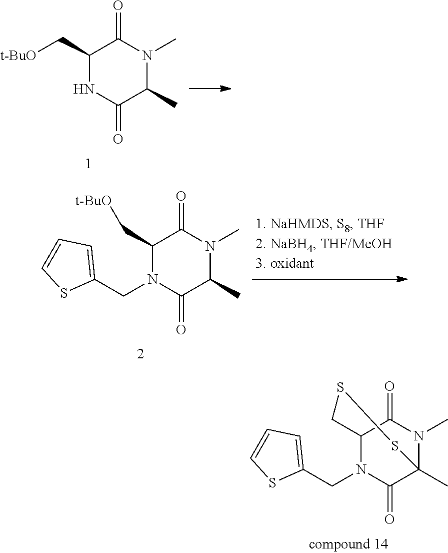 [SMILES: C[C@H]1C(=O)N[C@@H](COC(C)(C)C)C(=O)N1C ; C[C@H]1C(=O)N(Cc2cccs2)[C@@H](COC(C)(C)C)C(=O)N1C ; CN1C(=O)C2CSSC1(C)C(=O)N2Cc1cccs1]

### Step 1. (3S,6S)-3-(tert-butoxymethyl)-1,6-dimethyl-4-(thiophen-2-ylmethyl)piperazine-2,5-dio ne (intermediate 2)

It was synthesized from intermediate 1 (the same material as intermediate 4 in preparation example 1) in a similar manner to the method for preparing intermediate 2 of preparation example 5. However, 2-thiopine methyl chloride was used instead of quinoline-2-methyl chloride.

### Step 2. 1,8-Dimethyl-6-(thiophen-2-ylmethyl)-2,3-dithia-6,8-diazabicyclo[3.2.2]nonane-7,9-di one (compound 14)

It was prepared by a similar synthesis method to compound 1 of preparation example 1. That is, using intermediate 2 (1.0 g, 3.1 mmol), sulfur (0.79 g, 8 eq), and NaHMDS (7.7 mL, 2M in THF, 5 eq), 0.5 g of trisulfide intermediate was obtained as a yellow oil. After reduction of trisulfide intermediate (0.5 g, 1.45 mmol) with NaBH₄ (0.16 g, 3 eq), it was used in the next reaction without purification. Thus, using the reduced intermediate and iodine (1.1 g, 3.0 eq), 201 mg of Compound 14 was obtained as pale white solid.

¹H NMR (400 MHz, CDCl₃) δ 7.29 (d, J=5.20 Hz, 1H), 7.01 (d, J=3.40 Hz, 1H), 6.94-6.99 (m, 1H), 4.79-4.89 (m, 1H), 4.67-4.77 (m, 1H), 4.41 (d, J=6.00 Hz, 1H), 3.09 (s, 3H), 2.64-2.72 (m, 1H), 2.52-2.60 (m, 1H), 1.90 ppm (s, 3H)

LCMS: RT=0.496 min, m/z=315.1 [M+H]⁺

### Preparation example 15: Synthesis of 6-(benzo[d]thiazol-2-ylmethyl)-1,8-dimethyl-2,3-dithia-6,8-diazabicyclo[3.2.2]nonane-7,9-dione (Compound 15)

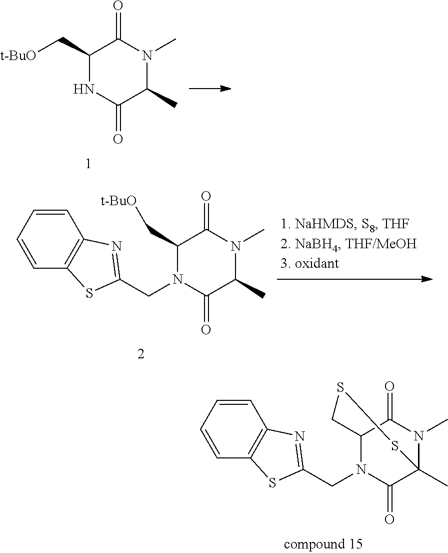 [SMILES: C[C@H]1C(=O)N[C@@H](COC(C)(C)C)C(=O)N1C ; C[C@H]1C(=O)N(Cc2nc3ccccc3s2)[C@@H](COC(C)(C)C)C(=O)N1C ; CN1C(=O)C2CSSC1(C)C(=O)N2Cc1nc2ccccc2s1]

### Step 1. (3S,6S)-1-(benzo[d]thiazol-2-ylmethyl)-6-(tert-butoxymethyl)-3,4-dimethylpiperazine-2,5-dione (intermediate 2)

It was synthesized from intermediate 1 (the same material as intermediate 4 in preparation example 1) in a similar manner to the method for preparing intermediate 2 of preparation example 5. However, 2-benzothiazole methyl chloride was used instead of quinoline-2-methyl chloride.

### Step 2. 6-(Benzo[d]thiazol-2-ylmethyl)-1,8-dimethyl-2,3-dithia-6,8-diazabicyclo[3.2.2]nonane-7,9-dione (Compound 15)

It was prepared by a similar synthesis method to compound 1 of preparation example 1. That is, using intermediate 2 (0.5 g, 1.33 mmol), sulfur (0.34 g, 8 eq), and NaHMDS (3.3 mL, 2M in THF, 5 eq), 0.47 g of trisulfide intermediate was obtained as a brown oil. After reduction of trisulfide intermediate (0.47 g, 1.18 mmol) with NaBH₄ (0.13 g, 3 eq), it was used in the next reaction without purification. Thus, Compound 15 (152 mg) was obtained as yellow solid using the reduced intermediate and iodine (0.6 g, 2.0 eq).

¹H NMR (400 MHz, CDCl₃) δ 8.00 (d, J=8.00 Hz, 1H), 7.88 (d, J=8.00 Hz, 1H), 7.46-7.55 (m, 1H), 7.38-7.46 (m, 1H), 5.15 (d, J=15.6 Hz, 1H), 4.85 (d, J=15.6 Hz, 1H), 4.62 (dd, J=5.60, 2.40 Hz, 1H), 3.12 (s, 3H), 2.71-2.87 (m, 2H), 1.92 ppm (s, 3H).

LCMS: RT=0.409 min, m/z=313.0 [M+H]⁺

### Preparation example 16: Synthesis of 1,6-dimethyl-8-(pyrimidin-2-ylmethyl)-2,3-dithia-6,8-diazabicyclo[3.2.2]nonane-7,9-d ione (Compound 16)

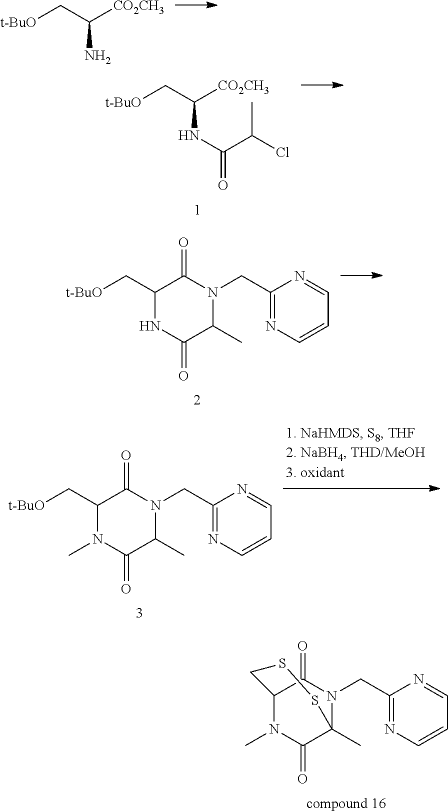 [SMILES: COC(=O)[C@@H](N)COC(C)(C)C ; COC(=O)[C@H](COC(C)(C)C)NC(=O)C(C)Cl ; CC1C(=O)NC(COC(C)(C)C)C(=O)N1Cc1ncccn1 ; CC1C(=O)N(C)C(COC(C)(C)C)C(=O)N1Cc1ncccn1 ; CN1C(=O)C2(C)SSCC1C(=O)N2Cc1ncccn1]

### Step 1. Methyl O-(tert-butyl)-N-(2-chloropropanoyl)-L-serinate (intermediate 1)

2-Chloropropanoyl chloride (26.1 g, 205 mmol) was added to a solution of t-butoxyserine methyl ester (39.5 g, 186 mmol, 1 eq) and TEA (37.8 g, 51.9 mL, 2 eq) in dichloromethane (400 mL). The reaction mixture was stirred at 0° C. for 0.5 h and at 25° C. for 16 h, then diluted with water (100 mL) and extracted with DCM (50 mL). The combined organic layers were washed with brine (100 mL), dried over Na₂SO₄, filtered, and concentrated under reduced pressure to give intermediate 1 (48.6 g, 183 mmol, 97.9% yield) as yellow liquid. It was used in the next step without further purification.

### Step 2. 3-(tert-butoxymethyl)-6-methyl-1-(pyrimidin-2-ylmethyl)piperazine-2,5-dione (intermediate 2)

Pyrimidine-2-ylmethanamine (4.44 g, 40.64 mmol, 1 eq) was added to a mixture of intermediate 1 (10.8 g, 40.6 mmol, 1.00 eq), K₂CO₃ (11.2 g, 81.3 mmol, 2.00 eq) and KI (3.37 g, 20.3 mmol, 0.50 eq) in DMF (120 mL) and stirred for 12 hours. The reaction solution was filtered, and the filtrate was concentrated. The product obtained after purification by prep-HPLC was dissolved in toluene, into which DMAP (433 mg, 0.2 eq) was added, and the mixture was stirred at 130° C. for 36 hours. The reaction mixture was filtered, and the filtrate was concentrated. The residue was purified by prep-HPLC to obtain intermediate 2 (6.04 g, 17.7 mmol, 43.5% yield, 99.0% purity) as yellow oil.

### Step 3. 3-(tert-butoxymethyl)-4,6-dimethyl-1l-(pyrimidin-2-ylmethyl)piperazine-2,5-dione temet(intermediate 3)

NaH (258 mg, 6.46 mmol, 60% pure, 1.10 eq) was added to a solution of intermediate 2 (1.80 m 5.88 mmol. 1.00 ea in DMF (18 mL and the mixture was stirred at 0° C. for 0.5 h, then Mel (834 mg, 5.88 mmol, 365 μL, 1.00 eq) was added to the reaction mixture at 0° C. The reaction was stirred at 25° C. for 2.5 hours and quenched by the addition of MeOH (3 mL), then concentrated under reduced pressure. The residue was purified by prep-HPLC to obtain intermediate 3 as brown oil (1.6 g, 4.99 mmol, 85.0% yield).

### Step 4. 1,6-Dimethyl-8-(pyrimidin-2-ylmethyl)-2,3-dithia-6,8-diazabicyclo[3.2.2]nonane-7,9-d ione (Compound 16)

It was prepared by a similar synthesis method to compound 1 of preparation example 1. That is, using intermediate 3 (1.2 g, 3.75 mmol), sulfur (0.85 g, 8 eq), and NaHMDS (14.2 mL, 2M in THF, 5 eq), 0.64 g of trisulfide intermediate was obtained as a white solid. After reduction of trisulfide intermediate (0.25 g, 0.78 mmol) with NaBH₄ (0.11 g, 4 eq), it was used in the next reaction without purification. Thus, using the reduced intermediate and iodine (0.28 g, 1.5 eq), 110 mg of Compound 16 was obtained as pale white solid.

¹H NMR (400 MHz, CDCl₃) δ 8.70 (d, J=4.88 Hz, 1H) 7.20 (t, J=4.88 Hz, 1H) 5.65 (d, J=17.26 Hz, 1H) 4.38-4.48 (m, 2H) 3.05 (s, 3H) 2.81-2.94 (m, 2H) 1.70 (s, 3H)

LCMS: RT=0.384 min, m/z=311.0 [M+H]⁺

### Preparation example 17: Synthesis of 1,6-dimethyl-8-((1-methyl-1H-imidazol-4-yl)methyl)-2,3-dithia-6,8-diazabicyclo[3.2.2]nonane-7,9-dione (Compound 17)

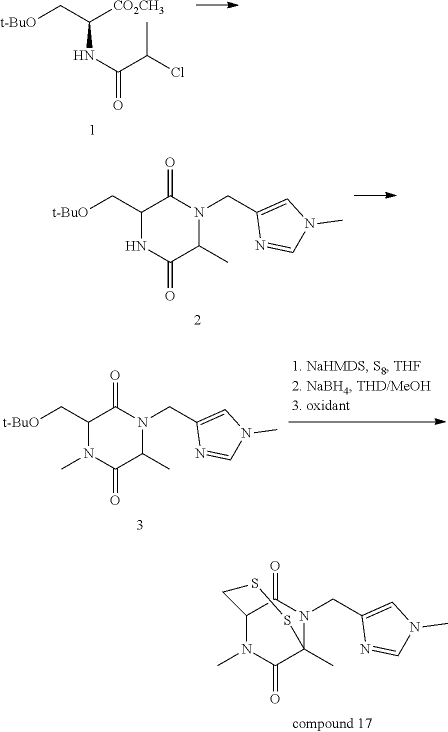

### Step 1. 3-(tert-butoxymethyl)-6-methyl-1-(pyrimidin-2-ylmethyl)piperazine-2,5-dione (intermediate 2)

It was synthesized similarly to the method for preparing intermediate 2 of preparation example 16. However, 1-methylimidazolemethanamine was used instead of pyrimidin-2-ylmethanamine.

### Step 2. 3-(tert-Butoxymethyl)-4,6-dimethyl-1-((1-methyl-1H-imidazol-4-yl)methyl)piperazine-2,5-dione (intermediate 3)

It was synthesized similarly to the method for preparing intermediate 2 of preparation example 16.

### Step 3. 1,6-Dimethyl-8-((1-methyl-1H-imidazol-4-yl)methyl)-2,3-dithia-6,8-diazabicyclo[3.2]nonane-7,9-dione (Compound 17)

It was prepared by a similar synthesis method to compound 1 of preparation example 1. That is, using intermediate 3 (0.5 g, 1.55 mmol), sulfur (0.4 g, 8 eq), and NaHMDS (5.9 mL, 2M in THF, 5 eq), 0.36 g of trisulfide intermediate was obtained as a yellow oil. After reduction of trisulfide intermediate (0.25 g, 0.73 mmol) with NaBH₄ (0.13 g, 5 eq), it was used in the next reaction without purification. Thus, using the reduced intermediate and iodine (0.18 g, 1 eq), 106 mg of Compound 17 was obtained as pale white solid.

¹H NMR (400 MHz, CDCl₃) δ 7.41 (s, 1H) 6.96 (s, 1H) 4.92 (d, J=15.51 Hz, 1H) 4.55 (d, J=15.63 Hz, 1H) 4.31 (t, J=4.00 Hz, 1H) 3.67 (s, 3H) 3.01 (s, 3H) 2.84-2.88 (m, 2H) 2.04 (s, 3H)

LCMS: RT=0.417 min, m/z=313.0 [M+H]⁺

### Preparation example 18: Synthesis of Methyl 4-((1,6-dimethyl-7,9-dioxo-2,3-dithia-6,8-diazabicyclo[3.2.2]nonan-8-yl)methyl)benzo ate (Compound 18)

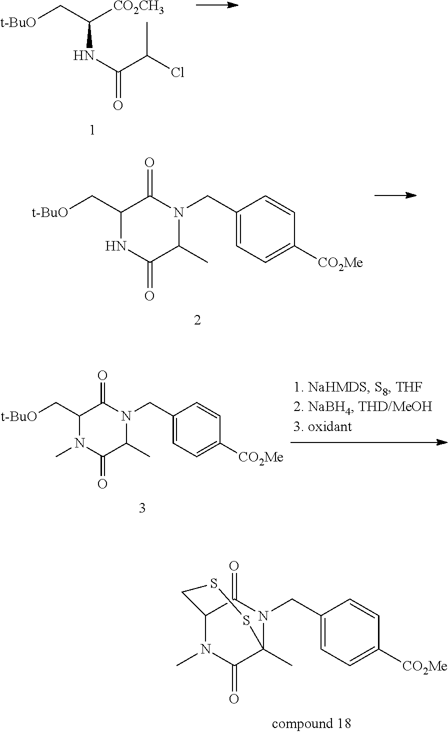 [SMILES: COC(=O)[C@H](COC(C)(C)C)NC(=O)C(C)Cl ; COC(=O)c1ccc(CN2C(=O)C(COC(C)(C)C)NC(=O)C2C)cc1 ; COC(=O)c1ccc(CN2C(=O)C(COC(C)(C)C)N(C)C(=O)C2C)cc1 ; COC(=O)c1ccc(CN2C(=O)C3CSSC2(C)C(=O)N3C)cc1]

### Step 1. Methyl 4-((3-(tert-butoxymethyl)-6-methyl-2,5-dioxopiperazin-1-yl)methyl)benzoate (intermediate 2)

It was synthesized similarly to the method for preparing intermediate 2 of preparation example 16. However, 4-methoxyphenylmethanamine was used instead of pyrimidin-2-ylmethanamine.

### Step 2. Methyl 4-((3-(tert-butoxymethyl)-4,6-dimethyl-2,5-dioxopiperazin-1-yl)methyl)benzoate (intermediate 3)

It was synthesized similarly to the method for preparing intermediate 3 of preparation example 16.

### Step 3. Methyl 4-((1,6-dimethyl-7,9-dioxo-2,3-dithia-6,8-diazabicyclo[3.2.2]nonan-8-yl)methyl)benzo ate (compound 18)

It was prepared by a similar synthesis method to compound 1 of preparation example 1. That is, using intermediate 3 (1.57 g, 4.16 mmol), sulfur (1.1 g, 8 eq), and NaHMDS (15.7 mL, 1M in THF, 5 eq), 1.0 g of trisulfide intermediate was obtained as a yellow oil. After reduction of trisulfide intermediate (0.13 g, 0.32 mmol) with NaBH₄ (0.015 g, 1.2eq), it was used in the next reaction without purification. Thus, Compound 18 (113 mg) was obtained as pale-yellow solid using the reduced intermediate and iodine (0.08 mg, 1 eq).

¹H NMR (400 MHz, CDCl₃) δ 7.99-8.01 (m, 2H) 7.37 (d, J=8.25 Hz, 2H) 5.44 (d, J=16.26 Hz, 1H) 4.43 (dd, J=6.38, 0.88 Hz, 1H) 4.29-4.33 (m, 2H) 3.92 (s, 3H) 3.07 (s, 3H) 2.85 (dd, J=14.88, 6.38 Hz, 1H) 1.70 (s, 3H)

LCMS: RT=0.503 min, m/z=367.1 [M+H]⁺

### Preparation example 19: Synthesis of 1-benzyl-6,8-dimethyl-2,3-dithia-6,8-diazabicyclo[3.2.2]nonane-7,9-dione (Compound 19)

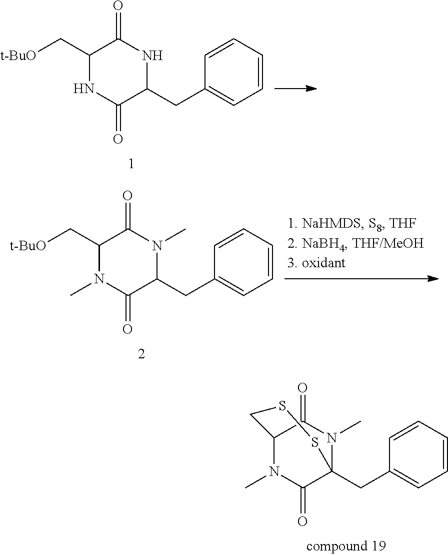 [SMILES: CC(C)(C)OCC1NC(=O)C(Cc2ccccc2)NC1=O ; CN1C(=O)C(Cc2ccccc2)N(C)C(=O)C1COC(C)(C)C ; CN1C(=O)C2(Cc3ccccc3)SSCC1C(=O)N2C]

### Step 1. 3-Benzyl-6-(tert-butoxymethyl)-1,4-dimethylpiperazine-2,5-dione (intermediate 2)

To a solution of compound 1 (15.0 g, 33.6 mmol, 1.00 eq) in DMF (100 mL) was added tBuOK (1M, 134 mL, 4.00 eq) and Mel (29.2 g, 205 mmol, 12.8 mL, 6.12 eq). The mixture was stirred at 25° C. for 2 h, then quenched at 0° C. by adding 300 mL of water and extracted with ethyl acetate (100 mL*2). The combined organic layers were washed with brine (50 mL*6), dried over Na₂SO₄, filtered, and concentrated under reduced pressure to give a residue. The residue was purified by reverse phase HPLC (column: Kromasil Eternity XT 250*80 mm*10 um; mobile phase: [water (NH₄HCO₃)-ACN]; B %: 29%-59%, 20 min) to obtain intermediate 2 (3.20 g, 9.86 mmol, 14.7% yield, 98.1% purity) as white solid.

### Step 2. 1-Benzyl-6,8-dimethyl-2,3-dithia-6,8-diazabicyclo[3.2.2]nonane-7,9-dione (Compound 19)

It was prepared by the same synthetic method to compound 1 of preparation example 1. That is, by using intermediate 2 (1.0 g, 3.14 mmol), sulfur (0.9 g, 8 eq), and NaHMDS (15.6 mL, 1M in THF, 5 eq), 0.5 g of trisulfide intermediate was obtained as a yellow oil. After reduction of trisulfide intermediate (0.5 g, 1.47 mmol) with NaBH₄ (0.3 g, 5 eq), it was used in the next reaction without purification. Thus, Compound 19 (330 mg) was obtained as a white solid using the reduced intermediate and iodine (0.6 g, 1 eq).

¹H NMR (400 MHz, CDCl₃) δ 2.85 (d, J=5.60 Hz, 1H) 2.88-2.96 (m, 1H) 3.02 (s, 3H) 3.10 (s, 3H) 3.24 (d, J=16.4 Hz, 1H) 4.16 (d, J=16.4 Hz, 1H) 4.37 (dd, J=5.6, 2.00 Hz, 1H) 7.14 (d, J=7.2 Hz, 2H) 7.19-7.25 (m, 1H) 7.27-7.32 (m, 2H).

LCMS: RT=0.498 min, m/z=309.0 [M+H]⁺

### Preparation example 20: Synthesis of 6,8-diethyl-1-methyl-2,3-dithia-6,8-diazabicyclo[3.2.2]nonane-7,9-dione (Compound 20)

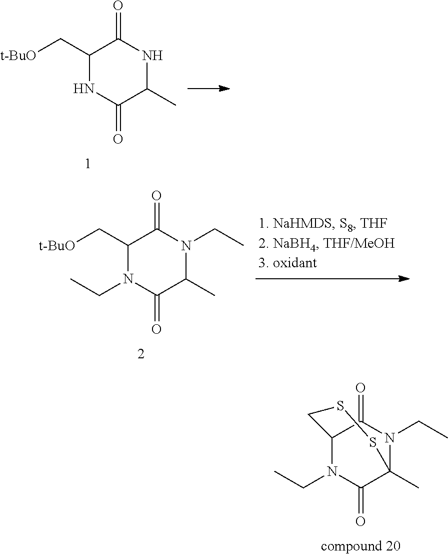 [SMILES: CC1NC(=O)C(COC(C)(C)C)NC1=O ; CCN1C(=O)C(COC(C)(C)C)N(CC)C(=O)C1C ; CCN1C(=O)C2(C)SSCC1C(=O)N2CC]

### Step 1. 3-(tert-Butoxymethyl)-1,4-diethyl-6-methylpiperazine-2,5-dione (intermediate 2)

It was prepared similarly to the synthesis of intermediate 2 in preparation example 19 using starting material 1. However, iodoethane was used instead of iodomethane.

### Step 2. 6,8-Diethyl-1-methyl-2,3-dithia-6,8-diazabicyclo[3.2.2]nonane-7,9-dione (Compound 20)

It was prepared by the same synthetic method as compound 1 of preparation example 1. That is, using intermediate 2 (0.65 g, 2.4 mmol), sulfur (0.8 g, 10 eq), and NaHMDS (13.2 mL, 1M in THF, 5 eq), 0.55 g of trisulfide intermediate was obtained as a yellow oil. After reduction of trisulfide intermediate (0.55 g, 1.47 mmol) with NaBH₄ (0.47 g, 6.6 eq), it was used in the next reaction without purification. Thus, Compound 20 (216 mg) was obtained as a white solid using the reduced intermediate and iodine (0.9 g, 2 eq).

¹H NMR (400 MHz, CDCl₃) δ 4.40 (t, J=3.80 Hz, 1H), 3.68-3.83 (m, 2H), 3.58-3.67 (m, 1H), 3.20 (dd, J=14.0, 7.20 Hz, 1H), 2.80 (d, J=3.60 Hz, 2H), 1.80 (s, 3H), 1.32 (t, J=7.20 Hz, 3H), 1.16 ppm (t, J=7.20 Hz, 3H).

LCMS: RT=0.451 min, m/z=261.0 [M+H]⁺

### Preparation example 21: 12-benzyl-9-methylhexahydro-5H-4,10a-(epiminomethano)pyrazino[2,1-c][1,2,4]dithi azepine-5,11-dione (Compound 21)

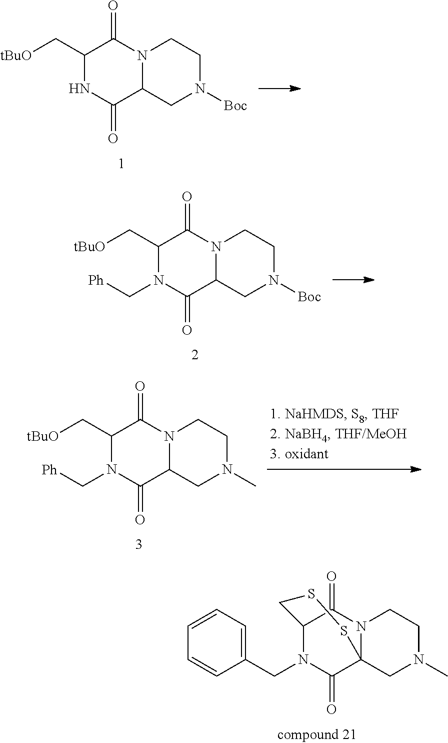 [SMILES: CC(C)(C)OCC1NC(=O)C2CN(C(=O)OC(C)(C)C)CCN2C1=O ; CC(C)(C)OCC1C(=O)N2CCN(C(=O)OC(C)(C)C)CC2C(=O)N1Cc1ccccc1 ; CN1CCN2C(=O)C(COC(C)(C)C)N(Cc3ccccc3)C(=O)C2C1 ; CN1CCN2C(=O)C3CSSC2(C1)C(=O)N3Cc1ccccc1]

### Step 1. tert-Butyl 8-benzyl-7-(tert-butoxymethyl)-6,9-dioxooctahydro-2H-pyrazino[1,2-a]pyrazine-2-car boxylate (intermediate 2)

To a solution of compound 1 (20.0 g, 56.3 mmol, 1.00 eq) in THF (500 mL) was added NaH (6.75 g, 169 mmol, 60% pure, 3.00 eq) at 0° C. over 30 min. Benzyl bromide (10.6 g, 61.9 mmol, 7.35 mL, 1.10 eq) was added dropwise at 0° C. and stirred at 25° C. for 12 hours. The reaction mixture was quenched at 0° C. by adding 100 mL of water and extracted with ethyl acetate (100 mL*2). The combined organic layers were washed with brine (100 mL), dried over Na₂SO₄, filtered, and concentrated under reduced pressure to give a residue. The residue was purified by column chromatography (SiO₂, petroleum ether:ethyl acetate=2:1 to 1:3) to give intermediate 2 (15.0 g, 17.2 mmol, 76.5% purity) as white solid.

### Step 2. 8-Benzyl-7-(tert-butoxymethyl)-2-methylhexahydro-2H-pyrazino[1,2-a]pyrazine-6,9-d ione (intermediate 3)

To a solution of intermediate 2 (10.0 g, 22.4 mmol, 1.00 eq) in EtOAc (70.0 mL) was added HCV/EtOAc (4M, 30.0 mL, 5.35 eq) and stirred at 25° C. for 2 hours. The reaction mixture was concentrated under reduced pressure and the residue was used in the next step without further purification. After dissolving this compound in MeOH (150 mL), formaldehyde (17.6 g, 217 mmol, 16.2 mL, 37.0% purity, 5.00 eq) was added at 25° C. for 30 minutes, followed by NaBH(OAc)₃ (13.8 g, 65.1 mmol, 1.50 eq) addition. The resulting mixture was stirred at 25° C. for 1 hour and the reaction mixture was concentrated under reduced pressure to give a residue. Intermediate 3 (5.50 g, 15.2 mmol, 34.9% yield, 100% purity) was obtained after purification by reverse phase HPLC (column: Kromasil Eternity XT 250*80 mm*Oum; mobile phase: [water (NH₄HCO₃)-ACN]; B %: 23%-53%, 20 min) as yellow oil. Two isomers (diastereomers) were formed, and two were isolated from each other.

Isomer 1: ¹H NMR (400 MHz, DMSO-_{d6}) δ ppm 1.05 (s, 9H) 1.76-1.90 (m, 1H) 2.17-2.37 (m, 4H) 2.65-2.80 (m, 2H) 3.06-3.20 (m, 2H) 3.58 (s, 2H) 3.94-4.15 (m, 2H) 4.24-4.39 (m, 2H) 4.84 (d, J=15.2 Hz, 1H) 7.10-7.45 (m, 5H).

Isomer 2: ¹H NMR (400 MHz, DMSO-_{d6}) δ 0.99-1.10 (m, 9H) 1.73-1.92 (m, 1H) 2.04-2.33 (m, 4H) 2.61-2.82 (m, 2H) 3.09 (dd, J=10.4, 2.69 Hz, 1H) 3.58 (d, J=2.00 Hz, 2H) 3.88-4.03 (m, 1H) 4.09-4.17 (m, 1H) 4.18-4.39 (m, 2H) 4.85 (d, J=15.2 Hz, 1H) 7.24-7.35 (m, 5H)

MS: m/z=360.2

### Step 3. 12-Benzyl-9-methylhexahydro-5H-4,10a-(epiminomethano)pyrazino[2,1-c][1,2,4]dithi azepine-5,11-dione (Compound 21)

It was prepared by a similar synthesis method to compound 1 of preparation example 1. That is, 0.48 g of the trisulfide intermediate was obtained as a yellow solid using intermediate 3 (isomer 1 or 2, 1.2 g, 3.3 mmol), sulfur (0.86 g, 8 eq), and NaHMDS (16.7 mL, 1 M in THF, 5 eq). After reduction of trisulfide intermediate (0.78 g, 1.47 mmol) with NaBH₄ (0.14 g, 1.9 eq), it was used in the next reaction without purification. Thus, using the reduced intermediate and iodine (0.68 g, 1 eq), 101 mg of Compound 21 was obtained as white solid.

¹H NMR (400 MHz, CDCl₃) δ 2.14 (td, J=12.0, 3.63 Hz, 1H) 2.43 (s, 3H) 2.56-2.80 (m, 3H) 2.87-3.03 (m, 1H) 3.16-3.37 (m, 2H) 4.13-4.29 (m, 2H) 4.44 (d, J=14.8 Hz, 1H) 4.82 (d, J=14.8 Hz, 1H) 7.26-7.31 (m, 3H) 7.35-7.39 (m, 2H).

LCMS: RT=0.520 min, m/z=350.1 [M+H]⁺

### Preparation example 22: Synthesis of 12-benzyl-9-methylhexahydro-5H-4,10a-(epiminomethano)pyrazino[2,1-c][1,2,4]dithi azepine-5,11-dione (Compound 22)

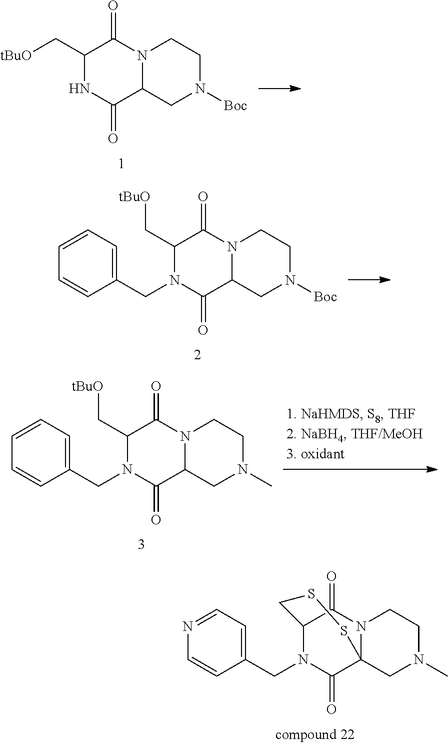 [SMILES: CC(C)(C)OCC1NC(=O)C2CN(C(=O)OC(C)(C)C)CCN2C1=O ; CC(C)(C)OCC1C(=O)N2CCN(C(=O)OC(C)(C)C)CC2C(=O)N1Cc1ccccc1 ; CN1CCN2C(=O)C(COC(C)(C)C)N(Cc3ccccc3)C(=O)C2C1 ; CN1CCN2C(=O)C3CSSC2(C1)C(=O)N3Cc1ccncc1]

### Step 1. tert-Butyl 7-(tert-butoxymethyl)-6,9-dioxo-8-(pyridin-4-ylmethyl)octahydro-2H-pyrazino[1,2-a]p yrazin-2-carboxylate (intermediate 2)

It was prepared similarly to the synthesis of intermediate 2 in preparation example 21. However, 4-pyridylmethyl bromide was used instead of benzyl bromide.

### Step 2. 7-(tert-butoxymethyl)-2-methyl-8-(pyridin-4-ylmethyl)hexahydro-2H-pyrazino[1,2-a]p yrazine-6,9-dione (intermediate 3)

It was prepared similarly to the synthesis of intermediate 3 in preparation example 21. Two isomers (diastereomers) were formed, and each isomer was isolated.

Isomer 1: ¹H NMR (400 MHz, DMSO-_{d6}) δ 1.05 (d, J=2.80 Hz, 9H) 1.75-1.98 (m, 2H) 2.19-2.37 (m, 3H) 2.59-2.90 (m, 2H) 3.03-3.29 (m, 3H) 3.44-3.66 (m, 2H) 3.97-4.14 (m, 2H) 4.17-4.59 (m, 2H) 4.60-4.95 (m, 1H) 7.28 (dd, J=18.4, 5.76 Hz, 2H) 8.51 (t, J=6.00 Hz, 2H).

Isomer 2: ¹H NMR (400 MHz, DMSO-_{d6}) δ 1.04 (d, J=2.80 Hz, 9H) 1.75-2.00 (m, 2H) 2.25 (d, J=9.20 Hz, 3H) 2.75 (br s, 2H) 3.03-3.39 (m, 2H) 3.47 (br dd, J=9.20, 2.38 Hz, 1H) 3.60 (d, J=2.00 Hz, 1H) 3.98-4.07 (m, 1H) 4.09-4.17 (m, 1H) 4.18-4.57 (m, 2H) 4.59-4.92 (m, 1H) 7.28 (dd, J=18.0, 5.88 Hz, 2H) 8.37-8.60 (m, 2H)

### Step 3. 12-Benzyl-9-methylhexahydro-5H-4,10a-(epiminomethano)pyrazino[2,1-c][1,2,4]dithi azepine-5,11-dione (Compound 22)

It was prepared by a similar synthesis method to compound 1 of preparation example 1. That is, 0.40 g of the trisulfide intermediate was obtained as a yellow solid using intermediate 3 (isomer 1 or 2, 1.2 g, 3.3 mmol), sulfur (0.85 g, 8 eq), and NaHMDS (16.6 mL, 1 M in THF, 5 eq). After reduction of trisulfide intermediate (0.55 g, 1.44 mmol) with NaBH₄ (0.18 g, 3.2 eq), it was used in the next reaction without purification. Thus, using the reduced intermediate and iodine (0.53 g, 1 eq), Compound 22 (102 mg) was obtained as yellow solid.

¹H NMR (400 MHz, CDCl₃) δ 2.16 (br d, J=3.60 Hz, 1H) 2.43 (s, 3H) 2.62 (d, J=13.6 Hz, 1H) 2.69-2.86 (m, 2H) 2.89-3.04 (m, 1H) 3.16-3.35 (m, 2H) 4.14-4.38 (m, 3H) 4.92 (d, J=15.6 Hz, 1H) 7.19 (d, J=5.60 Hz, 2H) 8.61 (d, J=5.60 Hz, 2H).

LCMS: RT=0.531 min, m/z=351.3 [M+H]⁺

### Preparation example 23: 12-benzyl-9-methylhexahydro-5H-4,10a-(epiminomethano)pyrazino[2,1-c][1,2,4]dithi azepine-5,11-dione (Compound 23)

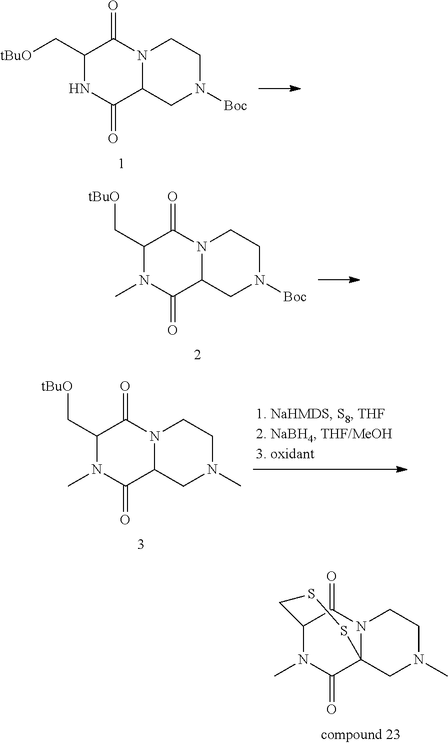

### Step 1. tert-Butyl 7-(tert-butoxymethyl)-8-methyl-6,9-dioxooctahydro-2H-pyrazino[1,2-a]pyrazine-2-car boxylate (intermediate 2)

It was prepared similarly to the synthesis of intermediate 2 in preparation example 21. However, methyl iodide was used instead of benzyl bromide.

### Step 2. 7-(tert-butoxymethyl)-2,8-dimethylhexahydro-2H-pyrazino[1,2-a]pyrazine-6,9-dione (intermediate 3)

It was prepared similarly to the synthesis of intermediate 3 in preparation example 21.

### Step 3. 12-Benzyl-9-methylhexahydro-5H-4,10a-(epiminomethano)pyrazino[2,1-c][1,2,4]dithi azepine-5,11-dione (Compound 23)

It was prepared by a similar synthesis method to compound 1 of preparation example 1. That is, using intermediate 3 (4.0 g, 14.1 mmol), sulfur (3.6 g, 8 eq), and NaHMDS (70.6 mL, 1M in THF, 5 eq), 2.1 g of trisulfide intermediate was obtained as a white solid. After reduction of trisulfide intermediate (0.50 g, 1.44 mmol) with NaBH₄ (0.26 g, 5 eq), it was used in the next reaction without purification. Thus, using the reduced intermediate and DDQ (0.33 g, 0.9 eq), Compound 23 (125 mg) was obtained as pale yellow solid.

¹H NMR (400 MHz, CDCl₃) δ 4.24 (dd, J=4.9, 3.1 Hz, 1H), 4.19 (dd, J=13.4, 2.0 Hz, 1H), 3.30-3.22 (m, 2H), 3.03 (s, 3H), 2.97-2.87 (m, 3H), 2.53 (d, J=13.6 Hz, 1H), 2.40 (s, 3H), 2.10 (td, J=12.2, 3.6 Hz, 1H).

¹³C NMR (126 MHz, CDCl3) δ 169.49, 166.15, 68.29, 62.86, 59.28, 52.72, 46.14, 40.01, 33.07, 29.37.

MS: m/z=274.0715 [M+H]⁺

### Preparation example 24: Synthesis of 6-benzyl-1,4,8-trimethyl-2,3-dithia-6,8-diazabicyclo[3.2.2]nonane-7,9-dione (Compound 24)

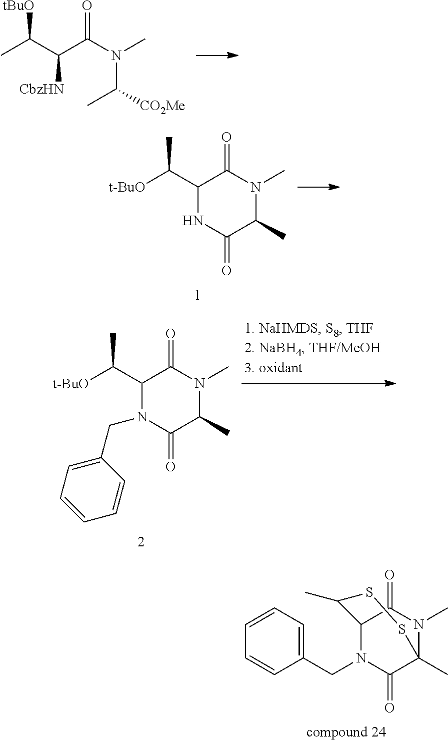

### Step 1. (6S)-3-((R)-1-(tert-butoxy)ethyl)-1,6-dimethylpiperazine-2,5-dione (intermediate 1)

It was similarly prepared to the synthesis of intermediate 4 in preparation example 7.

### Step 2. (3S)-1-Benzyl-6-((S)-1-(tert-butoxy)ethyl)-3,4-dimethylpiperazine-2,5-dione (intermediate 2)

It was prepared in a similar manner to the synthesis of intermediate 2 in preparation example 2.

### Step 3. 6-Benzyl-1,4,8-trimethyl-2,3-dithia-6,8-diazabicyclo[3.2.2]nonane-7,9-dione (Compound 24)

It was prepared by a similar synthesis method to compound 1 of preparation example 1. That is, using intermediate 2 (0.9 g, 2.71 mmol), sulfur (0.7 g, 8 eq), and NaHMDS (10.9 mL, 1M in THF, 5 eq), the trisulfide intermediate was obtained as a yellow solid. After reduction of trisulfide intermediate (0.49 g, 1.38 mmol) with NaBH₄ (0.16 g, 3 eq), it was used in the next reaction without purification. Thus, Compound 24 was obtained using the reduced intermediate and iodine (0.7 g, 2 eq), where two isomers (diastereomers) were formed, with isomer 1 being yellow solid (157 mg) and isomer 2 being white solid (13 mg).

Isomer 1: ¹H NMR (400 MHz, CDCl₃) δ δ 7.22-7.33 (m, 3H), 7.15-7.22 (m, 2H), 4.41-4.68 (m, 2H), 3.88 (s, 1H), 3.02 (s, 3H), 2.67 (q, J=7.20 Hz, 1H), 1.83 (s, 3H), 1.11 (d, J=7.20 Hz, 3H)

Isomer 2: ¹H NMR (400 MHz, CDCl₃) δ 7.28-7.38 (m, 3H), 7.10-7.18 (m, 2H), 5.45 (d, J=15.2 Hz, 1H), 4.19 (d, J=6.00 Hz, 1H), 3.84 (d, J=15.2 Hz, 1H), 3.08 (s, 3H), 2.96-3.04 (m, 1H), 1.91 (s, 3H), 1.57-1.60 (m, 3H)

LCMS (isomer 1): RT=1.925 min, m/z=323 [M+H]⁺

LCMS (isomer 2): RT=1.894 min, m/z=323 [M+H]⁺

### Preparation example 25: Synthesis of 6-benzyl-1,4,8-trimethyl-2,3-dithia-6,8-diazabicyclo[3.2.2]nonane-7,9-dione (Compound 25)

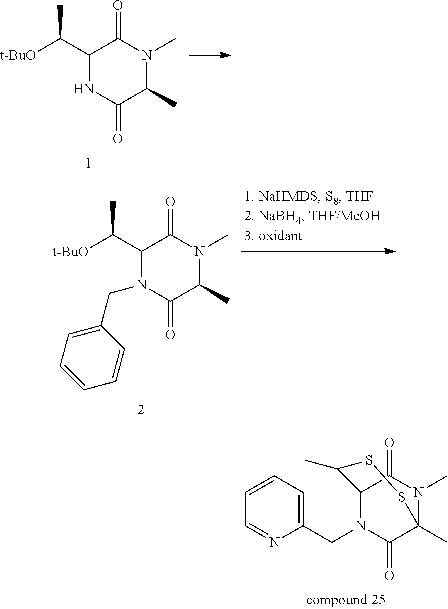 [SMILES: C[C@H](OC(C)(C)C)C1NC(=O)[C@H](C)N(C)C1=O ; C[C@H](OC(C)(C)C)C1C(=O)N(C)[C@@H](C)C(=O)N1Cc1ccccc1 ; CC1SSC2(C)C(=O)N(Cc3ccccn3)C1C(=O)N2C]

### Step 1. (6S)-3-((S)-1-(tert-butoxy)ethyl)-1,6-dimethyl-4-(pyridin-2-ylmethyl)piperazine-2,5-di one (intermediate 2)

It was prepared in a similar manner to the synthesis of intermediate 2 in preparation example 2. However, 2-pyridylmethyl bromide was used instead of benzyl bromide.

### Step 2. 6-Benzyl-1,4,8-trimethyl-2,3-dithia-6,8-diazabicyclo[3.2.2]nonane-7,9-dione (Compound 25)

It was prepared by a similar synthesis method to compound 1 of preparation example 1. That is, by using intermediate 2 (3.1 g, 9.3 mmol), sulfur (2.4 g, 8 eq), and NaHMDS (37 mL, 1M in THF, 5 eq), 1.67 g of trisulfide intermediate was obtained as a yellow oil. After reduction of trisulfide intermediate (1.67 g, 4.7 mmol) with NaBH₄ (0.53 g, 3 eq), it was used in the next reaction without purification. Thus, using the reduced intermediate and iodine (1 g, 1 eq), Compound 25 (373 mg) was obtained as white solid.

¹H NMR (400 MHz, CDCl₃) δ 8.52 (d, J=4.60 Hz, 1H) 7.62 (td, J=7.60, 1.60 Hz, 1H) 7.12-7.23 (m, 2H) 5.44 (d, J=15.6 Hz, 1H) 4.49 (d, J=15.6 Hz, 1H) 3.95-4.09 (m, 3H) 2.96 (s, 3H) 1.68 (d, J=7.20 Hz, 3H) 1.33 (d, J=6.00 Hz, 3H) 1.21 (s, 9H)

LCMS: RT=0.684 min, m/z=323.9 [M+H]⁺

### Experimental Example 1: Materials

A rabbit polyclonal antibody specific to peroxiredoxin type 2 (Prx II or Prdx2) was purchased from AbFrontier (Seoul, Korea). The sequences of siRNA specific to human Prx II used in the present invention is as follows:

5′-CGCUUGUCUGAGGAUUACGUU-3′
(Prx II-1)
5′-AGGAAUAUUUCUCCAAACAUU-3′
(Prx II-2).

Phospho-Tyrosine antibody (4G10) and PDGF-BB were purchased from Upstate. Prx II, Prx-SO_{2/3}, and phospho-PDGFR-β(pY857) rabbit polyclonal antibodies were prepared as described in literature (M. H. Choi et al., Nature 2005 May 19; 435(7040):347-53). Phospho-VEGFR2 (pY1175), VEGFR2, phospho-PLCη1 (pY783), PLCη1, pTpY-Erk, Erk2 antibodies were purchased from Cell Signaling Technology (CST). Alpha-Tubulin antibody was purchased from Sigma.VEGF-A was purchased from R&D.

### Experimental Example 2: Cell Culture

Human aortic endothelial cells (HAEC), human pulmonary artery endothelial cells (PAEC), human aortic smooth muscle cells (HASMC), human pulmonary artery smooth muscle cells (PASMC) were purchased from BioWhittaker (Belgium). They were seeded on a 0.1% gelatin-coated culture plate. They were grown in Endothelial basal medium (EBM-2) and Smooth Muscle Cell Basal Medium (SmBM) containing 10% fetal bovine serum (FBS) and full medium supplement (Clonetics-BioWhittaker Cat. no. cc-4176 for HAEC and PAEC; Clonetics-BioWhittaker Cat. no. cc-4149 for HASMC and PASMC). Cell culture was carried out in a humidified CO₂ incubator under 5% carbon dioxide at 37° C. Cells at 5 to 7 passages were mainly used in the invention.

### Experimental Example 3: Peroxidase Activity Assay

Hydrogen peroxide (H₂O₂)-reducing peroxidase activity of compounds in present invention was performed as follows. Peroxyredoxin activity was measured in reaction mixture (200 μL volume) containing 50 mM HEPES buffer (pH 7.0), 1 mM EDTA, 250 μM NADPH, 3 μM yeast thioredoxin (Trx), 1.5 μM yeast Trx reductase (TR), 50 μM test compound, and 1.2 mM H₂O₂.

Reaction was initiated by adding H₂O₂ and the decrease in absorbance at 340 nm was measured using Agilent UV8453 spectrophotometry (Hewlett Packard, USA) at 30° C. for 12 min. Initial reaction rate is the amount of NADPH oxidized per minute calculated from slope of the linear part of curve.

### Experimental Example 4: Measurement of Intracellular H₂O₂-Eliminating Activity

The activity of test compounds in present invention to eliminate intracellular H₂O₂ was measured as follows. NIH-3T3 cells (ATCC) were seeded in a 35-mm culture dish at 2×10⁵ cells/well. After culture for 24 hours, cells were serum-starved with phenol red free-basal DMEM containing 0.5% FBS for 6 h. Starved NIH 3T3 cells were pretreated with compounds diluted with phenol red free-basal DMEM at concentrations (0, 12.5, 25, 50, 100 mM) for 30 min, followed by treatment with 20 mU of glucose oxidase (Gox) for 30 min. A reactive oxygen species-sensitive fluorescence dye, carboxymethyl-dichlorofluorescein-diacetate (CM-H₂DCF-DA), which was prepared in 1 mM DMSO stock and diluted 500 times in phenol red free-basal DMEM, was added to the cells and incubated for 5 min. Then, cells were quickly rinsed with basal medium. Images were taken at excitation 497 nm/emission 518 nm settings using a fluorescence microscope. Three images at 10× magnification per sample were obtained, and fluorescence intensities from 30 or more individual cells per image were averaged.

### Experimental Example 5: Cytotoxicity Assay

For cytotoxicity analysis, human aortic endothelial cells (HAEC) and smooth muscle cells (HASMC) were seeded in a 96-well culture plate at density of 4000 cells/well in a final volume of 100 μL. Cells were cultured for 24 h and serum-starved for an additional 18 h. Test compounds were serially diluted with EBM-2 culture medium, added to the plate at 100 μL/well, and further incubated for 24 h. The number of viable cells was measured using the WST-1 cell viability assay kit (Roche Diagnostics, USA). Cell number was expressed as absorbance values at 450 nm after subtracting the turbidity value at 600 nm. The absorbance values from three replicate wells were averaged.

### Experimental Example 6: Immunoblot Analysis

Cells were quickly washed in ice-cold phosphate-buffered saline (PBS) and lysed in extraction buffer containing 20 mM Hepes (pH 7.0), 1% Triton X-100, 150 mM NaCl, 10% glycerol, 1 mM EDTA, 2 mM EGTA, and 1 mM DTT, 5 mM Na₃VO₄, 5 mM NaF, 1 mM AEBSF, aprotinin (5 μg/mL), and leupeptin (5 μg/mL). After cen-trifugation at 12,000×g, the clarified cell extracts were used for immunoblotting. For re-blotting, the immunoblots were retrieved in a solution of 67 mM Tris (pH 6.7), 2% SDS, and 100 mM 2-mercaptoethanol at 60° C. for 30 min to remove antibodies and rinsed three times with with a Tris-buffered saline (TBS) solution containing 1% Triton X-100. The retrieved membranes were subjected to new immunoblotting.

### Experimental Example 7: Measurement of Redox Potential

As shown in Table 1 below, 10 kinds of redox buffer solutions (0.5 mL) are made by mixing with 50 mM solutions of reduced dithiothreitol (DTT) and its oxidized DTT (trans-1,2-dithiane-4,5-diol) at indicated ratios just before use. A test compound (10 mM) was diluted 20-fold with each redox buffer solution and incubated for 3 h in a 30° C. water bath. Then, compounds were analyzed by HPLC. The area of peaks corresponding to the oxidized and reduced forms of test compound based on specific retention time was calculated and plotted as percent of total peak area against the Nernst equation value (mV) at x-axis. The Nernst equation value corresponding to 50% reduction of test compound was set as the midpoint redox potential value for test compound.

TABLE 1

| Ratio | DTT(Red) | DTT(Ox) | Nernst |
|---|---|---|---|
| DTT(Red)/DTT(Ox) | (μL) | (μL) | equation(mV) |
| 4 | 400 | 100 | −341 |
| 1 | 250 | 250 | −323 |
| 0.25 | 100 | 400 | −305 |
| 0.11 | 50 | 450 | −294 |
| 0.05 | 25 | 475 | −285 |
| 0.02 | 10 | 490 | −272 |
| 0.01 | 5 | 495 | −263 |
| 0.005 | 2.5 | 497.5 | −254 |
| 0.002 | 1 | 499 | −242 |
| 0.0004 | 0.2 | 499.8 | −221 |

Specific methods and conditions other than Experimental Examples 1-7 were performed similarly to those disclosed in International Patent Application Publication WO2013-077709 or WO2018-008984. All contents disclosed in International Patent Application Publications WO2013-077709 and WO2018-008984 are hereby incorporated by reference.

### Experimental Example 8: Growth Factor-Induced Proliferation Assay in Vascular Smooth Muscle Cell and Vascular Endothelial Cell

The ability of test compounds to control cell proliferation was measured in vascular smooth muscle cells and vascular endothelial cells.

HASMCs and PASMCs were transfected with a PrxII-specific siRNA using RNAi MAX transfection reagent according to manufacturer's protocol. After 24 hours, transfected cells (3,000 cells/well) were seeded in a 96-well culture plate. At 12 h after seeding, cells were serum-starved for additional 18 h in SmBM basal medium containing 0.5% FBS. Test compounds were serially diluted with the same basal medium containing 0.5% FBS, added to the cells (100 mL/well), and incubated for 2 h. After treatment, cells were placed in fresh SmBM medium containing 0.5% FBS and PDGF-BB (25 ng/mL) for growth factor stimulation and further cultured for 24 h.

HAECs and PAECs were transfected with a PrxII-specific siRNA using RNAi MAX transfection reagent according to manufacturer's protocol. After 24 hours, transfected cells (3,000 cells/well) were seeded in a 96-well culture plate. At 12 h after seeding, cells were serum-starved for additional 18 h in EBM basal medium containing 0.5% FBS. Test compounds were serially diluted with the same basal medium containing 0.5% FBS, added to the cells (100 mL/well), and incubated for 2 h. After treatment, cells were placed in fresh EBM medium containing 0.5% FBS and VEGF-A (25 ng/mL) for growth factor stimulation and further cultured for 24 h.

The degree of cell proliferation was measured using the Cell Titer-GLO kit (Roche Diagnostics, USA). Data are the percent of luminescence intensity averaged from three replicate wells versus that of untreated control group.

### Experimental Example 9: Efficacy Evaluation in Preclinical Rat Model of Pulmonary Arterial Hypertension

The rat study protocol was approved by the Institutional Animal Care and Use Committee of the Ewha Womans University, Republic of Korea, and conforms to the ARRIVE guidelines. Six-week-old rats were acclimatized for one week in the laboratory. Then, Sugen 5416 (Sigma Aldrich) was subcutaneously injected at a dose of 20 mg/kg. The administered subjects were maintained in a normobaric hypoxic (10% O₂) chamber (A chamber, Biosphenix) for 3 weeks. Rats were transferred to normoxia and orally administered with either control vehicle or test compounds for additional 5 weeks (P.O., once daily, 0.1 mg per kg weight).

Right ventricular systolic pressure (RVSP) was measured by echocardiography using a curved catheter equipped with PowerLab2/26 device (AD Instruments, UK). Rats were anesthetized with 2% isoflurane inhalation. A catheter was inserted through the right jugular vein, the pressure was measured for 10 seconds with stabilized pattern. After echocardiography, rats were perfused with saline, and the heart was carefully removed. The removed heart was dissected into the right ventricle (RV), the rest of the septum (S; septum), and the left ventricle (LV). Each piece of heart was weighed using a microbalance. The RV/(LV+S) value was calculated and represented as a right ventricular hypertrophy index (Fulton's index).

### Experimental Example 10: Histological Analysis

Rats were anesthetized with 2% isoflurane inhalation and subjected to transcardiac perfusion-fixation with heparinized saline solution containing 37% formaldehyde. Then, left lung lobes were incised and fixed in 10% NBF for 3 days. The fixed tissues were paraffin-embedded and sectioned using a rotary microtome (Leica HistoCore MULTICUT). Two serial tissue sections (4 μm thick) were placed a slide glass and stained with hematoxylin and eosin (HE). For analysis, ˜100 pulmonary arteries (diameter 20-100 μm) per tissue section were chosen for measurement. The luminal, internal elastic laminal, and external elastic laminal areas were quantified using NIH ImageJ v1.62. The intimal and medial areas were determined by subtraction of the luminal area from the internal elastic area and by subtracting the internal elastic area from the external elastic area. The values were averaged and used for calculating pulmonary arterial vessel thickness.

### Experimental Example 11: Immunofluorescence Staining

Paraffin sections were blocked with 5% normal donkey serum (Vector Laboratories) in PBST (PBS solution of 0.3% Triton X-100) for 1 h at room temperature. Thereafter, lung tissue sections were incubated with antigens for Alexa Fluor 568-conjugated smooth muscle actin (α-SMA; 1:300 dilution) and Alexa Fluor 488-conjugated von Willebrand Factor (vWF; 1:200 dilution) at 4° C. for 12 h. Subsequently, nuclei were counterstained with DAPI. Fluorescence images were recorded in random regions of at least 10 pulmonary artery vessels (diameter 20-100 um) per tissue section at a screen magnification of 60× using an LSM 880 confocal microscope equipped with argon and helium-neon lasers.

Result 1: Peroxidase Activity for H₂O₂ Reduction

The H₂O₂-reducing peroxidase activity of the test compounds in present invention was measured in a reaction mixture containing thioredoxin (Trx)/thioredoxin reductase (TR), respectively. Data are summarized in the table below.

TABLE 2

| Compound no. | Peroxidase activity for H₂O₂ reduction(nmole/min) |
|---|---|
| Compound 1 | 4.92 |
| Compound 2 | 5.05 |
| Compound 3 | 4.11 |
| Compound 4 | 3.82 |
| Compound 5 | 4.80 |
| Compound 6 | 4.57 |
| Compound 7 | 3.83 |
| Compound 8 | 4.91 |
| Compound 9 | 5.19 |
| Compound 10 | 5.06 |
| Compound 11 | 4.57 |
| Compound 12 | 4.94 |
| Compound 13 | 4.78 |
| Compound 14 | 4.78 |
| Compound 15 | 4.79 |
| Compound 16 | 4.93 |
| Compound 17 | 4.80 |
| Compound 18 | 4.91 |
| Compound 19 | 5.69 |
| Compound 20 | 4.78 |
| Compound 21 | 8.38 |
| Compound 22 | 7.97 |
| Compound 23 | 8.81 |
| Compound 24 | 4.26 |
| Compound 25 | 4.51 |
| Control compound (A5) | 4.25 |

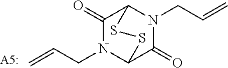 [SMILES: C=CCN1C(=O)C2SSC1C(=O)N2CC=C]

As shown in Table 2, most of the test compounds exhibit excellent H₂O₂-reducing peroxidase activity in the presence of the Trx/TR redox system. This indicates that the new compounds have peroxidase activity that specifically mimics 2-Cys peroxiredoxin.

Compared to a control compound belonging to epidithiodioxopiperazine class (A5), new compounds in the present invention have similar or better peroxidase activity.

Result 2: Assay of Intracellular H₂O₂-Eliminating Activity

The intracellular H₂O₂-eliminating activity of test compounds in present invention was measured using a live-cell imaging fluorescence probe (CM-HaDCF-DA). H₂O₂-eliminating activity was measured depending on ascending concentrations of the test compound and expressed as the percent of activity compared to the untreated control group. EC₅₀ represents the concentration of test compound at 50% reduction of intracellular H₂O₂.

TABLE 3

| Compound | EC₅₀ (nM) |
|---|---|
| 1 | 8.25 |
| 2 | 6.47 |
| 5 | 7.77 |
| 6 | 5.99 |
| 8 | 6.57 |
| A5 | 15.21 |

As shown in Table 3, test compounds show excellent H₂O₂-eliminating ability compared to a control compound belonging to epidithiodioxopiperazine class (A5).

### Result 3: Measurement of Oxidation-Reduction Potential

The redox potential values of the test compounds were analyzed based on the potential values established in redox buffer solutions where reduced and oxidized DTT are mixed at various ratios. Data are shown in the table below.

TABLE 4

| | Midpoint redox |
|---|---|
| Compound | potential (E m; mV) |
| Compound 1 | −285.25 |
| Compound 2 | −293.19 |
| Compound 3 | −296 |
| Compound 4 | −292.74 |
| Compound 5 | −290.3 |
| Compound 6 | −290.39 |
| Compound 7 | −300.98 |
| Compound 8 | −272.6 |
| Compound 10 | −268.53 |
| Compound 20 | −296.79 |
| Compound 21 | −272.86 |
| Trx | −270 |
| GSH | −240 |

As shown in Table 3, all new compounds in present invention show low redox potential values. When compared with the potential value of Trx or GSH, Data confirm that new compounds are real 2-Cys-Prx mimetics capable of undergoing a peroxidase reaction coupled with Trx/TR redox system.

### Result 4: Cytotoxicity Test

Cytotoxicity tests were performed against vascular endothelial cells, vascular smooth muscle cells and hepatocytes. After treatment with ascending concentrations of each compound, the concentration of test compound corresponding to 50% cell viability (CC₅₀) was measured. Data are shown in the table below.

TABLE 5

Cell cytotoxicity (CC₅₀)

| | Smooth | Endothelial | Liver |
|---|---|---|---|
| | muscle cell | cell | cell |
| Compound | (μM) | (μM) | (μM) |
| 1 | >500 | >500 | 230.1 |
| 2 | 333.4 | >500 | >500 |
| 3 | >500 | 322.5 | 82.56 |
| 4 | 128.1 | 316.7 | 98 |
| 5 | 148.8 | 289 | >500 |
| 6 | 75.65 | 34.74 | 341.3 |
| 7 | >500 | >500 | >500 |
| 8 | 340.9 | >500 | 312.4 |
| 12 | >500 | — | >500 |
| 13 | >500 | — | >500 |
| 14 | >500 | — | — |
| 15 | 98.45 | 113.6 | 156.9 |
| 19 | >500 | >500 | >500 |
| 23 | >500 | — | 123.3 |
| A5 | 20.3 | 81.9 | 62.3 |

As a result of the measurement, except for Compound 6, most of new compounds have a higher CC₅₀ above 100 μM, indicating very low cytotoxicity. In particular, some of test compounds show no cytotoxicity in the range of concentrations used in the test (10 μM to 1 mM). This indicates that new compounds have very excellent safety margin compared to a control compound belonging to epidithiodioxopiperazine class (A5).

### Result 5: Analysis of PDGF-Mediated Signaling Pathway in Vascular Smooth Muscle Cells

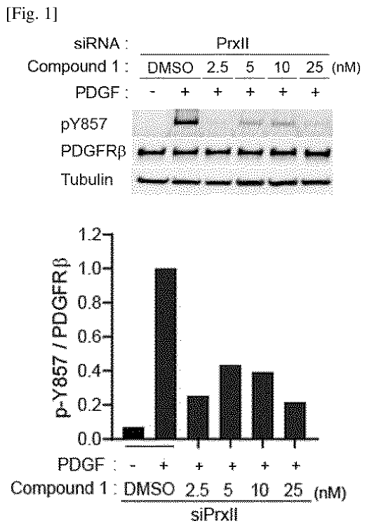

In human aortic smooth muscle cell (HASMC) and human pulmonary artery smooth muscle cell (PASMC) in which PrxII was knocked out by transfecting the PrxII siRNA, the ability of test compounds to replace the cellular function of PrxII was evaluated. That is, the efficacy of the new compounds was verified in terms of signaling pathways induced by PDGF that regulates the growth and migration of vascular smooth muscle cells in the aorta and pulmonary artery. Activation of PDGF-induced signaling pathway was analyzed by an immunoblot method using an antibody that specifically recognizes phosphorylation of Tyr 857, which is a major phosphorylation site on PDGF receptor (PDGFRβ). The results are shown in FIGS. 1 to 3.

FIG. 1 shows the effect of compound 1 on the degree of phosphorylation of Tyr 857 on PDGFRβ, which had been augmented by PrxII knock-down in HASMCs, in a concentration-dependent manner. It shows that compound 1 significantly reduces phosphorylation at concentrations above 2.5 nM.

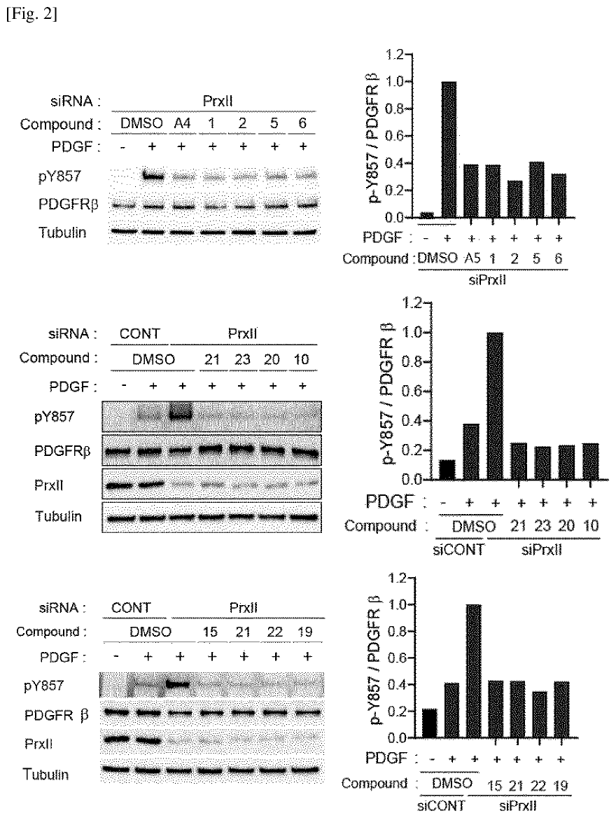

FIG. 2 shows the effect of Compound 1, 2, 5, 6, 10, 15, 19, 20, 21, 22, and 23 on the degree of phosphorylation of Tyr 857 on PDGFR, which had been augmented by PrxII knock-down in HASMCs. As shown in figure, all compounds tested strongly abolish the phosphorylation of PDGFR in HASMC depleted of PrxII expression.

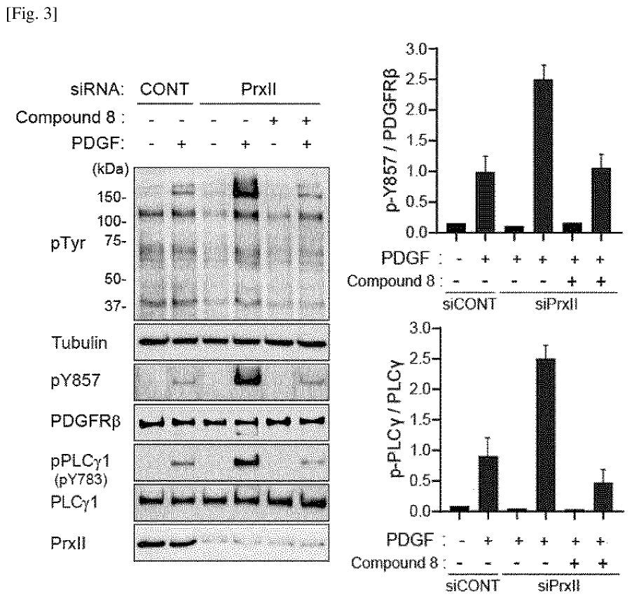

FIG. 3 shows the effect of compound 8 on the degree of total tyrosine phosphorylation and the phosphorylation of signaling proteins (e.g. PLCyl) at downstream of PDGFRβ, all of which was induced by PDGF stimulation, in PASMCs. As shown in figure, compound 8 also significantly inhibits the protein phosphorylation triggered by PDGF.

As a result of the evaluation, the ability of new compounds to regulate the PDGF signaling pathway is improved ten times better than that of control compound belonging to epidithiodioxopiperazine class (A5). New compounds in present invention also reverse abnormal PDGF signaling augmented by depletion of PrxII expression to a normal level.

### Result 6: Analysis of VEGF-Mediated Signaling Pathway in Vascular Endothelial Cells

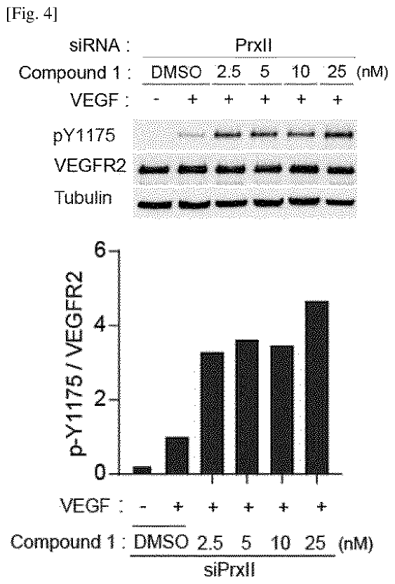

In human aortic endothelial cell (HAEC) and human pulmonary artery endothelial cell (PAEC) in which PrxII was knocked out by transfecting the PrxII siRNA, the ability of test compounds to replace the cellular function of PrxII was evaluated. That is, the efficacy of the new compounds was verified in terms of signaling pathways induced by VEGF that regulates the growth and migration of vascular endothelial cells in the aorta and pulmonary artery. Activation of VEGF-induced signaling pathway was analyzed by an immunoblot method using an antibody that specifically recognizes phosphorylation of Tyr 1175, which is a major phosphorylation site on VEGF receptor-2 (VEGFR2). The results are shown in FIGS. 4 to 6.

FIG. 4 shows the effect of Compound 1 on the degree of phosphorylation of Tyr 1175 on VEGFR2, which had been augmented by PrxII knock-down in HAECs, in a concentration-dependent manner. It shows that compound 1 significantly reduces phosphorylation at concentrations above 2.5 nM.

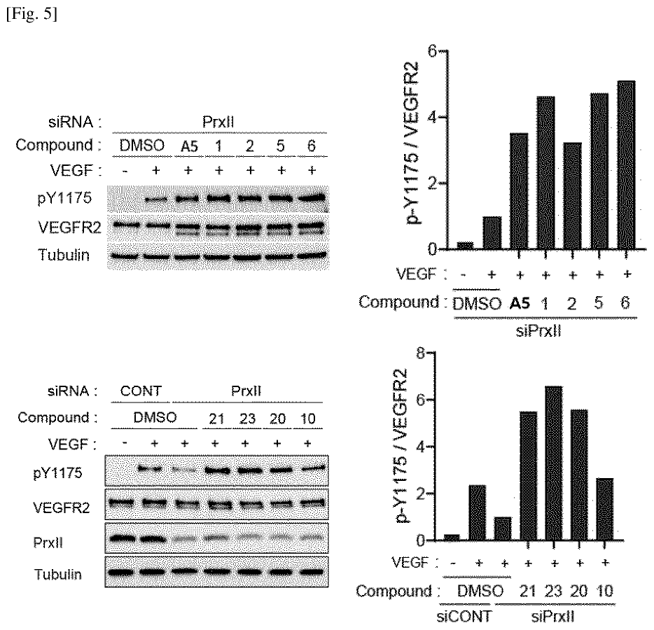

FIG. 5 shows the effect of Compound 1, 2, 5, 6, 10, 20, 21, and 23 on the degree of phosphorylation of Tyr 1175 on VEGFR2, which had been augmented by PrxII knock-down in HAECs. As shown in figure, all compounds tested strongly abolish the phosphorylation of VEGFR2 in HAECs depleted of PrxII expression.

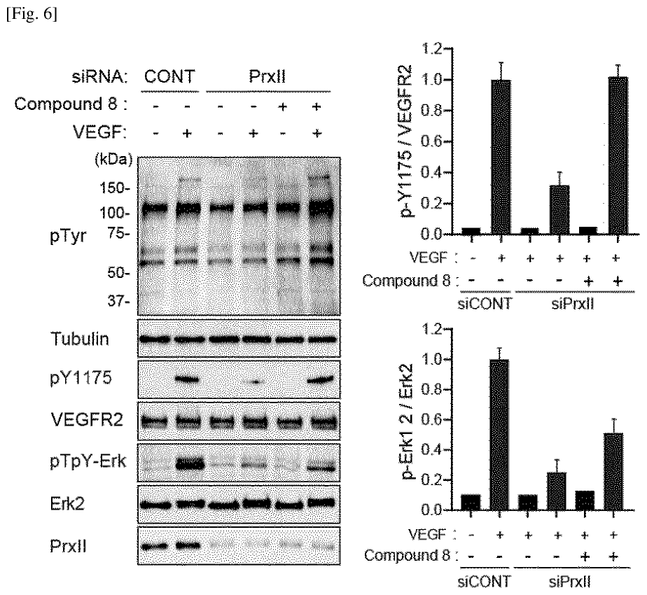

FIG. 6 shows the effect of compound 8 on the degree of total tyrosine phosphorylation and the phosphorylation of signaling proteins (e.g. ERK2) at downstream of VEGFR2, all of which was induced by VEGF stimulation, in PAECs. As shown in figure, Compound 8 also significantly inhibits the protein phosphorylation triggered by VEGF.

As a result of the evaluation, the ability of new compounds to regulate the VEGF signaling pathway is improved ten times better than that of control compound belonging to epidithiodioxopiperazine class (A5). New compounds in present invention also reverse abnormal VEGF signaling augmented by depletion of PrxII expression to a normal level.

### Result 7: Proliferation Analysis in the PDGF-Stimulated Smooth Muscle Cells

In human aortic smooth muscle cell (HASMC) and human pulmonary artery smooth muscle cell (PASMC) in which PrxII was knocked out by transfecting the PrxII siRNA, the ability of test compounds to inhibit the PDGF-induced proliferation of vascular smooth muscle cells was evaluated. The results are shown in Table 6 below. In Table 6, the degree of proliferation in the compound-treated groups is expressed as percent of the degree of proliferation in untreated control group. The concentrations of test compounds corresponding to 50% growth inhibition (IC₅₀) is also listed.

TABLE 6

| Conc. (nM) | 0 | 2.5 | 5 | 10 | 25 | 50 | 100 | IC₅₀ (nM) |
|---|---|---|---|---|---|---|---|---|
| | Degree of HASMC proliferation (%) | | | | | | | |
| Compound 1 | 100.0 | 70.0 | 43.9 | 17.1 | 1.5 | — | — | 4.18 |
| Compound 5 | 100.0 | 62.3 | 52.4 | 36.9 | 9.7 | — | — | 4.87 |
| Compound 8 | 100.0 | 75.1 | 40.1 | 46.9 | 30.2 | — | — | 6.46 |
| Compound 21 | 100.0 | 88.1 | 63.9 | 27.2 | 4.0 | — | — | 6.41 |
| Compound 23 | 100.0 | 89.8 | 54.3 | 34.4 | 20.4 | — | — | 6.85 |
| Compound 20 | 100.0 | 85.8 | 58.7 | 34.9 | 12.3 | — | — | 6.69 |
| Compound 10 | 100.0 | 75.5 | 53.8 | 31.1 | 4.6 | — | — | 5.48 |
| | Degree of PASMC proliferation (%) | | | | | | | |
| Compound 1 | 100.0 | 70.8 | 49.8 | 29.4 | 14.3 | 0.0 | 0.0 | 5.01 |
| Compound 5 | 100.0 | 71.3 | 48.3 | 37.5 | 26.8 | 1.9 | 2.5 | 5.64 |
| Compound 8 | 100.0 | 57.7 | 41.7 | 24.8 | 5.7 | 0.0 | 0.0 | 3.52 |
| Compound 21 | 100.0 | 69.9 | 49.6 | 18.7 | 6.0 | 7.1 | 0.0 | 4.51 |
| Compound 23 | 100.0 | 70.8 | 43.3 | 32.9 | 14.6 | 3.2 | 0.0 | 4.76 |
| Compound 20 | 100.0 | 82.2 | 51.5 | 31.6 | 21.1 | 7.3 | 5.2 | 6.17 |
| Compound 10 | 100.0 | 90.5 | 54.1 | 44.1 | 21.9 | 17.5 | 7.0 | 8.28 |

As shown in the table above, new compounds in the present invention inhibit the proliferation of HASMCs and PASMCs in a concentration-dependent manner, and show a growth-inhibiting activity started from concentration as low as 2.5 nM.

### Result 8: Proliferation Analysis in the VEGF-Stimulated Endothelial Cells

In human aortic endothelial cell (HAEC) and human pulmonary artery endothelial cell (PAEC) in which PrxII was knocked out by transfecting the PrxII siRNA, the ability of test compounds to augment the VEGF-induced proliferation of vascular endothelial cells was evaluated. The results are shown in Table 7 below. In Table 7, the degree of proliferation in the compound-treated groups is expressed as percent of the degree of proliferation in untreated control group. The effective concentrations of test compounds increasing cell proliferation by 50% (EC₅₀) is also listed.

TABLE 7

| Conc. (nM) | 0 | 2.5 | 5 | 10 | 25 | 50 | 100 | EC₅₀ (nM) |
|---|---|---|---|---|---|---|---|---|
| | Degree of HAEC proliferation (%) | | | | | | | |
| Compound 1 | 100.0 | 113.8 | 143.7 | 162.9 | 187.8 | 164.7 | 170.2 | 4.93 |
| Compound 5 | 100.0 | 112.2 | 129.7 | 165.6 | 191.6 | 172.7 | 180.1 | 5.31 |
| Compound 8 | 100.0 | 107.3 | 147.3 | 186.3 | 204.8 | 157.0 | 161.6 | 5.04 |
| Compound 21 | 100.0 | 105.0 | 122.7 | 154.6 | 190.1 | 202.0 | 141.2 | 9.96 |
| Compound 23 | 100.0 | 109.4 | 122.6 | 161.3 | 187.6 | 177.1 | 133.0 | 9.72 |
| Compound 20 | 100.0 | 116.4 | 120.3 | 136.3 | 155.9 | 169.5 | 187.6 | 10.16 |
| Compound 10 | 100.0 | 111.6 | 122.8 | 127.6 | 140.7 | 175.1 | 159.0 | 19.32 |
| | Degree of PAEC proliferation (%) | | | | | | | |
| Compound 1 | 100.0 | 104.7 | 120.0 | 143.8 | 164.3 | 158.8 | 145.5 | 9.54 |
| Compound 5 | 100.0 | 106.1 | 129.7 | 152.3 | 165.6 | 167.7 | 147.4 | 5.03 |
| Compound 8 | 100.0 | 108.5 | 124.9 | 143.4 | 164.8 | 158.1 | 144.0 | 5.12 |
| Compound 21 | — | — | — | — | — | — | — | — |
| Compound 23 | 100.0 | 109.2 | 122.9 | 148.3 | 173.3 | 155.3 | 133.9 | 5.20 |
| Compound 20 | 100.0 | 109.2 | 124.3 | 133.5 | 166.5 | 180.9 | 180.4 | 10.28 |
| Compound 10 | — | — | — | — | — | — | — | — |

As shown in the table above, new compounds in the present invention promote the proliferation of HAECs and PAECs in a concentration-dependent manner, and show a growth-promoting activity started from concentration as low as 2.5 nM.

### Result 9: Preclinical Animal Study for Pulmonary Arterial Hypertension

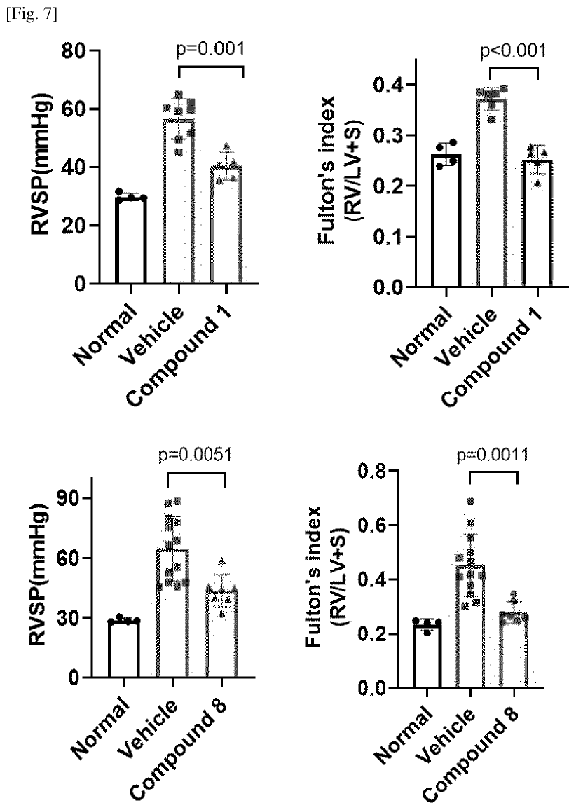

The therapeutic effect of new compounds on pulmonary arterial hypertension was evaluated in a preclinical SuHx rat model. For establishing PAH model, rats were administered with Sugen (VEGFR2 inhibitor) and placed in a normobaric hypoxic condition (10% O₂) for 3 weeks. Rats were moved to normoxic condition and orally administered with control vehicle or compound 1 or 8 (P.O., once daily, 0.1 mg/kg) for 5 weeks. Thereafter, right ventricular systolic pressure (RVSP) and right ventricular hypertrophy (Fulton's index) were measured at the end of treatment. The results are shown in FIGS. 7-9.

FIG. 7 shows that Compound 1 and 8 significantly reduces RVSP and RV hypertrophy compared to vehicle control group.

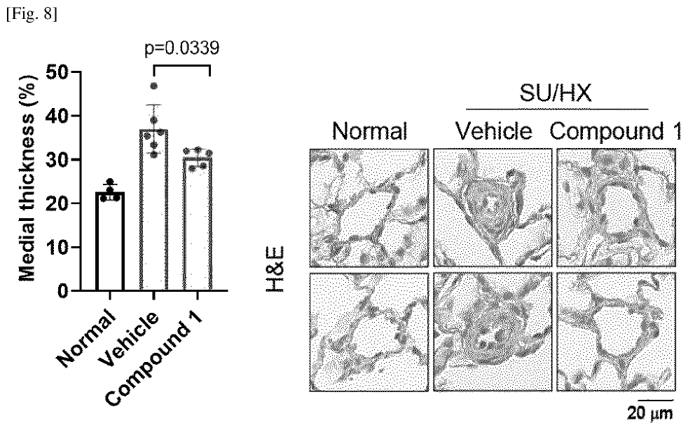

FIG. 8 shows that Compound 1 widens the pulmonary arterial vessels which remains to be severely occluded in the vehicle control group. Data indicate that treatment of Ccompound 1 induces normal blood flow in the lumen of the blood vessel, which results in the reduction of RVSP and RV hypertrophy.

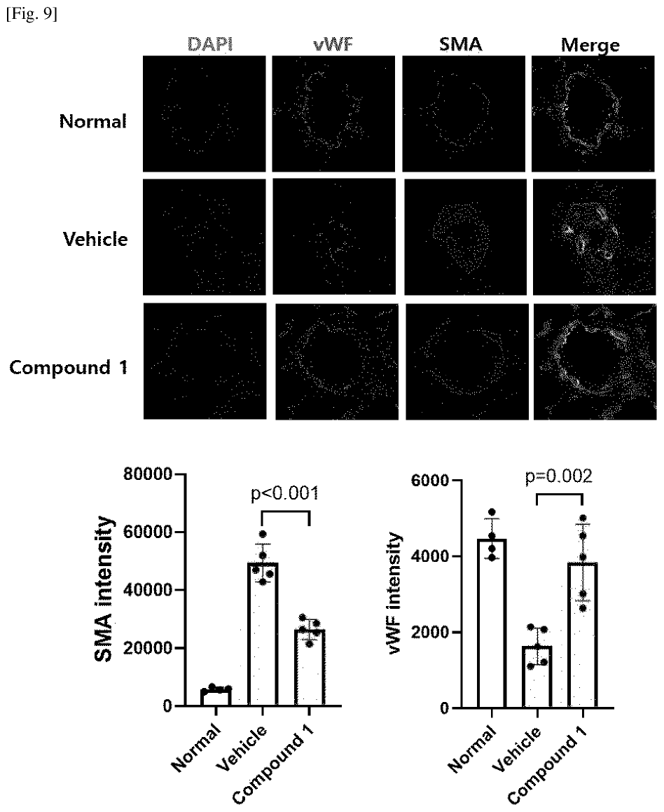

FIG. 9 shows an immunostaining images of endothelium and medial SMC layer in pulmonary arterial vessels. Specifically, the lung tissue sections were immunostained with an endothelial cell-specific vWF antibody and a smooth muscle cell-specific SMA antibody. The results demonstrate that the endothelial damage and medial thickness due to SMC hyperplasia occur in vehicle control group. However, treatment of Compound 1 induces the recovery of endothelial layer (vWF-labeled ECs) as well as the reduction of medial thickness (SMA-labeled SMCs).

1. A compound of Chemical Formula 1 or Chemical Formula 2: 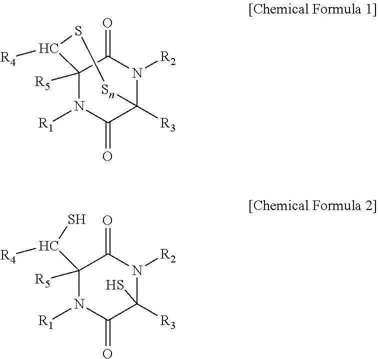
or a pharmaceutically acceptable salt thereof,
in the Chemical Formula 1 and 2,
n is an integer of from 1 to 3, R₁ and R₂ are each independently C₁₋₃alkyl, C₁₋₃alkoxy-C₁₋₃alkyl, —(CH₂)₁₋₃—C(R′)(R″)OH, —(CH₂)₁₋₃—N(R′)(R″), —(CH₂)₀₋₃-alkenyl, —(CH₂)₀₋₃-alkynyl, —(CH₂)₀₋₃—C(R′)(R″)CO₂H, —(CH₂)₀₋₅-heterocycloalkyl, —(CH₂)₀₋₅-cycloalkyl, —(CH₂)₀₋₅-aryl, or —(CH₂)₀₋₅-heteroaryl, wherein the alkyl, heterocycloalkyl, cycloalkyl, aryl and heteroaryl are unsubstituted or optionally substituted with one or more substituents selected from the group consisting of C₁₋₃alkyl, —CF₃, C₁₋₃alkoxy, —OCF₃, halogen, CN, amino, —N(R′)(R″), —OH, —COOH, —COO—C₁₋₃alkyl, and =O, wherein R′ and R″ are each independently H or C₁₋₃alkyl,
R₃ is C₁₋₃alkyl, —(CH₂)O₃-aryl, or —(CH₂)₀₋₃-heteroaryl, wherein the aryl or heteroaryl is unsubstituted or optionally substituted with one or more substituents selected from the group consisting of C₁₋₃alkyl, —CF₃, C₁₋₃ alkoxy, —OCF₃, halogen, —CN, amino, —OH, and —COOH; or
R₂ and R₃ are linked together and fused with piperazinedione present in Chemical Formula 1 to form one of the following structures: 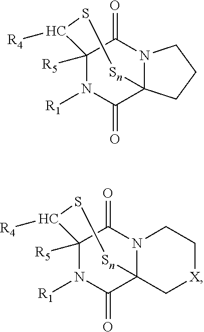
wherein,
X is S, SO₂, CH₂, O or NR₆, wherein R₆ is H or C₁₋₃alkyl,
R₄ is H or C₁₋₃alkyl, and
R₅ is H, C₁₋₃alkyl, —(CH₂)₁₋₂-aryl, or —(CH₂)₁₋₂-heteroaryl.

2. The compound of claim 1 or a pharmaceutically acceptable salt thereof,
n is an integer of from 1 to 3,
R₁ and R₂ are each independently C₁₋₃alkyl, —(CH₂)₁₋₂-heterocycloalkyl, —(CH₂)₁₋₂-aryl, or —(CH₂)₁₋₂-heteroaryl, wherein the alkyl, heterocycloalkyl, aryl, and heteroaryl are unsubstituted or optionally substituted with one or more substituents selected from the group consisting of C₁₋₃ alkyl, —CF₃, C₁₋₃alkoxy, CN, halogen, —OH, —COOH, and —COO—C₁₋₃ alkyl,
R₃ is C₁₋₃alkyl, —CH₂-aryl, or —CH₂-heteroaryl, wherein the aryl or heteroaryl is unsubstituted or optionally substituted with one or more substituents selected from the group consisting of methyl, methoxy, halogen, —CN, amino, —OH, and —COOH; or
R₂ and R₃ are linked together and fused with piperazinedione present in Chemical Formula 1 to form one of the following structures: 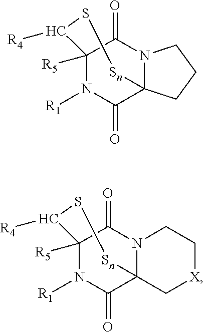
wherein,
X is S, SO₂, CH₂, O or NR₆, wherein R₆ is H or C₁₋₃alkyl,
R₄ is H or C₁₋₃alkyl, and
R₅ is H, C₁₋₃alkyl, or —(CH₂)₁₋₂-aryl.

3. The compound of claim 2 or a pharmaceutically acceptable salt thereof,
n is 1,
R₁ and R₂ are each independently C₁₋₃alkyl, —CH₂-piperidyl, —CH₂— morpholinyl, —CH₂-piperazinyl, —CH₂-phenyl, —CH₂-naphthyl, —CH₂— pyridyl, —CH₂-quinolinyl, —CH₂-pyrazolyl, —CH₂-thiophen-2-yl, —CH₂-benzo[d]thiazol-2-yl, —CH₂-pyrimidyl, —CH₂-1H-imidazol-4-yl, —CH₂-1H-imidazol-2-yl, —CH₂-thiazol-4-yl, —CH₂-thiazol-5-yl, —CH₂— isoxazolyl, —CH₂-indol-2-yl, —CH₂-indol-3-yl, —CH₂-benzimidazol-5-yl, —CH₂-quinolin-4-yl, —CH₂-quinazol-2-yl, or —CH₂-quinazol-4-yl, wherein the the piperidyl, morpholinyl, piperazinyl, phenyl, naphthyl, pyridyl, quinolinyl, pyrazolyl, thiophene, benzo[d]thiazole, pyrimidyl, imidazole, thiazole, isoxazolyl, indole, benzimidazole, quinoline and quinazole are unsubstituted or optionally substituted with one or more substituents selected from the group consisting of C₁₋₃alkyl, —CF₃, C₁₋₃ alkoxy, CN, halogen, and —COO—C₁₋₃alkyl,
R₃ is C₁₋₃alkyl or —CH₂-aryl, or
R₂ and R₃ are linked together and fused with piperazinedione present in Chemical Formula 1 to form one of the following structures: 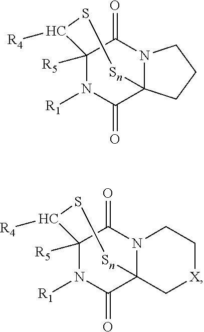
wherein,
X is O or NR₆, wherein R₆ is methyl,
R₄ is H, and
R₅ is H.

4. The compound of claim 1 or a pharmaceutically acceptable salt thereof, wherein the compound is
1,6,8-trimethyl-2,3-dithia-6,8-diazabicyclo[3.2.2]nonan-7,9-dione (Compound 1),
6-benzyl-1,8-dimethyl-2,3-dithia-6,8-diazabicyclo[3.2.2]nonan-7,9-dio ne (Compound 2),
1,8-dimethyl-6-(3,4,5-trimethoxybenzyl)-2,3-dithia-6,8-diazabicyclo[3.2.2]nonan-7,9-dione (Compound 3),
6-(3,5-difluorobenzyl)-1,8-dimethyl-2,3-dithia-6,8-diazabicyclo[3.2.2]n onan-7,9-dione (Compound 4),
1,8-dimethyl-6-(quinolin-2-ylmethyl)-2,3-dithia-6,8-diazabicyclo[3.2.2]nonan-7,9-dione (Compound 5),
1,8-dimethyl-6-(pyridin-2-ylmethyl)-2,3-dithia-6,8-diazabicyclo[3.2.2]nonan-7,9-dione (Compound 6),
11-benzyltetrahydro-5H,7H-4,9a-(epiminomethano)pyrrolo[2,1-c][1,2,4]dithiazepin-5,10-dione (Compound 7),
12-benzyltetrahydro-5H,10H-4,10a-(epiminomethano)[1,4]oxazino[3,4-c][1,2,4]dithiazepin-5,11-dione (Compound 8),
12-(pyridin-4-ylmethyl)tetrahydro-5H,10H-4,10a-(epiminomethano)[1,4]oxazino[3,4-c][1,2,4]dithiazepin-5,11-dione (Compound 9),
12-ethyltetrahydro-5H,10H-4,10a-(epiminomethano)[1,4]oxazino[3,4-c][1,2,4]dithiazepin-5,11-dione (Compound 10),
11-(pyridin-4-ylmethyl)tetrahydro-5H,7H-4,9a-(epiminomethano)pyrro lo[2,1-c][1,2,4]dithiazepin-5,10-dione (Compound 11),
1,8-dimethyl-6-((6-methylpyridin-2-yl)methyl)-2,3-dithia-6,8-diazabicy clo[3.2.2]nonan-7,9-dione (Compound 12),
1,8-dimethyl-6-((1-methyl-1H-pyrazol-4-yl)methyl)-2,3-dithia-6,8-diaz abicyclo[3.2.2]nonan-7,9-dione (Compound 13),
1,8-dimethyl-6-(thiophen-2-ylmethyl)-2,3-dithia-6,8-diazabicyclo[3.2.2]nonan-7,9-dione (Compound 14),
6-(benzo[d]thiazol-2-ylmethyl)-1,8-dimethyl-2,3-dithia-6,8-diazabicycl o[3.2.2]nonan-7,9-dione (Compound 15),
1,6-dimethyl-8-(pyrimidin-2-ylmethyl)-2,3-dithia-6,8-diazabicyclo[3.2.2]nonan-7,9-dione (Compound 16),
1,6-dimethyl-8-((1-methyl-1H-imidazol-4-yl)methyl)-2,3-dithia-6,8-dia zabicyclo[3.2.2]nonan-7,9-dione (Compound 17), methyl
4-((1,6-dimethyl-7,9-dioxo-2,3-dithia-6,8-diazabicyclo[3.2.2]nonan-8-y l)methyl)benzoate (Compound 18),
1-benzyl-6,8-dimethyl-2,3-dithia-6,8-diazabicyclo[3.2.2]nonan-7,9-dio ne (Compound 19),
6,8-diethyl-1-methyl-2,3-dithia-6,8-diazabicyclo[3.2.2]nonan-7,9-dione (Compound 20),
12-benzyl-9-methylhexahydro-5H-4,10a-(epiminomethano)pyrazino[2, 1-c][1,2,4]dithiazepin-5,11-dione (Compound 21),
12-benzyl-9-methylhexahydro-5H-4,10a-(epiminomethano)pyrazino[2, 1-c][1,2,4]dithiazepin-5,11-dione (Compound 22),
12-benzyl-9-methylhexahydro-5H-4,10a-(epiminomethano)pyrazino[2, 1-c][1,2,4]dithiazepin-5,11-dione (Compound 23),
6-benzyl-1,4,8-trimethyl-2,3-dithia-6,8-diazabicyclo[3.2.2]nonan-7,9-di one (Compound 24), or
6-benzyl-1,4,8-trimethyl-2,3-dithia-6,8-diazabicyclo[3.2.2]nonan-7,9-di one (Compound 25).

5. A method of treating or preventing a vascular disease, comprising administering a therapeutically effective amount of a compound of any one of claims 1 to 4 or a pharmaceutically acceptable salt thereof to a subject in need of prevention or treatment of the vascular disease or a subject suspected of the vascular disease.

6. The method claim 5, wherein the vascular disease is any one selected from the group consisting of hypertension, ischemic coronary artery disease, cerebral artery occlusion, arteriosclerosis, peripheral arterial occlusive disease, thromboembolism, diabetic foot lesion, venous ulcer, deep vein thrombosis, vasospasm, arteritis and vascular restenosis.

7. The method of claim 6, wherein the vascular disease is ischemic coronary artery disease, arteriosclerosis, vascular restenosis or pulmonary arterial hypertension.

8. Use of a compound of any one of claims 1 to 4 or a pharmaceutically acceptable salt thereof for the manufacture of a medicament for the treatment or prevention of a vascular disease.

9. The use of claim 8, wherein the vascular disease is any one selected from the group consisting of hypertension, ischemic coronary artery disease, cerebral artery occlusion, arteriosclerosis, peripheral arterial occlusive disease, thromboembolism, diabetic foot lesion, venous ulcer, deep vein thrombosis, vasospasm, arteritis and vascular restenosis.

10. The use of claim 9, wherein the vascular disease is ischemic coronary artery disease, arteriosclerosis, vascular restenosis or pulmonary arterial hypertension.

11. A method for preparing the compound represented by Chemical Formula 3, comprising reacting a
6-(1-hydroxyalkyl)piperazin-2,5-dione derivative represented by Chemical Formula 4 with (a) sulfur (Ss) and (b) lithium bis(trimethylsilyl)amide (LiHMDS) or sodium bis(trimethylsilyl)amide (NaHMDS). 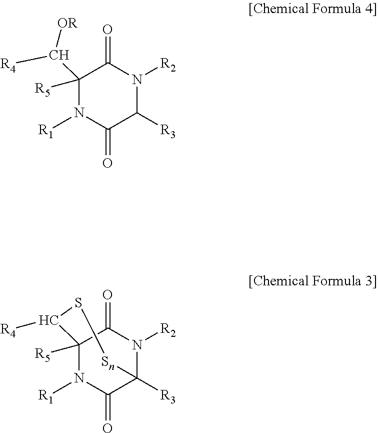
In the Chemical Formula 4, R₁, R₂, R₃, and R₄ are the same as in Chemical Formula 1 of claim 1; R₅ is H; and R is a protecting group,
In the Chemical Formula 3, R₁, R₂, R₃, and R₄ are the same as in Chemical Formula 1 of claim 1; R₅ is H; and n is 2 or 3.

12. A method for preparing a compound represented by the following Chemical Formula 1′, comprising
(S1) reacting a compound represented by Chemical Formula 4 with (a) sulfur (S₈) and (b) LiHMDS (lithium bis(trimethylsilyl)amide) or NaHMDS (sodium bis(trimethylsilyl)amide) to obtain a compound represented by Chemical Formula 3;
(S2) reducing the compound of Chemical Formula 3 to obtain a compound represented by Chemical Formula 2; and
(S3) forming an intramolecular disulfide crosslink from the compound represented by Chemical Formula 2. 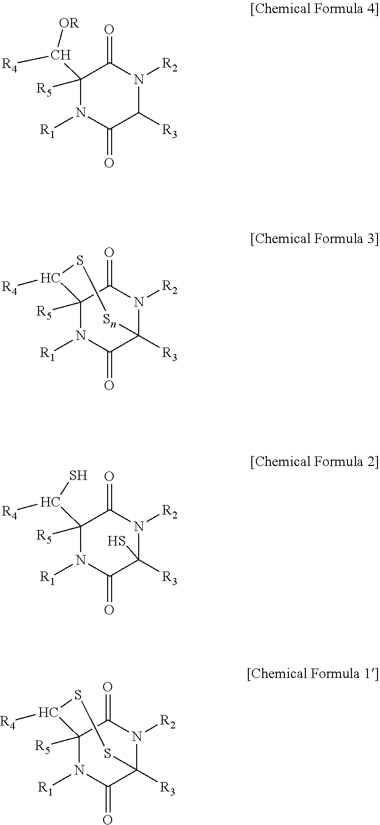
In the Chemical Formula 4, 3, 2 and 1′, R₁, R₂, R₃, and R₄ are the same as in Chemical Formula 1 of claim 1; R₅ is H; R is a protecting group; and n is 2 or 3.
